(19) United States
(12) Patent Application Publication
(10) Pub. No.: US 2022/0273622 A1
(43) Pub. Date: Sep. 1, 2022

(54) PROPHYLAXIS AND REVERSAL OF STIMULANT AND OPIOID/OPIATE OVERDOSE AND/OR TOXIC EXPOSURE

(71) Applicant: Torralva Medical Therapeutics LLC, Portland, OR (US)

(72) Inventor: Phillip R. Torralva, Portland, OR (US)

(73) Assignee: Torralva Medical Therapeutics LLC, Portland, OR (US)

(21) Appl. No.: 17/749,082

(22) Filed: May 19, 2022

Related U.S. Application Data
(63) Continuation of application No. PCT/US2020/061611, filed on Nov. 20, 2020.
(60) Provisional application No. 62/938,466, filed on Nov. 21, 2019.

(51) Int. Cl. A61K 31/4168; A61K 31/485; A61K 31/216; A61K 31/517; A61K 31/18; A61K 31/221; A61K 31/5517; A61K 31/4166; A61K 31/4045; A61K 31/165; A61K 31/138; A61K 31/58; A61K 9/00; A61P 25/36

(52) U.S. Cl. CPC A61K 31/4168; A61K 31/485; A61K 31/216; A61K 31/517; A61K 31/18; A61K 31/221; A61K 31/5517; A61K 31/4166; A61K 31/4045; A61K 31/165; A61K 31/138; A61K 31/58; A61K 9/0073; A61P 25/36

(57) ABSTRACT

Methods are provided of preventing or reversing negative effects in a subject, which effects arise from intentional or accidental opioid or opiate exposure coupled with intentional or accidental stimulant exposure, or interactive effects of these classes of drugs (e.g. Stimulant and Synthetic Opioid Induced Vascular Events (SSOIVE) from concurrent opioid and stimulant overdose). The methods involve administering to the subject a pharmaceutical composition including therapeutically effective amounts of an α1 adrenergic receptor antagonist, together with one or more of a mu (or opioid receptor subtype) antagonist or agonist, an anticholinergic agent and/or cholinergic agents, a combined alpha-1 adrenergic antagonist and anticholinergic, a paralytic or muscle relaxant, a GABA complex antagonist, an anti-seizure/membrane stabilizer agent, an β2 adrenergic receptor agonist and/or a beta blocker; and a pharmaceutically acceptable carrier.

### CROSS-REFERENCE TO RELATED APPLICATIONS

This application is a continuation of co-pending PCT/US2020/061611, filed Nov. 20, 2020, which claims priority to and the benefit of the earlier filing date of U.S. Provisional Application No. 62/938,466, filed on Nov. 21, 2019. These earlier-filed applications are incorporated by reference herein in their entirety.

### FIELD OF THE DISCLOSURE

This disclosure relates to compositions and methods to treat (e.g., reverse and/or prevent) opiate and opioid effects in a subject. It further relates to preventing or reversing opioid/opiate overdose in the context of polysubstance use with drugs such as stimulants (e.g. methamphetamine, cocaine, etc.).

### BACKGROUND OF THE DISCLOSURE

In October of 2017, the opioid abuse epidemic was declared a national “public health emergency” in the United States of America. This declaration was based on the findings of a study by the Opioid and Drug Abuse Commission (ODAC), including that opioid-related deaths had risen from 4,000 in 1999 to over 64,000 in 2016. Opioid overdose the leading cause of death for Americans under the age of 50 (Rudd et al., Morb Mortal Wkly Rep 65:1445-1452, 2016). The Commission's report included a finding that the opioid epidemic had cost the U.S. an estimated $504 billion in 2015 alone, and is expected to cost over $1 trillion for 2018-2020.

The Center for Disease Control (CDC) reported that the highly potent synthetic opioid Fentanyl (Sublimaze™) and its analogues were the cause of death in >50% of U.S. deaths related to opioids in 2016 and estimated to be >70% for 2017 and 2018 (O'Donell & Halpin, Synthetic Fentanyl deaths rise in American opioid epidemic. U.S. CDC, Oct. 27, 2017).

Numerous public health and first responder reports indicate the failure of high dose naloxone to resuscitate overdose from illicit F/FA use, making F/FAs the number one cause of death in U.S. adults ages 18-50. With public health data increasingly indicating that naloxone is ineffective at decreasing F/FA-induced rapid fatality in the current U.S. opioid crisis, the development of reversal and prophylaxis drugs. Currently there are no Food and Drug Administration (FDA)-approved pharmacotherapies specifically for rescue treatment of F/FA-induced WCS or that can treat the unique side effect profile of F/FAs.

Like all narcotic opiates when given in sufficient quantity, fentanyl can induce significant, dose-dependent respiratory depression (RD) and apnea. Left untreated or treated inadequately, opioid-induced respiratory depression leads to hypoxia and death.

F/FAs are unique in that they can also rapidly induce severe muscle rigidity in the chest wall, diaphragm (Fentanyl or F/FA induced respiratory muscle rigidity—FIRMR), and spasm of the larynx (laryngospasm) resulting in vocal cord closure well within the therapeutic ranges used for analgesia (Grell et al., Anesth Analg 49(4):523-532, 1970; Streisand et al., Anesthesiology 78(4):629-634, 1993; Bennet et al., Anesthesiology 8(5):1070-1074, 1997; Coruh et al., Chest. 143(4):1145-1146, 2013; Ackerman et al., Anesth Prog 37(1):46-48, 1990; McClain et al., Clin Pharmacol Ther. 28:106-114, 1980). This combination of FIRMR and laryngospasm are also clinically known as “wooden chest syndrome” (WCS) or more specifically, Fentanyl or F/FA induced respiratory effects—FIRE syndrome (e.g. respiratory muscle effects and laryngospasm), which usually occurs within 1-2 minutes after rapid injection and lasts ˜8-15 minutes. Rapidity of injection is the key determinant of the severity and duration of the FIRE syndrome (Grell et al., supra). The resulting rigidity reduces chest wall compliance and makes rescue-assisted ventilation extremely difficult outside of a critical care setting or operating room. Intervention for FIRE syndrome must be immediate and aggressive to avoid death and usually includes treatment with a muscle paralytic and endotracheal intubation to secure the airway. This has been the method of choice since the underlying mechanism in humans remains unknown outside of this disclosure.

The need to combat opiate and opioid overdose is urgent, immediate, and rapidly increasing. There are currently no molecules or compounds that have been designed or exist for this specific purpose.

Similar to the significant rise in synthetic opioids reviewed above, synthetic stimulants (e.g. amphetamines, methamphetamine) and plant alkaloids (e.g. cocaine) have shown a significant increase in overdose deaths from 2010 to the present, with deaths from methamphetamine overdose increasing from 1,400 in 2010 to well over 10,000 in 2017 and similar increases for cocaine at 14,000 in 2017 compared with 3000 in 2010. Of particular significance is the fact that these numbers mirror the rise in overdoses from fentanyl, at 29,000 in 2017 versus 1000 in 2010 in the same time frame. Toxicology reports confirm that ˜70% of the stimulant overdoses show positive for fentanyl, carfentanil or other potent fentanyl analogues (Vestal, As the Opioid Crisis Peaks, Meth and Cocaine Deaths Explode, Stateline, Pew Charitable Trust, May 13, 2019; available online at pewtrusts.org/en/research-and-analysis/blogs/stateline). Additionally, current public health data also supports that the combination of stimulants and F/FAs appears to significantly increase lethality. For example, cocaine, which shares similar sympathetic effects and molecular mechanisms as methamphetamines, when mixed with fentanyl, seems to have more potent lethal effects than cocaine alone. Federal data (available online at drugabuse.gov/related-topics/trends-statistics/overdose-death-rates) shows that fentanyl and its analogs have increasingly appeared in cocaine overdose deaths. The rise in deaths involving both cocaine and fentanyl is startling and has significant implications for ongoing drug overdose crisis in the U.S. Deaths involving both cocaine and opioids have more than tripled since 2010, while cocaine deaths not involving opioids have only increased by 1.5-fold in the same time span (Cheng et al., ACS Chem Neurosci. 10(8):3486-3499, 2019). Drug overdose deaths involving cocaine rose from 3,822 in 1999 to 13,942 in 2017. As illustrated for instance in a NIDA report (NIDA. (2019, Jan. 29). Overdose Death Rates. Retrieved from the World Wide Web at drugabuse.gov/related-topics/trends-statistics/overdose-death-rates), the number of deaths in combination with any opioid has been increasing steadily since 2014 and is mainly driven by deaths involving cocaine in combination with other synthetic narcotics.

The problem, simply stated, is that the synthetic opioid fentanyl and its analogues appear to significantly augment the lethality of stimulants and are under-recognized contaminants for which there are currently no molecules or compounds that exist or have been designed for reduction of death associated with their combination. There are no reversal or prophylaxis drugs or compounds for Stimulant and Synthetic Opioid Induced Vascular Events (SSOIVE) for resuscitation from or prevention of opioid and stimulant overdose. Considering that death rates from stimulants contaminated with fentanyl/fentanyl analogues or other opioids represent ˜70% of lethal overdoses from stimulants, there is an urgent need to develop drugs or compounds that can increase survival rates and decrease the risk of death associated with the combinations of these drugs. Combining fentanyl/ fentanyl analogues with stimulants was unknown prior to the current opioid crisis; prior to the subject disclosure, no treatments with prophylaxis or reversal agents had been described for this combination of drugs.

### SUMMARY OF THE DISCLOSURE

Disclosed herein is the underlying mechanism of increased risk from the interaction of stimulants and the synthetic opioid fentanyl (e.g. fentanyl analogues), also referred to as Stimulant and Synthetic Opioid Induced Vascular Events (SSOIVE). Also disclosed are molecular targets identified based on the discoveries described herein.

This disclosure examines and describes mechanisms developed through the inventor's clinical observation and experience with FIRE syndrome in the fields of anesthesiology and addiction medicine, and the inventor's clinical observations demonstrating that FIRE syndrome is the key cause of rapid death and escalating numbers of death in the current F/FA driven opioid crisis. This disclosure also examines and describes the underlying mechanisms developed through the inventor's clinical practice involving individuals who suffer from stimulant and polysubstance use disorder, in which increased mortality is seen when the synthetic opioid fentanyl (or a comparable analogue) is used intentionally or unintentionally with stimulants (e.g., methamphetamine, cocaine). The lethal effects of either drug is augmented by modulation of norepinephrine levels by each drug and directly relate to the underlying pharmacologic mechanisms whereby each drug has lethal effects on vascular and respiratory systems.

Provided herein is a description of the systematic development of a new generation of opioid and stimulant reversal drugs and treatments designed to simultaneously and effectively antagonize both mu opioid receptor and other opioid receptor subtypes (kappa and delta) and the receptor(s) involved with fentanyl induced muscle rigidity (FIMR), fentanyl induced respiratory muscle rigidity (FIRMR), vocal cord closure (laryngospasm), and/or FIRE syndrome (FIRE syndrome=FIRMR+laryngospasm) and the vascular and hemodynamic effects of stimulants through these same alpha adrenergic receptors. The inventor has demonstrated that fentanyl and potent analogues of fentanyl such as carfentanil can increase or facilitate noradrenergic activity. It has surprisingly been discovered (based on animal models) and human alpha 1 adrenoreceptor binding assays performed by the inventor that in addition to mu opioid receptors, F/FAs binding of alpha adrenergic and cholinergic receptors (e.g. muscarinic and nicotinic) contributes to and may be the most significant underlying cause of FIRE syndrome. The inventor has further demonstrated in an animal model that vocal cord closure and chest wall rigidity occur simultaneously after high dose fentanyl (100 mcg/kg) within 15-30 seconds after intravenous bolus, persist for -90 seconds, whereupon the heart becomes asystolic and arterial pressure falls to 0 (zero) mm Hg and the animal cannot be resuscitated without the administration of therapeutic agents. All respiratory effort ceases at the time onset of vocal cord closure (e.g. 15-30 seconds after IV bolus). This effect is specific to F/FA and is not demonstrated with morphine. Based in part on these discoveries, this disclosure provides a clear methodology for the development of effective treatment compounds for prophylaxis and reversal of overdose and toxicity from F/FAs.

Conventional opiate reversal technology (e.g. naloxone, naltrexone) exclusively targets the mu-opioid receptor, and to a lesser extent the opioid receptor subtypes (kappa and delta), and uses these mu-opioid receptor antagonists for pharmacologic reversal of opioid-induced respiratory depression and over-sedation from both morphine alkaloid derived and synthetic opioids (e.g. F/FAs, meperidine, methadone). As described herein, respiratory depression can occur with all opioids, but FIRE syndrome appears to be a unique and lethal side effect of F/FAs that is clinically and neuropharmacologically distinct from morphine derived alkaloids and the effects of opioids at opioid receptors. It becomes more readily understood by the teachings herein that, due to the unique side effect profile of fentanyl and fentanyl analogs (F/FAs), conventional single therapy with naloxone is no longer adequate, safe or cost effective for treatment of overdose or toxic exposure related to F/FAs or “fentanyl-tainted heroin”.

The use of either pure fentanyl, fentanyl analogs (e.g. synthetic opioids) or the concurrent use of F/FAs with heroin or other morphine derived opiates (e.g. natural alkaloids), creates a unique problem in the conventional treatment of narcotic overdose (e.g. opiates and opioids). This problem is further compounded by the overlapping of noradrenergic mechanisms that cause the effects and side effects of stimulants and the fentanyl class of molecules (e.g. cocaine and/or methamphetamine increase norepinephrine release and block reuptake. Solutions to this problem are by embodiments of the current disclosure. As demonstrated herein, F/FAs are mechanistically unique from morphine, particularly in their effects on the upper airway (larynx and vocal cords) and in FIRE syndrome. This disclosure describes receptor populations that drive the clinical effects of FIRE syndrome. These receptor populations in turn suggest a multi-site effect that requires multiple drugs in combination as a compound for optimal treatment (e.g., combinations of drugs that specific target mu opioid receptors, alpha-1 adrenergic receptors, muscarinic cholinergic receptors and beta blockers). This disclosure teaches how to make these combination compounds and how to administer them for treatment and prevention (e.g. the conditions of administration). Additionally, this disclosure teaches that the increased lethality seen with synthetic opioid(s) combined with stimulant(s) results from the overlapping mechanisms of each of these drug classes that subsequently increases noradrenergic driven physiologic effects that ultimately cause catastrophic injury to cardiac, vascular, and respiratory systems. Additionally, this disclosure teaches that the increased lethality seen with synthetic opioids combined with stimulants results from the overlapping mechanisms of each of these drug classes that subsequently increase noradrenergic driven physiologic effects that ultimately cause catastrophic injury to cardiac, vascular and respiratory systems.

Prior to the teachings herein, there was no focus on developing therapeutic agents specifically for the reversal or prophylaxis treatment of F/FA induced WCS. This is due at least in part to a pervasive misunderstanding in the medical and research community of the basic mechanism of action (MOA) involved in F/FAs overdose, and a consistent misperception among health care professionals and researchers in addictionology that F/FAs are simply more potent versions of morphine and heroin. The misconception is that mu opioid receptor-mediated respiratory depression is the main cause of death after F/FA overdose, and therefore the administration of conventional medications (mu opioid receptor antagonists, such as naloxone) in larger amounts would seem to be the logical solution or treatment to compensate for the increased potency of F/FAs (Baumann et al., Trends Pharmacol Sci. 39(12):995-998, 2018). Similarly, the risk of negative complications due to high dose naloxone (e.g. pulmonary edema, cardiac arrhythmias) are not commonly known among these same practitioners. These two factors have contributed significantly to the ongoing morbidity and mortality from F/FAs related overdose, have limited the development of new drugs or compounds specifically for this purpose and the understanding of underlying mechanisms of F/FAs in FIRE syndrome. The advances described herein address these problems directly.

This disclosure describes methodologies for treating opioid overdose and F/FAs related overdose by using a “multi-systems treatment approach” through the use of compounds/ combinations of molecules that concurrently target multiple physiologic systems and symptoms to optimize opioid overdose reversal involving F/FAs and combinations of F/FAs with heroin and other morphine derived alkaloids.

There is provided herein a platform of compounds that are all part of a single invention and singular outcome (overdose survival) that is adapted to variations in human physiology and adaptable to variations of opioid molecules overlapping in their mechanisms of overdose and death. These compounds share the same underlying mechanism and function of concurrently blocking or reversing the effects of natural opiate alkaloids, and/or the effects of synthetic opiate receptor agonists on opiate receptors and other receptor types, in the body and brain of mammalian system that contribute to the lethal effects of opiate and opioid overdose. For the purposes of this disclosure, opioid overdose with F/FAs includes FIRE syndrome in addition to respiratory depression, and optimal treatment involves the concurrent treatment of both clinical presentations and their underlying mechanisms. The technology described here provides a series of compounds and composition using established recognized therapeutic compounds (drugs) and other molecules that selectively bind receptors and receptor subtypes in brain and body regions responsible for FIMR and F/FAs overdose-related physical sequelae (such as FIRE syndrome and SSOIVE).

In specific embodiments, this disclosure offers a multimodal approach to concurrently affect central and peripheral effect sites of opiates and opioids, and favorably impact the physical symptoms of overdose such as vascular compromise; lowered hemodynamics, blood pressure, heart rate; increased vagal tone; chemoreceptor depression (carotid and aortic bodies); mu, delta, kappa opiate receptors agonism; a adrenergic receptors agonism/antagonism; and skeletal muscle-acetylcholine-(Ach) receptor activation; as may be needed to optimize rapidity and effectiveness of opioid reversal and to reduce mortality from F/FA related overdose, or as needed for prophylaxis against exposure. Specifically, the treatment for F/FA overdose and toxic exposure involves prevention of and/or reversal of laryngospasm and upper airway effects and chest wall and diaphragm rigidity that appear to be unique to F/FAs as mentioned previously.

Another aspect provided herein deals with overdose due to opiates/opioids in combination with one or more stimulants. In examples of this embodiment, a formulation includes an alpha adrenergic receptor antagonist and a mu opioid receptor antagonist (e.g. naloxone, naltrexone, nalmefene) to concurrently antagonize respiratory depression, FIMR, FIRE syndrome and the cardiovascular effects of each of these drugs (e.g. stimulants and fentanyl/fentanyl analogues).

Prior to this disclosure, no prophylaxis agents or reversal agents that directly treat this combination of drug overdose have existed outside of conventional treatment with a mu or opioid receptor antagonist or a vasoactive agent to limit vascular effects. This disclosure addresses this problem by using similar conceptual technology as the provided immediate reversal agents, but utilizing a different mode of timing and duration to create long-acting or extended—release prophylaxis agents that address the F/FAs side effect profile.

This disclosure recognizes and addresses the necessity for formulation development that specifically addresses the needs, skill sets, and medical training level of different untrained users and a range of medical practitioners.

The immediate reversal formula for F/FA and stimulant overdose or toxic exposure is formulated, in various embodiments, as either a non-prescription, minimal training-required version or a more sophisticated, prescription-only version for a provider who is medically trained in airway (AW) and hemodynamics management. Examples of the minimal or untrained user formulations contain a mu opioid receptor antagonist or another opioid receptor (mu, kappa, and/or delta receptor subtypes) antagonist, an anticholinergic agent (muscarinic antagonist e.g. atropine) or muscarinic agonist (e.g. pilocarpine M3), an α-1 adrenergic antagonist, a selective α-1D adrenergic antagonist and/or an α-2 adrenergic agonist or additionally a beta blocker (e.g. Atenolol, esmolol, metoprolol). Examples of the formulations for a provider medically trained in AW and hemodynamics management may contain a combination of a mu, kappa and/or delta receptor antagonists, an α-1 or α-1D adrenergic antagonist, an anti-cholinergic or M3 agonist to prevent bradycardia and/or upper airway effects, and a rapid acting muscle relaxant/paralytic (such as succinylcholine or rocuronium) in a dose range that relaxes skeletal muscle without causing airway (AW) compromise or to fully secure the AW. Optionally, if conventional naloxone fails and one of the multi-drug formulations in this disclosure fail to reverse WCS, a full dose of muscle paralytic can be administered along with endotracheal intubation to secure the airway; these actions are taken by personnel medically trained to do so. For instance, if the analogue is so potent that its effect is not overcome by the compounds listed here, a failsafe is to secure the airway with a full dose of a muscle paralytic, intubate the patient, and ventilate with 100% oxygen. It is anticipated that most of the current F/FAs will be treated with the compounds listed here, some of which are combined with muscle paralytics.

Provided herein are myriad compositions and methods for treating multiple levels of mechanism of action (MOA) of opiate receptor and alpha 1 adrenergic receptor activation or binding in different organ systems of the body, such as the vascular system, heart, different brain regions, receptor cells in aorta and carotids and pontine and medullary motor nuclei controlling the AW and respiratory muscles of the chest wall and abdomen.

Additional details regarding various embodiments are provided further below.

### BRIEF DESCRIPTION OF THE DRAWING

FIG. 1 Mode of Action (MOA) of Fentanyl at Locus Coeruleus and Medulla: Among humans, the clinical model of FIRE syndrome demonstrates the significant involvement of the larynx and vocal cords and laryngospasm is the key feature of FIRE syndrome. In summary, FIRE syndrome is a syndrome that includes laryngospasm, respiratory muscle rigidity/contraction, cardiovascular compromise and a concurrent decline in hepatic metabolism. Each component can be explained as a consequence of alpha-1 adrenergic, noradrenergic and cholinergic receptor activity in addition to mu opioid receptor activation. The path begins with increased norepinephrine in the LC by fentanyl (indirectly from reuptake inhibition at catecholamine transporter levels), followed by activation and/or inhibition of noradrenergic and cholinergic signal paths through the CNS and terminates on laryngeal muscles resulting in vocal cord closure (laryngospasm). The mechanism has several pathways: A.1). F/FA activation of the LC by systemic administration in peripheral vein or lungs. F/FAs enter the CNS via the carotid and vertebral arteries, cross the blood brain barrier and bind mu opioid receptors on GABA interneurons in the LC, which inhibits GABA cell firing, prevents GABA release and results in increased release of norepinephrine from presynaptic terminals of the LC. As norepinephrine levels increase in the synapse from these combined mechanisms, norepinephrine binds alpha-1 adrenergic receptors on the post synaptic terminal and increases the noradrenergic outflow signals from the LC to several sites that ultimately impact muscle tone in the chest wall, abdomen, diaphragm and laryngeal muscles. Noradrenergic signals from the LC can increase muscle contractility via coerulospinal sympathetic fibers to spinal motor neurons, superior cervical and other sympathetic ganglia (T1-L2) that terminate on skeletal muscle (e.g. chest wall, diaphragm, laryngeal muscles), or via noradrenergic fibers to respiratory motor centers in the medulla (Dorsal respiratory group-DRG and Ventral respiratory group-VRG) and motor efferents in the nucleus ambiguus vagal nuclei controlling innervation of the intrinsic laryngeal muscles. A.2) Vagus nerve: The vagus nerve originating in the nucleus ambiguus of the medulla exits the brain to supply the intrinsic striated muscles of the larynx, providing bilateral motor innervation via the external branch of the superior laryngeal and the recurrent laryngeal nerves. Cricothyroid muscle is the sole tensor of vocal cords innervated via the superior laryngeal nerve. All other larynx muscles are innervated by the recurrent laryngeal nerve (e.g. lateral cricoarytenoid (LCA), posterior cricoarytenoid (PCA) and the thyroarytenoid (TA) muscles. Importantly, the PCA muscles are the sole abductors of the vocal cords and the LCA and TA muscles adduct the vocal cords. Dominant vagal parasympathetic tone to the intrinsic muscles of the larynx allows the abduction via the PCA and keeps glottis/airway open for relaxed inspiration and expiration. A.3) Sympathetic fibers in the vagus nerve originating from the LC contribute innervation directly and indirectly to the intrinsic muscles of the larynx through several pathways coerulospinal fibers terminate in spinal motor neurons that then relay noradrenergic efferent signals from the CNS to sympathetic ganglia and superior cervical sympathetic ganglion (SCG) that supplies the head and neck with sympathetic innervation. Specifically, the SCG provides sympathetic fibers to the terminal branches of the vagus nerve that serve as the sole innervation of the intrinsic muscles of the larynx. Increased sympathetic outflow from the LC and offers a plausible mechanism whereby norepinephrine can rapidly activate vocal cord adductors to cause severe laryngospasm, particularly in a system where cholinergic/parasympathetic tone is diminished or compromised (e.g. anticholinergic drug effects). A.4) Cholinergic innervation of the VC/larynx: The LC controls autonomic nuclei in the medulla, specifically the nucleus ambiguus, which gives rise to the efferent motor fibers of the vagus nerve. Alternatively, fentanyl can directly bind cholinergic nuclei in the nucleus ambiguus. acting as a selective M3 antagonist and could facilitate selective isolation of M1 and M2 receptors for ACh, binding, resulting in increased relaxation of laryngeal abductor muscles and diminished opposition to sympathetically mediated laryngeal adductor contraction. A.5) Diaphragm, Abdomen and Thoracic wall innervation: the main muscles of respiration include the diaphragm, intercostals and abdominal wall muscles. Diaphragm receives its motor nerve impulses from the medullary centers via the phrenic nerve and sympathetic nerve fibers from the cervical sympathetic chain. The intercostal muscles are stimulated by way of cervical, thoracic and lumbar motor nerves and spinal motor neurons that terminate on skeletal muscles of the thorax and abdomen. A.6) Medulla DRG VRG: Respiration increases or decreases via afferent signals from the vagus and glossopharyngeal nerves via peripheral chemo and mechanical receptors and mechanical changes in the lung and airway. These signals return to respiratory centers in the pons, medulla (i.e., dorsal respiratory group [DRG]) and ventral respiratory group (VRG). DRG controls initiation of inspiration via motor nerves to diaphragm and external intercostal muscles. VRG contains inspiratory and expiratory neurons and controls laryngeal/ pharyngeal muscles, diaphragm, abdominal and intercostals. Additionally, vagus nerve fibers contain mu opioid receptors that innervate stretch receptors in lung and can send afferent signals to vagal nuclei when activated by opioid binding causing a cessation of inspiratory drive to prevent over-inflation of the lung via inhibitory signals to the DRG to cease further inspiration, and may include VRG-mediated closure of the vocal cords. A.7) Cardiac Function in FIRE syndrome: Cardiac output can be inhibited by vagus nerve activation in the nucleus ambiguus. Fentanyl binds mu opioid receptors in vagal nuclei and GABA interneurons of the nucleus ambiguus, and can cause severe bradycardia and decreased cardiac output, with direct consequences for cerebral and hepatic perfusion pressures. As noted herein, fentanyl also antagonizes alpha 1 adrenergic receptor subtypes in a rank order of 1B>1A »1 D and may have significant consequences on cardiac function via selective distributions of these receptors in coronary arteries. These proposed cardiac mechanisms may explain the rapid onset of vascular, hepatic and CNS effects described in public health/autopsy data including: 1) rapid onset of cyanosis, 2) immediate loss of consciousness (central thalamocortical inhibition and/or decreased cerebral perfusion decrease) and 3) decreased hepatic metabolism. A.8) Hepatic Function in FIRE syndrome: By decreasing cardiac output, high dose fentanyl can secondarily cause decreased hepatic artery perfusion and/ or potentially decreased cerebral perfusion, resulting in a significant malfunction in fentanyl metabolism and a rapid loss of consciousness.

The proposed mode of action for stimulants combined with fentanyl/ fentanyl analogues is that each drug acts to increase norepinephrine levels in the CNS and periphery by direct and indirect action on transporter molecules, intracellular vesicles and by selective binding of alpha 1 adrenergic receptors in the case of F/FAs, all serving to reinforce the life-threatening effects of overdose with each of these drugs singly and in combination.

FIGS. 2A, 2B illustrate binding of compounds at Adr1A: At the alpha 1A receptor, fentanyl has comparable affinity, as seen by Ki values, as NE. Carfentanil, in contrast has a 2 fold greater affinity at the 1A compared to fentanyl and NE. By comparison prazosin and tamsulosin each have BA in the subnanomolar (<1 nM) range at all subtypes and BA that is 4-5 orders of magnitude greater than either fentanyl, carfentanil or NE. Additionally, prazosin and tamsulosin have a 4-6 orders of magnitude greater BA at each subtype over either fentanyl or NE.

FIGS. 3A, 3B illustrate binding of compounds at Adr1B: At the alpha 1B receptor, fentanyl has comparable affinity as carfentanil, as seen by Ki values, and in contrast has a 2 fold greater affinity at the 1B compared to NE. By comparison prazosin and tamsulosin each have BA in the subnanomolar (<1 nM) range at all subtypes and BA that is 4-5 orders of magnitude greater than either fentanyl, carfentanil or NE. Additionally, prazosin and tamsulosin have a 4-5 orders of magnitude greater BA at each subtype over either fentanyl, carfentanil or NE.

FIGS. 4A, 4B illustrate binding of compounds at Adr1D: At the alpha 1D receptor, fentanyl and carfentanil have comparable affinity, as seen by Ki values. NE, in contrast has a ˜25-30 fold greater affinity at the 1D compared to carfentanil and fentanyl, respectively. Notably, the 1D subtype is where NE demonstrates its greatest binding affinity. By comparison prazosin and tamsulosin each have BA in the subnanomolar (<1 nM) range at all subtypes and BA that is 4-6 orders of magnitude greater than either fentanyl, carfentanil or NE. Additionally, prazosin and tamsulosin have a 4-6 orders of magnitude greater BA at each subtype over either fentanyl, carfentanil or NE.

FIGS. 5A-5D are a series of graphs showing measurements taken using PhysioSuite, during an anesthesia comparison in the animal model described in Example 10. FIG. 5A shows oxygen saturation; FIG. 5B shows Heart rate; FIG. 5C shows perfusion rate; and FIG. 5D shows body temperature; each includes charts of baseline (left panel), in the presence of glycopyrrolate (middle panel), and before and after administration of fentanyl (right panel). In each graph, open circles represent samples from animals treated with ketamine and xylazine (80 and 8 mg/kg, respectively; n=5-6); and closed squares represent samples from animals treated with urethane and α-chloralose (1200 and 40 mg/kg, respectively; n=4-6). All measurements were taken 1/s, averaged over 15 seconds.

FIGS. 6A-6C illustrate additional measurements taken using PhysioSuite, in the animal model described in Example 10. FIG. 6A shows the oxygen saturation in animals treated with fentanyl. FIG. 6B is a graph showing the heartrate of the same animals across the same time course. In both, open circles represent samples from animals treated with ketamine and xylazine (80 and 8 mg/kg, respectively); and closed squares represent samples from animals treated with urethane and a-chloralose (1200 and 40 mg/kg, respectively). FIG. 6C shows the number of animals sampled for each of the indicated timepoints. All measurements were taken 1/s, averaged over 15 seconds.

FIGS. 7A-7B are photographs of rat vocal cords before (FIG. 7A) and 15 seconds after (FIG. 7B) administration of fentanyl to a rat, in the animal model described herein.

### DETAILED DESCRIPTION

The present disclosure takes advantage of combined and, in some cases, synergistic effect(s) between mu and/or opioid receptor antagonists, cholinergic agents and one or more of a-adrenergic agonists/antagonists, anticholinergics and other vasoactive agents to provide novel combinations having utility in the reversal of or prophylaxis against opioid/opiate effects (e.g. F/FAs and morphine derived alkaloids). These compounds are designed to treat respiratory depression from conventional morphine derived opiates and/or fentanyl and fentanyl analogue (F/FA) induced respiratory muscle rigidity (FIRMR), FIRE syndrome and vasoactive effects from both F/FAs and stimulants. Different and specific formulations described here can be used as reversal drugs, prophylaxis against F/FA and stimulant environmental exposure, and for polysubstance exposure reversal (e.g. F/FAs and/or morphine derivatives combined with benzodiazepines). Embodiments of the described methods involve identification of treatment individuals or groups, treatment by clinical presentation of individual subjects (for instance mammalian subjects, such as humans), and provision of treatment formulation(s) as per the expected or known skill set of the user. Overall, these are largely reiterations of how to use the focus of the herein described technology, which is the compositions and compounds described.

TABLE 1: Base Dose Compounds (BDC) and Treatment (provided in four parts).

TABLE 1
BASE DOSE COMPOUNDS (BDC) and TREATMENT ALGORITHMS

| | | | | Optional | | |
|---|---|---|---|---|---|---|
| | | | | Vasoactive/ | | |
| | | | ALPHA-1 | Vasodilator/ | ANTICHOLINERGICS | |
| DRUG CLASSES* | | MU | S/NS | Vasopressor | (AC) | PARALYTICS |
| MODE | IMMEDIATE REVERSAL | | | | | |
| | FENTANYL & FENTANYL ANALOGUES | | | | | |
| | NON-MEDICAL | Naloxone | NS- Prazosin | See ¥ below | N/A | N/A |
| | | (1-4 mg) 1-4 | (0.2-0.5 mg) | | | |
| | | doses of | and/or | | | |
| | | BDC or | S- Tamsulosin | | | |
| | | Nalmefene | (0.2-0.4 mg) | | | |
| | | (0.5 mg) 1-4 | 1-4 doses of | | | |
| | | doses of | BDC | | | |
| | | BDC | | | | |
| | MEDICAL W/O | Naloxone | NS- Prazosin | See ¥ below | Atropine (0.5 mg) | N/A |
| | AIRWAY W/ | (1-4 mg) 1-4 | (0.2-0.5 mg) | | and/or Glycopyrrolate | |
| | Hemodynamic | doses of | and/or | | (0.2 mg) 1-4 | |
| | Monitoring | BDC or | S- Tamsulosin | | doses of BDC | |
| | Available | Nalmefene | (0.2-0.4 mg) | | *Avoid if subject is | |
| | | (0.5 mg) 1-4 | 1-4 doses of | | normocardiac; tachycardic or | |
| | | doses of | BDC | | with cardiac arrhythmia | |
| | | BDC | | | | |
| | MEDICAL W | Naloxone | NS- Prazosin | See ¥ below | Atropine (0.5 mg; | Succinylcholine |
| | AIRWAY W/ | (1-4 mg) 1-4 | (0.2-0.5 mg) | | and/or | (1-3 mg) 1-4 |
| | Airway Equipment | doses of | and/or | | Glcopyrrolate (0.2 mg) | doses of |
| | Available | BDC or | S- Tamsulosin | | 1-4 doses of BDC | BDC |
| | | Nalmefene | (0.2-0.4 mg) | | *Avoid if subject is | |
| | | (0.5 mg) 1-4 | 1-4 doses of | | normocardiac, tachycardic or | |
| | | doses of | BDC | | with cardiac arrhythmia | |
| | | BDC | | | | |
| | CLINICAL PRESENTATION † | | | | | |
| | 1) Suspected | Naloxone | NS- Prazosin | See ¥ below | Atropine (0.5 mg) | +/− |
| | opioid and | (1-4 mg) 1-4 | (0.2-0.5 mg) | and/or Beta | and/or | |
| | stimulant OD, | doses of | and/or | Blocker | Glycopyrrlate (0.2 mg) | |
| | unresponsive | BDC | S- Tamsulosin | (BETAB)(e.g. | 1-4 doses of BDC*Avoid if | |
| | patient with rapid, | Nalmefene | (0.2-0.4 mg) | esmolol, | subject is normocardiac, | |
| | bounding pulse | (0.5 mg) 1-4 | 1-4 doses of | atenolol | tachycardic or with cardiac | |
| | indicating high | doses of | BDC | metoprolol), | arrhythmia | |
| | BP: | BDC | | | | |
| | 2) Suspected | Naloxone | NS- Prazosin | See ¥ below | Atropine (0.5 mg) | Succinylcholine |
| | Opioid OD with | (1-4 mg) 1-4 | (0.2-0.5 mg) | Avoid | and/or | (1-3 mg) 1-4 |
| | prominent | doses of | and/or | application of | Glycopyrrlate (0.2 mg) | doses of |
| | RIGIDITY or | BDC | S- Tamsulosin | Vasopressor | 1-4 doses of BDC | BDC |
| | severe | Nalmefene | (0.2-0.4 mg) | agents due to | To maximize inhibition of | |
| | laryngospasm | (0.5 mg) 1-4 | 1-4 doses of | hypertension or | vagal tone in the CNS/LC | |
| | Hypertensive | doses of | BDC | tachycardia. | *Avoid if subject is | |
| | crisis | BDC | | | normocardiac, tachycardic or | |
| | | | | | with cardiac arrhythmia | |
| | 3) Suspected | Naloxone | NS- Prazosin | Phenylephrine | Atropine (0.5 mg) | +/− |
| | Opioid OD is | (1-4 mg) 1-4 | (0.2-0.5 mg) | (58-200 mg) | and/or | |
| | pulseless-NO | doses of | and/or | 1-4 doses | Glycopyrrlate (0.2 mg) | |
| | PULSE and LOW | BDC | S- Tamsulosin | of BDC | 1-4 doses of BDC | |
| | BP pressure | Nalmefene | (0.2-0.4 mg) | Ephedrine | | |
| | UNCERTAIN | (0.5 mg) 1-4 | 1-4 doses of | (5-10 mg) | | |
| | PRESENTATION | doses of | BDC | 1-4 doses | | |
| | | BDC | | of BDC | | |
| | | | | Epinephrine**** | | |
| | POLYSUBSTANCE | | | | | |
| | NON-MEDICAL | Naloxone | NS- Prazosin | See ¥ below | N/A | N/A |
| | | (1-6 mg) 1-6 | (0.2-0.5 mg) | Avoid | | |
| | | doses of | and/or | application of | | |
| | | BDC | S- Tamsulosin | Vasopressor | | |
| | | | (0.2-0.4 mg) | agents due to | | |
| | | | 1-4 doses of | hypertension or | | |
| | | | BDC | tachycardia. | | |
| | MEDICAL W/O | Naloxone | NS- Prazosin | See ¥ below | N/A due to | N/A |
| | AIRWAY W/ | (1-6 mg) 1-6 | (0.2-0.5 mg) | | seizure risk. | |
| | Hemodynamic | doses of | and/or | | | |
| | Monitoring | BDC | S- Tamsulosin | | | |
| | avail. | Nalmefene | (0.2-0.4 mg) | | | |
| | | (0.5 mg) 1-4 | 1-4 doses of | | | |
| | | doses of | BDC | | | |
| | | BDC | | | | |
| | MEDICAL W | Naloxone | NS- Prazosin | See ¥ below | N/A due to | Succinylcholine |
| | AIRWAY | (1-6 mg) 1-6 | (0.2-0.5 mg) | N/A on | seizure risk. | (1-3 mg) 1-4 |
| | W/ AW EQUIP | doses of | and/or | Vasopressors | | doses of |
| | AVAIL. | BDC | S- Tamsulosin | agents due to | | BDC |
| | | Nalmefene | (0.2-0.4 mg) | hypertension, | | |
| | | (0.5 mg) 1-4 | 1-4 doses of | tachycardia | | |
| | | doses of | BDC | and/or due to | | |
| | | BDC | | seizure risk. | | |
| | PROPHYLAXIS | | | | | |
| | IV USER | Naltrexone | NS- Prazosin | SEE | Atropine | N/A |
| | | (25-50 mg) | (0.2-0.5 mg) | “LEGEND* | (0.5 mg) and/or | |
| | | Nalmefene | and/or | | Glycopyrrolate | |
| | | (0.5 mg) 1-4 | S- Tamsulosin | | (0.1-0.2 mg/hour | |
| | | doses of | (0.2-0.4 mg) | | in a time | |
| | | BDC | | | release PO tad) | |
| | | | | | Scopolamine | |
| | | | | | *Avoid if | |
| | | | | | subject is | |
| | | | | | normocardiac, | |
| | | | | | tachycardic or | |
| | | | | | with cardiac | |
| | | | | | arrhythmia | |
| | 1^{st} RESPONDER | Naltrexone | NS- Prazosin | SEE | N/A | |
| | | (25-50 mg) | (0.2-0.5 mg) | “LEGEND” | | |
| | | | and/or | | | |
| | | | S- Tamsuiosin | | | |
| | | | (0.2-0.4 mg) | | | |

| | Optional | | |
|---|---|---|---|
| | | | COMBO |
| | RESPIRATORY | GABA | AGENTS |
| | ACCELERANTS | COMPLEX | (alpha-1 + |
| DRUG CLASSES* | (RA) | ANTAGONISTS | AC) |

| | |
|---|---|
| MODE | IMMEDIATE REVERSAL |
| | FENTANYL & FENTANYL ANALOGUES |

| NON-MEDICAL | N/A due to | N/A | |
|---|---|---|---|
| | seizure risk. | | |
| MEDICAL W/O | N/A due to | N/A | Droperidol § |
| AIRWAY W/ | seizure risk. | | (0.5 mg) 1-4 |
| Hemodynamic | | | doses of |
| Monitoring | | | BDC |
| Available | | | |
| MEDICAL W | N/A due to | N/A | Droperidol § |
| AIRWAY W/ | seizure risk. | | (0.5 mg) 1-4 |
| Airway Equipment | | | doses of |
| Available | | | BDC |
| CLINICAL PRESENTATION † | | | |
| 1) Suspected | +/− | N/A | |
| opioid and | | | |
| stimulant OD, | | | |
| unresponsive | | | |
| patient with rapid, | | | |
| bounding pulse | | | |
| indicating high | | | |
| BP: | | | |
| 2) Suspected | +/− | N/A | Droperidol § |
| Opioid OD with | | | (0.5 mg) 1-4 |
| prominent | | | doses of |
| RIGIDITY or | | | BDC |
| severe | | | |
| laryngospasm | | | |
| Hypertensive | | | |
| crisis | | | |
| 3) Suspected | +/− | N/A | |
| Opioid OD is | | | |
| pulseless-NO | | | |
| PULSE and LOW | | | |
| BP pressure | | | |
| UNCERTAIN | | | |
| PRESENTATION | | | |
| POLYSUBSTANCE | | | |
| NON-MEDICAL | N/A | N/A | |
| MEDICAL W/O | N/A due to | Flumazenil | |
| AIRWAY W/ | seizure risk. | (0.2 mg) ** | |
| Hemodynamic | | Dilantin | |
| Monitoring avail. | | (50 mg) *** | |
| MEDICAL W | N/A due to | Flumazenil | |
| AIRWAY | seizure risk. | (0.2 mg) ** | |
| W/ AW EQUIP | | Dilantin | |
| AVAIL. | | (50 mg) *** | |
| PROPHYLAXIS | | | |
| IV USER | N/A | N/A | Droperidol § |
| | | | (0.5 mg) 1-4 |
| | | | doses of |
| | | | BDC |
| 1^{st} RESPONDER | N/A | N/A | N/A |

With regard to Table 1:
† Uncertain Presentation: In the event that the medical provider is uncertain of physiologic or clinical presentation in “Suspected Opioid Overdose with Stimulants”, use the baseline “Non-Medical Provider” dosing kit until vital signs are apparent and directional, then follow algorithm as above in clinical scenarios 1-4.
*All of these drug classes, with the exception of the A1ARAs prazosin and tamsulosin (see “A1ARA
IV/IN/IM formulation protocol”), are available as IV formulations and therefore can be easily converted to nasal dosing regimens, which are similar in potency and concentration, if not the same, and will be concentratable in a nasal (e.g., insufflated), INH, IV, IM, IO and intraocular (IOC) formulation.
** Dilantin and Flumazenil are given in a ratio of 50 mg/0.2 mg as a prophylaxis against the risk or occurrence or seizures due to rapid benzodiazepine reversal in drug overdoses involving individuals with regular or habitual use of benzodiazepines.
*** In the event of “status epilepticus” induced by rapid reversal of benzodiazepine overdose or from stimulant overdose, a conversion to use of separate baseline reversal drug (e.g. MU + NS-A1ARA + S-A1ARA) with IV Dilantin (5-15 mg/kg) with infusion rate NTE 50 mg/min due to risk of cardiac arrhythmia.
****Epinephrine is to be used with caution in individuals with F/FAs and stimulant(s) overdose due to the direct and potent activity of Epinephrine and Noradrenaline at the LC and FIMR related circuitry and to avoid SSOIVE. However, should this be the initial presentation in “ Suspected Opioid Overdose”, the medical practitioner should use their discretion to follow best practices and go directly to the most current ACLS cardiac arrhythmia treatment algorithms with the possible addition of the “Baseline formulation” for FIMR reversal.
§ Droperidol (combined alpha-1 adrenergic antagonist and anticholinergic- AARA-AC) can be administered in a dose range of 0.01- 0.25 mg/kg IV, IM or IN. Dosing at higher ranges is known to be associated with increased risk of cardiac arrhythmias and is contraindicated in prolonged QTc intervals (SEE black box warning label), however is rare in occurrence. Initial doses of up to 2.5 mg are well tolerated with additional doses of 0.5-1.25 mg may be administered if benefit of F/FA overdose or toxic exposure reversal outweighs potential risk of upper dose range.
♦ Pilocarpine (M3/muscarinic agonist) is an example of a muscarinic agonist recommended for use in events where it may be of benefit in F/FA overdose or toxic exposure and reversal of F/FA outweighs potential risk of upper dose range. (NOTE: Broad-acting muscarinic agents may in some cases provoke laryngospasm; development of M3-specific agonist may overcome this).
¥ Esmolol dosage: Acute MI or Hypertensive Crisis: 25-200 mcg/kg/min IV infusion (e.g. 70 kg: 100 mcg/kg/min), 5-25 mg IV Bolus (e.g. 1-5 doses). Atenolol dosage: Acute MI or Hypertensive Crisis: 5 mg IV Bolus over 5” (e.g. repeat 1-5 doses), for prophylaxis 25-50 mg PO QD. Metoprolol dosage: Acute MI or Hypertensive Crisis: 5 mg IV Bolus over 5″ (e.g. repeat 1-5 doses), for prophylaxis 25-100 mg PO QD.

The following sections describe information and steps to support therapeutically effective treatments for preventing or reversing one or more effect(s) of opioid(s) or opiate(s) in combination with one or more stimulants in an individual (for instance, to treat or prevent accidental overdose or to provide prophylaxis against environmental exposure). The sections include: (i) Abbreviations & Exemplary Definitions; (ii) Fentanyl and its Effects; (iii) Proposed Mode(s) of Action; (iv) Therapeutic Compounds (including subsections (a) al-Adrenergic Receptor Antagonists; (b) Mu and/or opioid receptor subtype antagonist; (c) paralytics/muscle relaxants; (d) α2-adrenergic receptor agonist; and (e) GABA/benzodiazepine receptor complex antagonists; (v) Compositions for Methods of Use; (vi) Methods of Use; (vii) Kits; (viii) Exemplary Embodiments; (ix) Examples; and (x) Closing Paragraphs.

### (I) ABBREVIATIONS & EXEMPLARY DEFINITIONS

A1ARs α1 Adrenergic receptors

A1ARAs α1 Adrenergic receptors antagonists

A1-A α1-A Adrenergic receptors antagonists- subtype specific antagonists

A1-B α1-B Adrenergic receptors antagonists- subtype specific antagonists

A1-D α1-D Adrenergic receptors antagonists- subtype specific antagonists

AARA α adrenergic receptor antagonist

AC anticholinergic drug (M1-M5 antagonists)

AW airway

Beta B beta blocker (β blocker)

BP blood pressure

C cholinergic drug (M1- M5 agonist, Nicotinic receptor agonist)

D5W 5% dextrose in sterile water

FIMR fentanyl induced muscle rigidity

FIRE fentanyl induced respiratory effects

FIRMR fentanyl induced respiratory muscle rigidity

FIVE fentanyl induced vascular effects

F/FAs fentanyl and fentanyl analogues

HR heart rate

IRMAW Immediate Reversal Medical AW

IRMnAW Immediate Reversal Medical No AW

IRNM Immediate Reversal Non-Medical

M muscarinic receptors

M3 specific muscarinic receptor

M1-M5 muscarinic receptors,

NIC nicotinic receptors

PAOU Prophylaxis for Active Opioid User

PFR Prophylaxis for First Responders

PILO Pilocarpine

Poly Polysubstance

SSOIVE Stimulant and Synthetic Opioid Induced Vascular Events

WCS wooden chest syndrome (combined FIRMR and laryngospasm)

VC vocal cords

The term “synergistic” as used herein means that the effect achieved with the compounds used together is greater than the sum of the effects that result from using the compounds separately. For example some of the compounds will include: mu or opioid receptor (mu, kappa, delta receptor subtypes) antagonists/ agonists and α adrenergic antagonists, α adrenergic agonists, respiratory accelerants, vasoactive agents, anticholinergics, cholinergic agents (muscarinic receptor antagonist/ anticholinergic, M3 receptor agonist or a nicotinic receptor general or selective agonist) and/or paralytics described herein are sometimes referred to herein as the “synergistic ingredients” or the “synergistic compounds.”

The degree of synergism of the combinations of the herein disclosed technology can be analyzed by estimation of a combination index (Fu et aL, Synergy, 3(3):15-30, 2016). In some embodiments, the term “synergistic combinations” refers herein to combinations characterized by a combination index >1.

The term “synergistic combinations” refers herein to combinations characterized by an a parameter that is positive and for which the 95% confidence interval does not cross zero. In the practice of the present invention, the synergistic combinations preferably are characterized by an a interaction parameter that is greater than about 2, and more preferably by an a parameter that is greater than about 4.

The term “pharmaceutically acceptable derivative” is used herein to denote any pharmaceutically or pharmacologically acceptable salt, ester, amide or salt of such ester or amide of a synergistic compound according to the invention.

A “pharmaceutically acceptable salt” is intended to mean a salt that retains the biological effectiveness of the free acids and bases of the specified compound and that is not biologically or otherwise undesirable. Examples of pharmaceutically acceptable salts include but are not limited to sulfates, pyrosulfates, bisulfates, sulfites, bisulfites, phosphates, monohydrogenphosphates, dihydrogen-phosphates, metaphosphates, pyrophosphates, chlorides, bromides, iodides, acetates, propionates, decanoates, caprylates, acrylates, formates, isobutyrates, caprotes, heptanoates, propioltes, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxy-benzoates, phthalates, sulfonates, sulfamates, xylenesulfonates, phenylacetates, phenylpropionates, phenylbutyrates, citrates, lactates, gamma-hydroxybutyrates, glycollates, tartrates, methanesulfonates, propanesulfonates, naphthalene-1-sulfonates, naphthalene-2-sulfonates, and mandelates.

“Analogs” is intended to mean compounds derived from a particular parent compound by straightforward substitutions that do not result in a substantial (i.e. more than 100×) loss in the biological activity of the parent compound, where such substitutions are modifications well-known to those skilled in the art, e.g., esterification, replacement of hydrogen by halogen, replacement of alkoxy by alkyl, replacement of alkyl by alkoxy, etc.

“Therapeutically effective combination” means an amount of a compound herein described combination that, when administered to a patient in need of treatment, is sufficient to effect treatment for the disease condition alleviated by the (optionally, synergistic) combination. In the immediate reversal scenario, several metrics are significant in monitoring for successful treatment. A combination drug is beneficial as no single agent treats all three of the active receptor sites engaged by fentanyl and other F/FAs: mu opioid receptors, muscarinic and alpha adrenergic receptors. Particularly, naloxone has a minimal impact on the effects of F on VC and laryngeal muscles/laryngospasm at doses relevant or safe to humans (e.g. naloxone effect at >0.8 mg/kg in rat model) (Willette et al., J Pharmacol Methods 17:15-25, 1987; Willette et al., Euro J Pharmacology 80:57-63, 1982; Willette & Sapru, Euro J Pharmacology 78:61-70, 1982).

Several broad categories of therapeutically effects exist, including:

1) Attenuation or Resolution of FIRE syndrome and FIRMR: measured by a reduction, elimination or inhibition of chest wall rigidity, diaphragm rigidity, laryngospasm with return of airway patency and either easy flow of oxygen and ventilation with assisted ventilation or the return of spontaneous respiration with adequate respiratory rate and tidal volume to maintain oxygenation (e.g. Oxygen saturation of >94% by pulse oximetry, Arterial Blood gas-ABG with P-arterial O₂of >80 mmHg pressure of oxygen in the blood PaO₂ETCO₂<40).

2) Return of consciousness and able to follow commands with Glasgow Coma scale score of >12 (8=comatose but responsive to painful stimuli, 3=unresponsive to all stimuli).

3) Hemodynamic parameters adequate to maintain cerebral and coronary perfusion; usually Systolic BP>90 mmHg and <160 mmHg, and Diastolic BP>50 mmHg and <100 mmHg, HR>50 BPM (e.g., resolution of Hypertension, Hypotension, Tachycardia or Bradycardia).

4) Similar or same parameters can be used for prophylaxis users. The prophylaxis agents essentially use a pre-emptive blockade /antagonism of Mu and Alpha adrenergic receptors to increase the dose tolerance and resistance to FIRE syndrome and SSOIVE upon exposure. The agents technically cause a R shift of the dose response curve for FIRE syndrome and SSOIVE and thus either inhibit or delay the onset of effects of F/FA exposure or allow for a higher level of exposure before effects occur.

Amounts of each of these components present in a therapeutically effective combination may not be therapeutically effective when administered singly. Use of the combination is important because no single agent treats all three of the active receptor sites engaged by fentanyl and other F/FAs, notably mu opioid receptors, muscarinic and alpha adrenergic receptors. For instance, naloxone has a minimal impact on the effects of F on VC and laryngeal muscles/laryngospasm as noted above in doses relevant to or safe for humans (Willette et al., J Pharmacol Methods 17:15-25, 1987; Willette et al., European Journal of Pharmacology 80:57-63, 1982; Willette & Sapru, European Journal of Pharmacology 78:61-70, 1982) The amount of a given combination that will be therapeutically effective will vary depending on factors such as the particular combination employed, the particular form of opioid/opiate exposure, the treatment history of the patient, the age and health of the patient, and other factors.

An “opiate” is a drug naturally extracted or directly derived from the opium poppy plant. Examples of opiates include heroin, morphine, hydromorphone and codeine. The term opioid is broader; it includes opiates and also any substance, natural, semi-synthetic or synthetic, that binds to the brain's opioid receptors—the parts of the brain responsible for controlling pain, reward and addictive behaviors. Examples of opioids include fentanyl, sufentanil, alfentanil, remifentanil, carfentanil, oxycodone, oxycontin, hydrocodone, hydromorphone, oxymorphone, meperidine, tapentadol and methadone. There are numerous fentanyl analogues and synthetic opioid analogs and the list here is not meant to be exhaustive, but demonstrative of molecules in this class which act as agonists at opioid receptor subtypes (e.g. Mu, Delta, Kappa) in various selective and non-selective combinations.

“Stimulant” (sometimes referred to as “psychostimulants”) refers to a class of compounds or drugs that increase sympathetic and/or catecholamine and/or monoamine neurotransmitter activity in the central or peripheral nervous systems and/or have sympathomimetic effects by binding to adrenergic receptors as agonists, selective antagonists or by facilitating release of sympathetic neurotransmitters by binding transporter molecules (e.g. dopamine-DAT, norepinephrine-NET, epinephrine) or transport vesicles (e.g. vesicular monoamine transporters- VMAT, VMAT2) or by inhibiting catecholamine/monoamine degradation enzymes such as monoamine oxidase. The term stimulant as it is used in this document refers specifically to drugs such as methamphetamine or cocaine that have sympathomimetic effects which increase the availability and/or release of catecholamines (e.g. norepinephrine) through the various mechanisms listed above and increase the availability of these catecholamines and/or monoamines for binding with alpha 1 or alpha 2 adrenergic or beta 1 or beta 2 adrenergic receptors and/or subtypes of the these alpha and beta receptors, in the mammalian sympathetic, central and peripheral nervous systems or tissues and organs innervated by these sympathetic systems. When stimulants as described here are used in combination with F/FAs, the combination of effects of each of these classes of drugs overlaps in a fashion that enhances these sympathomimetic mechanisms to devastating and lethal effect. In addition to methamphetamine and cocaine, the category of stimulants also includes: amphetamine, methylphenidate (Ritalin), and amphetamine/dextroamphetamine (Adderall).There are numerous analogues of these stimulants and the list here is not meant to be exhaustive, but demonstrative of molecules in this class which act as sympathomimetics through the mechanisms listed above.

“Treatment” in some instances refers to alleviation or prevention of symptoms of FIRE syndrome, SSOIVE, and respiratory depression in a patient or the improvement of these symptoms in an individual in need of such treatment. However, “treatment” in the context of this disclosure is several fold, depending on the embodiment(s):

1. Immediate reversal of FIRE syndrome, SSOIVE, and respiratory depression: The most basic intervention level (e.g., mu antagonist and AARA) for FIRE syndrome and SSOIVE reversal results from the antagonism or blockade of mu receptors, or opioid receptor (mu, kappa, delta receptor subtypes) antagonist combined with an a adrenergic antagonist/agonist to decrease noradrenergic outflow from the LC triggered either directly or indirectly at mu opioid or a adrenergic receptors by F/FAs and stimulants, resulting from intravenous injection, inhalation, or ingestion, for instance. Additionally beta blocker (e.g. Atenolol, esmolol, metoprolol), and/or a cholinergic agent (muscarinic receptor antagonist/ anticholinergic, M3 receptor agonist or a nicotinic receptor general or selective agonist) may be optionally added to antagonize the potential direct or indirect effects of fentanyl and F/FAs and on muscarinic receptors and nicotinic receptors and/or stimulants on alpha and beta adrenergic receptors. This can be gauged as mentioned previously by either the return and ease of spontaneous respiration or the return of ability to perform assisted ventilation and/or the ability to secure the AW if necessary. Essentially this is a return of AW patency and increase in thoracic compliance (e.g. relaxed chest wall muscles) that allows for oxygen exchange and the reversal of hypoxemia and hypercarbia and can be objectively measured by end-tidal— 0O2 concentrations (ETCO₂), Pulse oximetry (O₂Saturation % difference between oxygenated hemoglobin-Hgb and deoxygenated Hgb) and arterial blood gas concentrations (PaO₂, PaCO₂ in mmHG). Return of level of consciousness (LOC) using the Glasgow Coma Scale as noted above, is also a significant measure of the reversal of is routinely associated with an instantaneous loss of consciousness with return of consciousness as FIMR is wearing off or actively inhibited. Maintain or return of hemodynamic parameters adequate to maintain cerebral and coronary perfusion; usually Systolic BP >90 mmHg and <160 mmHg and Diastolic BP>50 mmHg and <100 mmHg, HR >50 BPM and HR <100 BPM with maintenance of Sinus Rhythm for maintenance of cardiac output and perfusion pressure.

2. Prophylaxis against FIRE syndrome, SSOIVE and respiratory depression: This can be gauged by the either the prevention of FIRE syndrome, SSOIVE and respiratory depression or a reduction in AW and ventilation compromise symptoms and vascular compromise resulting from intravenous injection, inhalation or ingestion etc. with F/FAs and stimulants. Ideally, if the NA outflow from the LC has been previously inactivated by an AARA, which acts to hyperpolarize and inactivate NA neurons in the brainstem and spinal cord and blocks norepinephrine from landing on AARAs, even exceedingly high doses of F/FAs and stimulants may allow the patient to remain asymptomatic or only mildly affected. Additionally a beta blocker (e.g. Atenolol, esmolol, propranolol), may be optionally added to antagonize the potential direct or indirect effects of fentanyl and F/FAs and/or stimulants on alpha and beta adrenergic receptors. The combined effect of a long acting Mu opioid antagonist (e.g. naltrexone, nalmefene) and an alpha 1 adrenergic antagonist are ideal for prophylaxis. In the case of individuals who are affected despite receiving a prophylaxis dose, an immediate reversal dose can be “stacked” on top of the prophylaxis dose to block and or antagonize any of the remaining receptors that might still be available for binding by F/FAs.

3. “Stacking dose”: in the event that an individual has already received prophylaxis dosing, but becomes symptomatic from F/FAs combined with stimulants or stimulants contaminated with F/FAs, additional doses of the immediate reversal agent can be given. In this case it may be recommended to give a modified version of the immediate reversal agent that includes Naloxone, a 1A or 1D subtype selective AARA (e.g., tamsulosin) and a vasoactive agent (e.g., Beta blocker or calcium channel blocker). Additionally a beta blocker (e.g. Atenolol, esmolol, propranolol), may be optionally added to antagonize the potential direct or indirect effects of fentanyl and F/FAs and/or stimulants on alpha and beta adrenergic receptors. Additionally, a cholinergic agent (muscarinic receptor antagonist/ anticholinergic, M3 receptor agonist or a nicotinic receptor general or selective agonist) optionally may be added to antagonize the potential direct or indirect effects of fentanyl and F/FAs on muscarinic receptors and nicotinic receptors in the presentation of significant vagal tone demonstrated clinically as bradycardia (HR <60 BPM). Similar parameters can be used to measure success of reversal as mentioned above in this section.

### (II) FENTANYL AND ITS EFFECTS

First developed by Janssen Pharmaceuticals in the 1950's as a more hemodynamically stable and potent analgesic alternative to morphine and other synthetic opiates, fentanyl and its analogues (FAs) are highly potent, synthetic, mu-opiate receptor agonists with a potency 100-10,000 times greater than morphine or heroin. Despite having a very narrow therapeutic window, the fentanyl family of opioids have been safely used in medicine for over 50 years and to great effect in surgical anesthesia and pain management, when administered by Anesthesiologists and trained medical personnel (Grell et al., Anesth Analg 49(4):523-532, 1970; Streisand et al., Anesthesiology 78(4):629-634, 1993; Bennett et al., Anesthesiology 87(5):1070-1074, 1997; Coruh et al., Chest. 143(4):1145-1146, 2013).

Naloxone, a mu opioid receptor antagonist, is currently the only FDA-approved medication for reversal of opioid overdose and specifically targets respiratory depression induced by opioids. Recent public health reports from major urban areas affected by increasing numbers of overdoses involving fentanyl and its analogues have reported a dramatic rise in the number of naloxone doses needed to reverse the effects of fentanyl (e.g. 2-12 doses of naloxone; Walley et al., Morb Mortal Wkly Rep 66:382-386, 2017; Chou et al., Ann Intern Med 167(12):867-875, 2017). High dose naloxone (e.g. 0.2 mg/kg), and even doses that are two times the normal dose (e.g. 0.0005 mcg/kg)) , regularly precipitate severe cardiac arrhythmias, hemodynamic instability and pulmonary edema in active opioid users, which are all potentially life-threatening (Clarke et al., Emergency Med 22:612-616, 2005). Animal models have demonstrated that naloxone has a minimal effect on vocal cord closure and the upper AW effects of fentanyl in dose ranges relevant to or safe for humans (Willette et al., J Pharmacol Methods 17:15-25, 1987). The mechanism/s of these vocal cord and upper AW effects have not been identified.

Naloxone's effectiveness for reversing fentanyl overdose is possibly limited due to fentanyl's unique potency and binding at non-opiate receptors and/or non-opiate receptor distributions in the brainstem and other regions that control motor efferent output to the chest wall, larynx, vocal cords and respiratory diaphragm. Inappropriate activation of these receptors by fentanyl results in respiratory muscle rigidity and airway paralysis (Fu et al., Anesthesiology. 87(6):1450-1459, 1997; Lui et al., Neurosci Lett. 201(2):167-170, 1995; Milne et al., Can J Physiol Pharmacol. 67(5):532-536, 1989; Lui et al., Neurosci Lett. 108(1-2):183-188, 1990; Lui et al., Neurosci Lett. 96(1):114-119, 1989; Sohn et al., Anesthesiology 103: 327-334, 2005; and Root-Bernstein et al., Int J Mol Sci. 19(1), 2018). Fentanyl has a similar binding affinity (Ki) at mu-opioid receptors as morphine and the leading antagonist drugs used to reverse opioid overdose (e.g. naloxone; Evers, Maze & Kharasch. Anesthetic Pharmacology. Cambridge University Press, 2011; Volpe et al., Regul Toxicol Pharmacol 59(3):385-390, 2011; Clarke et al., Emergency Med 22:612-616, 2005). Given this similar binding affinity of morphine and fentanyl and the fact that naloxone has a greater binding affinity at mu opioid receptors, it is surprising clinically, that fentanyl overdose requires repeated doses of naloxone to reverse its specific effects (Walley et al., Morb Mortal Wkly Rep 66:382-386, 2017; Clarke et al., Emergency Med 22:612-616, 2005; Chou et al., Ann Intern Med 167(12):867-875, 2017). This is a key indicator of fentanyl binding at receptor sites other than the opiate/mu receptors and that FIRMR and/or WCS has a limited relationship to mu receptor activation that has not been fully described to date.

### (III) PROPOSED MODE(S) OF ACTION

The following discussion is provided for context, is based on the knowledge, experience, and professional expertise of the inventor, but in no way is it intended to limit the function or practice of the technology and discoveries described herein. Described herein are proposed pharmacological mechanisms specific to F/FAs and stimulants as previously defined, to facilitate development of more effective treatments for F/FA and stimulant overdose. Prior to this disclosure, there remain several critical issues and/or gaps in the basic knowledge of the underlying mechanisms of F/FA-induced FIRE syndrome, SSOIVE and respiratory depression, including: 1) the false perception that F/FA's effects are similar to morphine-derived opioids, but more potent, and are therefore treatable simply with higher doses of MOR antagonists; 2) current public health data clearly indicate that naloxone is not effective for F/FAs, but there has been little new drug development; 3) previous work with animal models of FIRE syndrome/ FIRMR (e.g., Jerussi et al., Pharmacol Biochem Behav, 28(2):283-289, 1987; Lui et al., Neurosci Lett, 157(2):145-148, 1993; Lui et al., Neurosci Lett, 96(1):114-119, 1989; Lui et al., Neurosci Lett, 108(1-2):183-18, 1990; Lui et al., Neurosci Lett, 201(2):167-170, 1995; Weinger et al., Brain Res, 669(1):10-18, 1995; Yang et al., Anesthesiology, 77(1):153-161, 1992) occurred prior to human studies demonstrating the involvement of vocal cords (VC) in human F/FA induced FIRE syndrome (Bennett et al., Anesthesiology 87(5):1070-1074, 1997). No animal model since has incorporated this VC effect to further explore FIRE syndrome, SSOIVE from F/FA's and prior models bypassed VCs with either endotracheal intubation or tracheostomy, creating a years-long gap in the literature. Therefore, the effects of potential therapies on VC function and upper airway mechanical failure from F/FAs have been unknown. However, the inventor has developed an animal airway model (exemplified in rat), using real time video endoscopy, that demonstrates vocal cord closure and chest wall rigidity after high dose fentanyl (50-100 mcg/kg) within 15-30 seconds after intravenous bolus. These effects persist for ˜90 seconds, whereupon the heart becomes asystolic and arterial pressure falls to 0 (zero) mm Hg and the animal cannot be resuscitated without the administration of therapeutic agents. All respiratory effort ceases at the time onset of vocal cord closure (e.g. 15-30 seconds after IV bolus). This effect is specific to F/FA and is not demonstrated with morphine, heroin, or stimulants. The precise mechanism of action (MOA) whereby fentanyl (carfentanil) increases and/or enhances noradrenaline (NA) outflow from the Locus coeruleus (LC) was still unknown and had not been demonstrated prior to this disclosure , but has been suggested by the data from the series of experiments performed for proof of concept of underlying mechanism and identification of specific molecular targets.

Fentanyl has a significant binding affinity to α-1 B adrenergic receptor subtypes, with a rank binding order of 1B ˜1A and (1:5)>1D (e.g. 1B˜1 A» 1 D) and has been shown to act as an antagonist at these receptor subtypes. Additionally, preliminary data indicates that fentanyl blocks norepinephrine reuptake at the vesicular monoamine transporter- VMAT and thereby enhances the availability of norepinephrine for release from the pre-synaptic terminal. Given that other A1ARAs (e.g. prazosin, tamsulosin) block the effects of norepinephrine (NE) at these receptors and limit NA outflow from the LC, it is difficult to imagine that F/FAs may not have similar effects. However, selective binding by F/FAs at these subtypes can facilitate binding of either norepinephrine- NE and/or epinephrine at the 1D subtype where each of these endogenous neurotransmitters have their greatest binding affinity and sympathomimetic effects. Thus, an antagonist at alpha 1 adrenergic receptors would be expected to limit noradrenergic effects of both F/FAs and stimulants.

Alternative, because fentanyl binds and antagonizes receptor subtypes A-1A and A-1 B, but has a 5 fold less binding affinity for the A-1 D adrenergic receptor subtype, this may allow for unopposed or facilitated agonism, activation, or stimulation of A-1D adrenergic receptors by NE. The NE that is being released in the LC may be caused by fentanyl binding to mu opioid receptors, mu opioid receptors on GABA interneurons, cholinergic receptors and/or some combination of these receptors. Regardless of the MOA, unopposed agonism of isolated α-1 adrenergic receptors (e.g., 1D subtype) could result in profound systemic hypertension from arterial contractility, decreased blood flow/decreased hepatic perfusion and a rapid increase in contractile tone (rigidity) to the muscles of respiration and muscles of the larynx and vocal cords. These effects are further exaggerated when F/FAs are combined with stimulants. It is important to note that these are only some of the possible MOA, most of which have not been suggested or discussed in the literature. Additionally, it is not the intent of this document to be a complete, comprehensive or exhaustive review of all the possible MOAs suggested for FIMR or to be limited in scope by the MOA mentioned here.

Although the MOA of F/FAs is ill-defined and not completely understood, the existing animal data suggests (Fu et aL, Anesthesiology. 87(6):1450-1459, 1997; Lui et aL, Neurosci Lett. 201(2):167-170, 1995; Milne et aL, Can J Physiol Pharmacol. 67(5):532-536, 1989; Lui et aL, Neurosci Lett. 108(1-2):183-188, 1990; Lui et aL, Neurosci Lett. 96(1):114-119, 1989; Sohn et aL, Anesthesiology 103: 327-334, 2005; and Root-Bernstein et aL, Int J Mol Sci. 19(1), 2018) that fentanyl and its analogues (such as sufentanil, alfentanil, remifentanil, and carfentanil) have the ability to bind to (that is, associate specifically with) Mu opioid receptors in the LC of the Pons/brainstem. Through an unclear mechanism that has been only partially explored in previous literature, fentanyl and its analogues cause increased NA flow from the LC and via spinal motor neurons and sympathetic fiber tracts to the muscles of respiration and the intrinsic muscles of the airway, cause fentanyl induced muscle rigidity in animals (FIRMR and/or FIRE syndrome in humans) and life-threatening, mechanical failure of the respiratory system and in some cases the cardiovascular system. However, prior to the animal model described herein using a fiberoptic endoscope to observe vocal cord response to high dose F/FA, the upper airway effect of laryngospasm had not been studied in the animal model even though laryngospasm is the key feature of FIRE syndrome in humans. Conversely and prior to the clinical studies proposed in this application, the neuropharmacologic mechanisms underlying FIRE syndrome have not been studied in humans. However, the upper airway effect of laryngospasm has not been studied in the animal model even though laryngospasm is the key feature of FIRE syndrome in humans. Conversely, the neuropharmacologic mechanisms underlying FIRE syndrome have not been studied in humans. This MOA is difficult to explain because it contradicts the general medical and scientific pharmacologic consensus regarding the action of opiates on the sympathetic nervous system (e.g. opiates/opioid receptor antagonists consistently depress NA neuronal output and sympathetic outflow from the CNS).

This is a mechanism that has been poorly understood and difficult to reconcile with the well-established medical and scientific literature that supports that all opioids, including F/FAs reduce catecholamine levels in the CNS and peripheral nervous systems specifically norepinephrine levels (Aghajanian, The Journal of Clinical Psychiatry 43:20-24, 1982) and the fact that fentanyl acts as an antagonist at all 3 of the alpha 1 adrenergic subtypes (Sohn et al., Anesthesiology 103:327-334, 2005) similar to other alpha 1 adrenergic antagonists (prazosin and terazosin) yet the reaction of F/FA induced FIRMR and/or FIRE syndrome in humans does not occur without noradrenergic activation of the LC and conversely is completely inhibited by the administration of high dose alpha 1 adrenergic antagonist agents. The doses of prazosin used are in a high dose range that would be lethal to humans, making the information unusable, therefore a better more detailed elucidation and understanding of the mechanism will be required to design safe and effective therapy for the FIRMR and/or FIRE syndrome in humans and in this case, to treat SSOIVE effects from combining F/FAs and stimulants. Another significant limitation in the previous work/studies is that the effects of this therapy on VCC laryngospasm was not studied or evaluated. It has been suggested that naloxone (mu antagonism) is not effective for preventing VCC in this model (Willette et al., J Pharmacol Methods 17:15-25, 1987) and unclear whether alpha adrenergic antagonism would be effective since the sole innervation of the laryngeal muscles is controlled by the parasympathetically dominant vagus nerve. Vagal motor neurons are more likely to involve cholinergic innervation based on the parasympathetic tone via vagal nerve fibers to the laryngeal muscles which controls all intrinsic muscles of the larynx. The most effective treatment for laryngospasm may involve the modulation of cholinergic motor neurons with muscarinic receptors (M1-M5), although this has not been demonstrated in the animal model. The fact that fentanyl may act as an antagonist at M3 receptors may also facilitate selective binding of Ach at the M1 M2 M4 receptors and facilitate activity of the laryngeal muscles.

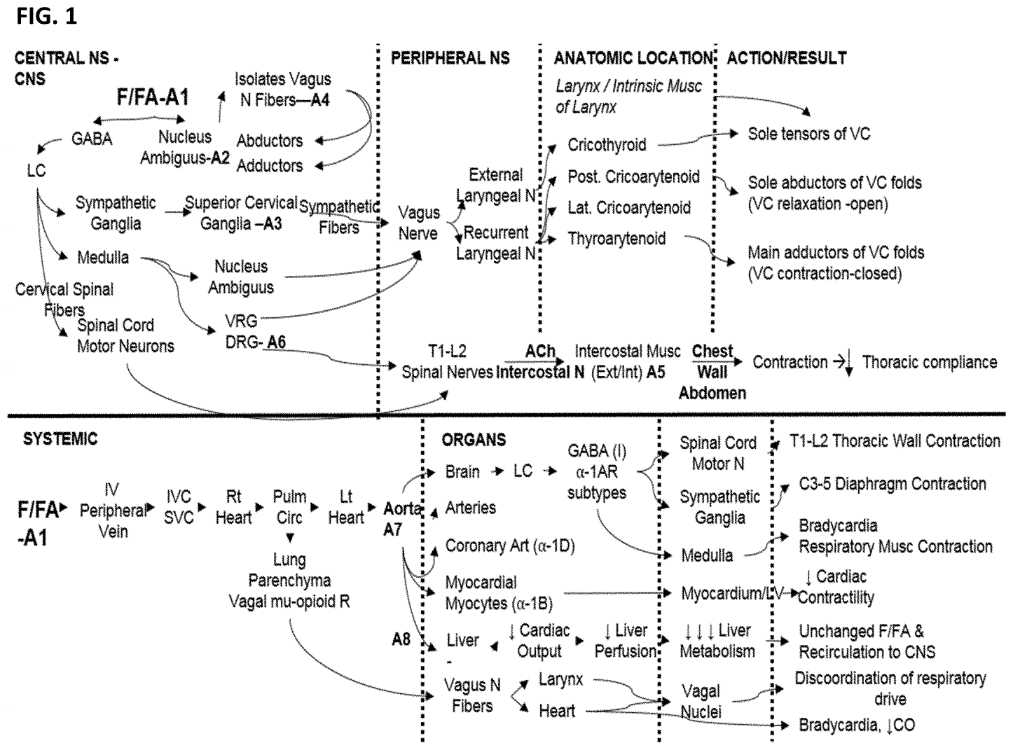

The LC is a key component or target in the treatment of , FIRMR and/or FIRE syndrome in humans and to treat SSOIVE effects from combining F/FAs and stimulants, because it has the highest concentration of noradrenergic neurons in the entire mammalian CNS, is the major production site of noradrenaline in the CNS, and the key nexus communicating with medullary and pontine respiratory nuclei controlling afferent and efferent motor control to the muscles of respiration including the larynx and vocal cords. It has neural fibers that run to and provide noradrenergic input to nearly all major structures in the brain including the cortex, thalamus, amygdala, and the raphe nucleus and to the centers in the brainstem such as the medulla, spinal motor neurons, and to the ventral and dorsal horns (VH, DH) of the spinal cord (FIG. 1). The DH and VH are primarily and densely populated with NA neurons and a adrenergic receptors. Stimulation of these a adrenergic receptors with NA either experimentally or in vivo results in excitation and contraction of terminal sites on skeletal muscle fibers located in the chest wall, abdominal wall and diaphragm (Fu et al., Anesthesiology. 87(6):1450-1459, 1997; Lui et al., Neurosci Lett. 201(2):167-170, 1995; Milne et al., Can J Physiol Pharmacol. 67(5):532-536, 1989; Lui et al., Neurosci Lett. 108(1-2):183-188, 1990; Lui et al., Neurosci Lett. 96(1):114-119, 1989; Sohn et al., Anesthesiology 103: 327-334, 2005; and Root-Bernstein et al., Int J Mol Sci. 19(1), 2018). In the case of the sympathetic neurons, this takes the dermatomal and spinal nerve distribution of vertebral levels T1-L2, which maps to the thoracic /chest wall, abdominal muscles, and part of the diaphragm in humans (FIG. 1). If the chest wall muscles get contracted in a large volume or maximal inspiration via the external intercostal muscles, this can trigger afferent signals from “stretch” or “J” receptors in the lung parenchyma and chest wall that then go back to the Dorsal Respiratory Group (DRG) and Ventral Respiratory Group (VRG) groups of neurons located in the major respiratory center in the medulla region of the brainstem via the vagal nerves. This is known as the “Hering-Breur reflex” arc. Activation of the DRG and VRG or activation via these reflex arc results in increased excitability of the efferent motor neurons with the end result being skeletal muscle contraction in the external intercostal muscles of the chest wall, abdominal wall and diaphragm and increased contractility of the larynx and closure of vocal cords (FIG. 1). Similarly, increased NA outflow from the LC can travel to sympathetic innervation of the vocal cords via superior cervical ganglia and the vagal fibers innervating the laryngeal muscles, mediating adductor activation and/or abductor relaxation resulting in laryngospasm. In the case of the cardiovascular system, the vascular tone and heart function is predominantly controlled by alpha adrenoceptors and the availability of norepinephrine and epinephrine to agonize them. This takes on greater significance in the setting of stimulant and F/FA polysubstance overdose and treating FIRE syndrome and SSOIVE effects from combining F/FAs and stimulants.

Regarding naloxone in high doses in this setting of F/FA ad stimulant overdose may exacerbate noradrenergic and/or catecholamine release and may worsen hypertension, arrhythmias, tachycardia, seizures, pulmonary hypertension, pulmonary edema and myocardial depression. Treatment and resuscitation may require the combination of vasoactive drugs that can control these catecholamine and sympathetic effects that can be worsened from high dose naloxone in the setting of F/FA ad stimulant overdose. It is likely the case that naloxone will be ineffective in reversing respiratory defects due to the fact that the majority of respiratory and vascular effects in F/FA stimulant overdose will be sympathetically driven via alpha and beta adrenergic receptors and naloxone will worsen symptoms due it increasing catecholamine release.

As an Anesthesiologist in clinical practice for more than 20 years, I have administered fentanyl and fentanyl analogues to more than 20,000 patients, amounting to several hundred thousand doses. I have clinically treated FIRMR and/or FIRE syndrome in humans on a number of occasions, and my years of clinical experience and knowledge from having seen and treated this phenomenon first hand have provided me with a unique perspective and clinical insight into the underlying molecular mechanism of FIRMR and/or FIRE syndrome in humans that resulted in the discoveries described herein.

In addition to my clinical observations in Anesthesia, I have worked and trained extensively as an Addictionologist and have been able to further consolidate and confirm my knowledge of FIRMR and/or FIRE syndrome and SSOIVE effects from combining F/FAs and stimulants from eyewitness accounts and interviews with survivors of fentanyl overdose or witnesses to F/FA overdose deaths. From these accounts, I was able to correlate my clinical observations and treatment of FIRMR and/or FIRE syndrome as an anesthesiologist with the clinical presentations of F/FA and stimulant overdose and would conclude that the underlying mechanism of death in F/FA is actually FIRMR and/or FIRE syndrome and SSOIVE effects from combining F/FAs and stimulants. The consistency of the clinical presentations described combined with my clinical experience with FIRMR and/or FIRE syndrome and vasoactive agents to manipulate blood pressure and hemodynamics in surgery, have given me the knowledge and skill to develop treatments for SSOIVE effects from combining F/FAs and stimulants as described here and teaching their implementation to the public.

One blinded case study arose from a public discussion I had with an individual who was not a patient of mine (and with whom I have no personal or professional relationship). Despite that individual having limited to no medical knowledge or knowledge of wooden chest syndrome, they provided the detail of an overdose with a sub-lethal dose/known quantity of fentanyl and effectively described in what I can only call “textbook detail”, the engagement of the external intercostal muscles in a maximal inspiratory position and acute vocal cord (VC) closure that was persistent for approximately three minutes before a loss of consciousness (LOC) occurred.

A single study (Sohn et al., Anesthesiology 103:327-243, 2005) showed that fentanyl could bind to the A-1 B, A and 1D adrenergic receptors as an antagonist in isolated segments of canine pulmonary artery with such affinity that it could competitively block the potent effects of the al B agonist, phenylephrine (e.g., phenylephrine has similar binding capacity to Noradrenaline at the alpha-1B receptor subtype) at concentrations [microM/10⁻⁶M] which are thought to be within the range of the therapeutic serum/tissue levels and concentrations of fentanyl (e.g. 10-25 ng/ml approximates a 10⁻⁷M (Yamanoue et al., Anesthesia & analgesia 76:382-390, 1993) and 2.96x10⁻³M for brain lipid (Stone & DiFazio, Anesthesia & analgesia 67:663-666, 1988; Sohn et al., Anesthesiology 103:327-334, 2005) concentration in the CNS) that are also found on autopsy from deaths caused by fentanyl and FAs. However, the binding affinity values were not clearly described in the study itself and cannot be compared directly to norepinephrine binding affinity values, as there are no clear values available from current scientific literature. In a series of experiments designed by the inventor, the values of norepinephrine at these receptors was determined and compared to binding by F/FAs and used to elucidate the underlying binding pattern and mechanism for FIRE syndrome and SSOIVE effects from combining F/FAs and stimulants (See figures and Tables 1-3). This data was then used to design the formulations described.

In turn, each of the α-1 adrenergic antagonists has a unique binding distribution at the α-1 subtypes. For example, the selective agent Tamsulosin has a 12-30-times greater binding affinity at the 1 A subtype over other α-1 antagonists and greater binding affinity than Prazosin. Tamsulosin has similar potency at the 1D subtype. As a result of its subtype specificity, Tamsulosin has a lower impact on blood pressure compared to the non-selective agents such as Prazosin. Both agents have the ability to cross the blood brain barrier and thus can bind to α-1 receptors in the pons and LC. Thus, one embodiment provides a strategy to mitigate effects on hemodynamics/blood pressure by combining both agents (at a selected ratio, such as 1:1, 2:1 ,3:1 in favor of the α1A selective agent) to allow for a decrease in hypotensive side effects (e.g. “first dose effect”) while optimizing antagonism of α-1 subtypes with each agent. In the case of SSOIVE, we may want to optimize these effects to decrease the severe hypertensive effects seen from combining F/FAs and stimulants. In this case, Tamsulosin binds 1A and 1D subtypes while Prazosin is able to bind 1B adrenergic receptors at a dose that is lower than if prazosin were used as a single agent. This strategy allows for optimal antagonism of FIRE syndrome and SSOIVE effects while limiting the side effect profile of the non-selective agent Prazosin. This strategy is discussed further below.

Although the medical literature has described vocal cord-(VC) spasm/laryngospasm with FIRMR, the underlying molecular mechanisms have not been described in humans and the available animal data makes no observations of the effect of F/FA on the upper airway (e.g. larynx, vocal cords). This lack of data specific to the underlying mechanism has precluded development of the precise pharmacologic interventions necessary to prevent overdose from F/FA and F/FA combined with stimulants. The inventor's direct clinical observation that spasm of the VC was not immediately relieved by the muscle paralytic-succinylcholine, which acts in the periphery of skeletal muscle acetylcholine receptors (AchRs) suggests that F/FAs effects on the larynx and vocal cords is a centrally-mediated effect that may come from the LC, pontine(pons) and medullary(medulla) circuitry, as described above. The pathway for VC spasm/laryngospasm may come from several mechanisms such as direct activation of motor efferents in the medulla (e.g. VRG neurons, nucleus ambiguus) by way of NA neurons from the pons/LC or directly at cholinergic receptors in medullary nuclei by F/FAs themselves. Some studies also suggest that NA activation in the pons/LC may be mediated via increased ACH release into the LC by surrounding cholinergic nuclei and serves to increase NE release in the LC. As described herein, subtype specific binding of NE to al AR causes specific changes in respiratory mechanics that can lead to respiratory failure and death. These effects are further accelerated by sympathetic drugs mediation of increased NE and also impact the cardiovascular system.

The literature prior to the priority date of this application adds no clear explanation or complete picture of the mechanism of action of fentanyl or FAs in FIRE syndrome and SSOIVE effects or FIRMR, particularly at the level of the alpha-1 adrenergic subtypes or at cholinergic receptors and the MOA remains unclear and non-obvious. Additionally, a significant limitation of the prior literature is that doses used in prior animal experiments were not meant to induce or increase human survival rates from FIRE syndrome and SSOIVE effects in F/FA and stimulant overdose, but simply to demonstrate a possible MOA for FIMR and no set of animal experiments has demonstrated the effects of combining these 2 drug classes. None of the animals survived those experiments. As such, the doses of al -adrenergic antagonists used in those animal experiments would be routinely fatal to a substantial portion of subjects given such doses. Thus, the previously available animal data could not have been used to develop therapeutics without significant experimentation and further understanding of the underlying molecular mechanisms and the development of an animal model with clinical validity to airway effects seen in humans and as elucidated and taught for the first time herein. Thus, the previously available animal data could not be used to develop therapeutics without significant modification, substantial discovery and subsequent discovery knowledge, as taught for the first time herein.

The results in prior animal experiments were obtained through the use of dosing strategies that would cause significant mortality and morbidity in human subjects and have not considered the SSOIVE effects in F/FA and stimulant overdose, thus, dosing levels presented in the animal data are unfeasible in humans without a significant modification in the side effect profile and use of an appropriate animal model that combines F/FAs and stimulants in simulated overdose or the use of molecules that demonstrate significant synergy and/or a clearer understanding of the underlying mechanism of FIRE syndrome and SSOIVE effects in F/FA and stimulant overdose in humans. In addition, other cholinergically mediated mechanisms of laryngospasm in FIRE syndrome remained unexplored in animals and humans, that is until the pre-clinical development of this invention. Dosing strategies using al agents that are appropriate to human subjects have not been explored until now, with the provision herein of combinations for therapeutic compounds to treat WCS/ FIRMR in F/FA overdose or toxic exposure.

A goal here is to use either synergy between molecules, to alleviate side-effects and/or to improve/diminish the side effect profile of prazosin (e.g. severe orthostatic hypotension, syncope, life-threatening or severe hypotension, myocardial ischemia) to make treatment of FIRE syndrome and SSOIVE effects in F/FA and stimulant overdose feasible in humans and is the key to being able to use this technology to improve the survival rate of F/FAs and stimulant overdose.

Example treatments and methods described herein take advantage of and/or utilize the unique α-1 adrenergic receptor subtype binding affinities of different α-1 adrenergic antagonists, so as to optimize α-1 subtype antagonism while minimizing α-1 antagonist side effects (Including the primarily life-threatening hypotension that occurs with the non-selective agents). This is more of an advantage issue for prophylaxis agents that prevent life-threatening hypertensive crises when the individual is exposed to SSOIVE effects in F/FA and stimulant overdose, but minimizes or eliminates episodes of severe hypotension (low blood pressure) prior to exposure to F/FAs and stimulants. A combination of selective and non-selective α-1 antagonist agents is an exemplary dosing strategy to maximize receptor antagonism while minimizing mortality and morbidity from severe vascular and hemodynamic instability or compromise.

Thus, provided herein are dosing strategies using combinations of α-1 adrenergic receptor antagonist(s) and one or more other supportive agent(s) to minimize side effects and optimize survival and outcomes from of FIRE syndrome and SSOIVE effects in F/FA and stimulant overdose. Additionally, a beta blocker (e.g. Atenolol, esmolol, propranolol), may be optionally added to antagonize the potential direct or indirect effects of fentanyl and F/FAs and/or stimulants on alpha and beta adrenergic receptors.

### (IV) THERAPEUTIC COMPOUNDS

Provided herein are pharmaceutical compositions, as well as methods of their use. Generally, these compositions include one or more of a therapeutically effective amount of a1-adrenergic receptor antagonist, in some embodiments in combination with a therapeutically effective amount of one or more of a Mu or opioid receptor subtype antagonist and/or a cholinergic agent (muscarinic antagonist/ M3 agonist and/or nicotinic agonist) and/or a centrally-acting or peripherally acting respiratory stimulant and /or a GABA/benzodiazepine receptor complex antagonist, and in certain embodiments a Mu or opioid receptor subtype agonist, long-acting Mu or opioid receptor subtype antagonist, vasoactive agents for blood pressure support, anticholinergic agents, a centrally-acting a adrenergic receptor antagonist combined with a peripherally acting a adrenergic receptor antagonist, muscle paralytic and anticonvulsant or membrane-stabilizing agents. Additionally, a beta blocker (e.g. Atenolol, esmolol, propranolol), may be optionally added to antagonize the potential direct or indirect effects of fentanyl and F/FAs and/or stimulants on alpha and beta adrenergic receptors.

The overall treatment goal of these combined agents is minimization of FIRE syndrome and SSOIVE effects in F/FA and stimulant overdose. Optionally, the composition also includes a pharmaceutically acceptable carrier, such as lipophilic agents or nano-particle technology or other carriers discussed herein and/or known in the art for delivery as IV, IM, INH, IO, PO etc. For instance, eye drops (IOC delivery) is a simple method of drug administered that can be used to effectively deliver agents in to the CNS, as the eye is an extension of the CNS itself. 10C may represent a particularly beneficial route of delivery to the CNS, given that pilocarpine (M3 agonist) and atropine are and can readily be administered as eyedrops in the case of anticholinergic or cholinergic treatment. Similarly, inhaled (INH) delivery can be used, for instance for prophylaxis, in a nebulizer, metered-dose inhaler (MDI), or as a vaping or vaporization INH solution. Reversal compositions can be delivered via INH routes, if the airway is patent or delivery made via endotracheal tube.

Mu or opioid receptor subtype antagonists are used herein for alleviating or inhibiting the dose dependent respiratory depression caused by all opiates/opioids and any intermediary effects leading to activation or antagonism of other receptor subtypes (e.g. GABA interneurons, alpha adrenergic receptors, cholinergic receptors) and the vasoactive agents (e.g. alpha 1 antagonists, alpha 2 agonists, beta blockers) are for treating FIRE syndrome and SSOIVE effects in F/FA and stimulant overdose. Short duration and rapid acting agents (e.g., naloxone, NARCAN®, nalmefene) are used for immediate reversal, while longer acting agents (e.g., naltrexone, nalmefene) can be used for prophylaxis.

Alpha adrenergic receptor antagonists (AARAs) are used herein to inhibit of FIRE syndrome and SSOIVE effects in F/FA and stimulant overdose. In various embodiments, selective or non-selective antagonists or combination agents (e.g. alpha adrenergic antagonist and anticholinergic antagonist, such as droperidol) are used either singly or in combination to minimize the effects of AARAs on blood pressure as may be needed with prophylaxis agents and will use delivery into the CNS via nasal insufflation to minimize the peripheral effects of AARAs on blood pressure. In addition, AARAs will be used in combination with vasoactive agents as noted above to offset, counteract or minimize the effects of the unfavorable effects of AARAs on blood pressure and hemodynamics. Additionally a beta blocker (e.g. Atenolol, esmolol, propranolol), may be optionally added to antagonize the potential direct or indirect effects of fentanyl and F/FAs and/or stimulants on alpha and beta adrenergic receptors such as severe hypertension or tachycardia. This is more of an advantage issue for prophylaxis agents that prevent life-threatening hypertensive crises when the individual is exposed to SSOIVE effects in F/FA and stimulant overdose, but minimizes or eliminates episodes of severe hypotension (low blood pressure) and or hypertension (high blood pressure) prior to exposure to F/FAs and stimulants. However, these hypotensive effects are clearly advantageous and particularly helpful at times of overdose resuscitation since most patients will be hemodynamically hyperstimulated from increased sympathomimetic effects of combining F/FAs with stimulants and in those cases a non-selective agent (e.g. prazosin) for immediate reversal may be of greater advantage. Additionally. a beta blocker (e.g. Atenolol, esmolol, propranolol), may be optionally added to antagonize the potential direct or indirect effects of fentanyl and F/FAs and/or stimulants on alpha and beta adrenergic receptors. These combinations can be used in either immediate reversal agents or in prophylaxis compounds

Anticholinergic agents can be used herein, in patients who are either bradycardic or asystolic, to decrease vagal tone (baseline heart rate) or to alleviate cholinergically mediated closure of vocal cords/laryngospasm in patients who are using these drugs for prophylaxis or immediate reversal, but should be avoided in patients who are tachycardic and or demonstrating ventricular or tachycardic arrhythmias.

Muscle relaxants and paralytics (such as succinylcholine and rocuronium) are rapid acting, and optionally can be used as described herein to alleviate and overdose related to F/FAs particularly in chest wall and diaphragm and may help to relieve spasm of the vocal cords and larynx or if these symptoms are enhanced by the combination of F/FAs and stimulants. Although low doses on the order 1-3 mg for Succinylcholine can be used to decrease or to inhibit of FIRE syndrome and overdose related to F/FAs, it is preferable to use full intubation doses (e.g. 1-1.5 mg/kg) to secure the airway with an endotracheal tube. These drugs would be used in immediate resuscitation scenarios by individuals who are trained in invasive AW management.

Similarly, an anticonvulsant such as Dilantin can be added to this compound to act as prophylaxis against seizures that can occur with FIRE syndrome and SSOIVE effects in F/FA and stimulant overdose.

It is contemplated that the therapeutic agents can be administered to a subject (for instance, a subject in need of prevention or reversal of one or more effect of an opiate or opioid compound taken concurrently with a stimulant) at the same time, or in sequence/series, in various embodiments and with various durations of onset and action as described herein. In embodiments that contain two (or more) different therapeutic compounds (that is, combination formulations or combined therapeutics), optimally the pharmaceutical composition includes a set proportion or proportion range of one therapeutic compound to another in the composition. Some examples would include, a combined therapeutic in some embodiments with a ratio of 0.5-1 parts naloxone to 1 parts prazosin; and/or similar combinations with longer acting mu opioid receptor antagonists and selective or non-selective alpha 1 antagonists. These exemplary ratios are on the higher side of the dosing range and can be scaled lower and are not meant to be a complete or limiting description here of all the ratios that can be effectively utilized. Additional description of compounds useful for the compositions and methods described herein are discussed below.

The disclosure provides a platform of compounds and molecules that either singly or in combination block/antagonize/modulate or prophylax against the effects of piperidine derived opioids (e.g. fentanyl and fentanyl analogues) combined with a stimulant on the neurophysiology and mechanics of respiration, with the addition of one or more other molecules to either synergize reversal of F/FAs overdose or offset side effects of dose requirements required for optimal treatment. The platform also includes the use of F/FAs in combination with an A1ARA to optimize analgesia with prophylaxis against WCS/ FIRMR.

The following are descriptions of representative compounds that are applicable to be used in one or more of the therapeutic combination treatments provided herein. Many of these compounds have recognized, well-known safety profiles and dosing strategy guidelines, though guidance is provided herein. VIVITROL® (naltrexone for extended-release injectable suspension) and Nasal NARCAN® (naloxone hydrochloride) are listed below as examples of industry acceptable delivery methods for intramuscular (IM) extended-release injectables and formula solutions for nasal insufflation, respectively. Dosing charts provided herein supply an abbreviated summary of dosages and practitioner guidelines for the use of representative product(s) / compound(s) as is suitable for the clinical presentation requiring treatment.

(a) α1-Adrenergic Receptor Antagonists

α-1 adrenergic receptor blockers inhibit vasoconstriction by blocking norepinephrine binding to α-1 post synaptic membrane receptors, which inhibits the blood vessels from contraction and can block norepinephrine effects centrally in the LC. It happens because α 1 blockers inhibit the activation of post-synaptic α-1 receptors and prevent the release of catecholamines (Sica, J Clin Hyperten. 7(12):757-762, 2005). α-1 adrenergic receptor antagonists block a receptors and relax the smooth muscles in the vascular system and bladder. Alpha-1 blockers lower blood pressure by blocking α-1 receptors so norepinephrine can't bind the receptor causing arterial vessels to dilate. In view of these vascular effects, selective α-1 blockers are better tolerated than non-selective a blockers, due to less hypotension. Terazosin, tamsulosin and doxazosin are prime drugs prophylaxis because they have a long half-life and modified release formulations and have selectivity for alpha 1D receptor subtypes. Tamsulosin is particularly ideal because it minimally affects the blood pressure and the side effects of vasodilation is minimal compared to less selective agents (prazosin) (Kaplan, Am J Med. 80(56):100-104, 1986). See also Yoshizumi et al. (Am J Physiol Renal Physiol 299: F785-F791, 2010, showing binding of tamsulosin to the LC in Pons).

This class of molecules is of key importance in the formulation of compounds and pharmacologic treatment for WCS/ FIRMR, due to their direct antagonistic effects on α1 adrenergic receptors located on noradrenergic neurons in the central nervous system (e.g. cortex, thalamus, brainstem, spinal cord) and vascular and muscle tissue (e.g. smooth and skeletal) in the periphery.

α-1 adrenergic receptor antagonists (AARAs) are used to inhibit FIMR in animal models, but have not been demonstrated to be effective in humans or animals for F/FA induced WCS/FIRMR. In various embodiments, selective or non-selective antagonists are used either singly or in combination to minimize the effects of AARA on blood pressure and will use delivery into the CNS via nasal insufflation to minimize the peripheral effects of AARAs on blood pressure. In addition, in certain embodiments AARAs are used in combination with vasoactive and cholinergic agents to offset, counteract, or minimize the effects of the unfavorable effects of AARAs on blood pressure and hemodynamics. This may be particularly helpful at times of overdose resuscitation, at which time most patients will be hemodynamically depressed. These combinations can be used in either immediate reversal or in prophylaxis embodiments.

TAMSULOSIN: Dose (0.4-0.8 mg QD); incidence of hypotension, syncope, vertigo is 0.2%-0.6% (˜1 in 500). Tamsulosin hydrochloride is a selective antagonist of α1A adrenoceptors in the prostate. Tamsulosin hydrochloride is (-)−(R)-5-[2-[[2-(o-Ethoxyphenoxy) ethyl]amino]propyl]-2-methoxybenzenesulfon-amide, monohydrochloride. Tamsulosin hydrochloride is a white crystalline powder that melts with decomposition at approximately 230° C. It is sparingly soluble in water and methanol, slightly soluble in glacial acetic acid and ethanol, and practically insoluble in ether.

The empirical formula of tamsulosin hydrochloride is C₂₀H₂₈N₂O₅S·HCl. The molecular weight of tamsulosin hydrochloride is 444.98. Its structural formula is:

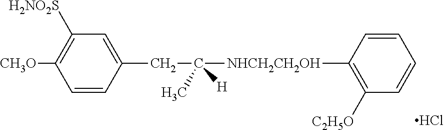

PRAZOSIN: Dose is 1 mg BID/TID and can be titrated up to 20 mg total QD in divided doses 5-6 mg TID). Syncope and symptoms of hypotension are 6-12% of subjects receiving (˜90 in 900).

MINIPRESS® (prazosin hydrochloride), a quinazoline derivative, is the first of a new chemical class of antihypertensives. It is the hydrochloride salt of 1-(4-amino-6,7-dimethoxy-2-quinazolinyl)-4-(2-furoyl) piperazine and its structural formula is:

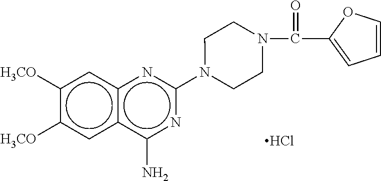

It is a white, crystalline substance, slightly soluble in water and isotonic saline, and has a molecular weight of 419.87. Each 1 mg capsule of MINIPRESS for oral use contains drug equivalent to 1 mg freebase. Molecular formula C₁₉H₂₁N₅O₄·HCI.

TERAZOSIN (dose 1-5 mg QD and NTE 20 mg QD) causes significant hypotension like prazosin with postural hypotension levels of 4% in trial of 600 subjects. syncope was 0.6%. HYTRIN (terazosin hydrochloride), an α-1-selective adrenoceptor blocking agent, is a quinazoline derivative represented by the following chemical name and structural formula: (RS)-Piperazine,1-(4-amino-6,7-dimethoxy-2-quinazolinyl)-4-[(tetra-hydro-2-furanyl)carbonyl]-, monohydrochloride, dihydrate.

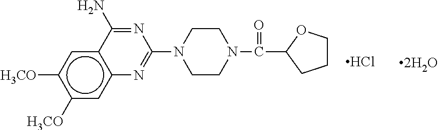

Terazosin hydrochloride is a white, crystalline substance, freely soluble in water and isotonic saline and has a molecular weight of 459.93. HYTRIN tablets (terazosin hydrochloride tablets) for oral ingestion are supplied in four dosage strengths containing terazosin hydrochloride equivalent to 1 mg, 2 mg, 5 mg, or 10 mg of terazosin.

SILODOSIN: (Dose: 8 mg QD) Study of 897 subjects with 3% with Dizziness and orthostatic hypotension and 1 /897 with syncope.

RAPAFLO is the brand name for silodosin, a selective antagonist of α-1 adrenoreceptors. (3-Hydroxypropyl)-5-[(2R)-2-({2[2-(2,2,2trifluoroethoxy)phenoxy]ethyl}amino)propyl]-2,3-dihydro-1H-indole-7-carboxamide and the molecular formula is C₂₅H₃₂F₃N₃O₄ with a molecular weight of 495.53. The structural formula of silodosin is:

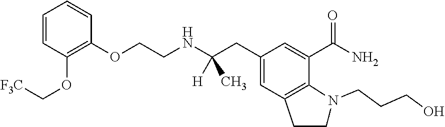 [SMILES: C[C@H](Cc1cc2c(c(C(N)=O)c1)N(CCCO)CC2)NCCOc1ccccc1OCC(F)(F)F]

Silodosin is a white to pale yellowish white powder that melts at approximately 105 to 109° C. It is very soluble in acetic acid, freely soluble in alcohol, and very slightly soluble in water.

ALFUZOSIN: (Dose: 10-15 mg) 473 test subjects 6% had dizziness, 1/473 0.2% with syncope and 2/473 0.4% with hypotension. UROXATRAL® (alfuzosin HCl) Extended-release Tablets

Each UROXATRAL extended-release tablet contains 10 mg alfuzosin hydrochloride as the active ingredient. Alfuzosin hydrochloride is a white to off-white crystalline powder that melts at approximately 240° C. It is freely soluble in water, sparingly soluble in alcohol, and practically insoluble in dichloromethane. Alfuzosin hydrochloride is (R,S)—N-[3-[(4-amino-6,7-dimethoxy-2-quinazolinyl) methylamino] propyl] tetrahydro-2-furancarboxamide hydrochloride. The empirical formula of alfuzosin hydrochloride is C₁₉H₂₇N₅O₄·HCl. The molecular weight of alfuzosin hydrochloride is 425.9. Its structural formula is:

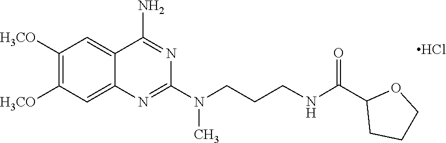 [SMILES: COc1cc2nc(N(C)CCCNC(=O)C3CCCO3)nc(N)c2cc1OC ; Cl]

DOXAZOSIN: (dose : 1 mg QD NTE 16 mg, dose may be titrated up to 2 mg q 1-2 weeks ; 1-16 mg in HTN and 0.5-8 mg in normotensives) 965 test subjects Dizzy 15-19% and Hypotension in 1.7%.

CARDURA® (doxazosin mesylate) CARDURA® (doxazosin mesylate) is a quinazoline compound that is a selective inhibitor of the α1 subtype of α-adrenergic receptors. The chemical name of doxazosin mesylate is 1-(4-amino-6,7-dimethoxy-2-quinazolinyl)-4-(1,4benzodioxan-2-ylcarbonyl) piperazine methanesulfonate. The empirical formula for doxazosin mesylate is C₂₃H₂₅N₅O₅·CH₄O₃S and the molecular weight is 547.6. It has the following structure:

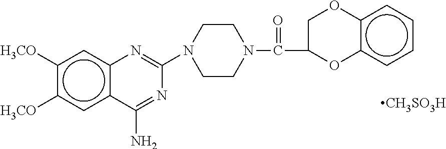

CARDURA (doxazosin mesylate) is freely soluble in dimethylsulfoxide, soluble in dimethylformamide, slightly soluble in methanol, ethanol, and water (0.8% at 25° C.), and very slightly soluble in acetone and methylene chloride. CARDURA is available as colored tablets for oral use and contains 1 mg (white), 2 mg (yellow), 4 mg (orange) and 8 mg (green) of doxazosin as the free base.

Beta blockers (beta-blockers, β-blockers, etc.) are a class of medications that are predominantly used to manage abnormal heart rhythms, and to protect the heart from a second heart attack (myocardial infarction) after a first heart attack (secondary prevention).They are also widely used to treat high blood pressure (hypertension) and tachycardia. Beta blockers are competitive antagonists that block the receptor sites for the endogenous catecholamines epinephrine (adrenaline) and norepinephrine (noradrenaline) on adrenergic beta receptors, of sympathetic nervous system. Some block activation of all types of β-adrenergic receptors and others are selective for one of the three known types of beta receptors, designated β₁, β₂ and β₃ receptors. β₁-adrenergic receptors are located mainly in the heart and in the kidneys. β₂-adrenergic receptors are located mainly in the lungs, gastrointestinal tract, liver, uterus, vascular smooth muscle, and skeletal muscle. Beta receptors are found on cells of the heart muscles, smooth muscles, airways, arteries, kidneys.

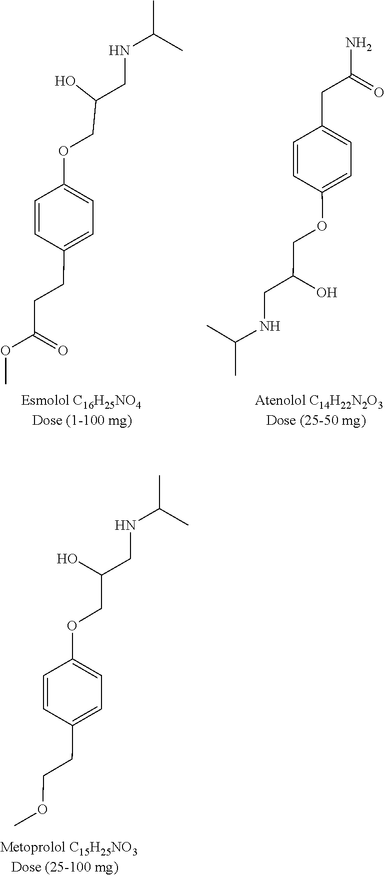 [SMILES: COC(=O)CCc1ccc(OCC(O)CNC(C)C)cc1 ; CC(C)NCC(O)COc1ccc(CC(N)=O)cc1 ; COCCc1ccc(OCC(O)CNC(C)C)cc1]

(b) Mu and/or opioid receptor subtype antagonists

Mu receptor antagonists are used for alleviating or inhibiting the dose dependent respiratory depression caused all opiates/opioids and can vary in their effects at opioid receptor subtypes (delta, kappa, mu). Short duration and rapid acting agents (e.g. naloxone, NARCAN®) are used for immediate reversal, while longer acting agents (e.g. naltrexone) are used for prophylaxis. MU receptor antagonists include Naloxone, Naltrexone, Nalmefene, nalorphine, and Levallorphan.

NALOXONE—NARCAN® (dose 0.4-2 mg IV and may repeat dose up to 10 mg. May also be dosed IM, SC, intranasal) (naloxone hydrochloride) NARCAN (naloxone hydrochloride injection, USP), an opioid antagonist, is a synthetic congener of oxymorphone. In structure it differs from oxymorphone in that the methyl group on the nitrogen atom is replaced by an allyl group; the structure is provided below.

Naloxone hydrochloride occurs as a white to slightly off-white powder, and is soluble in water, in dilute acids, and in strong alkali; slightly soluble in alcohol; practically insoluble in ether and in chloroform. NARCAN (naloxone) injection is available as a sterile solution for intravenous, intramuscular and subcutaneous administration in three concentrations: 0.02 mg, 0.4 mg and 1 mg of naloxone hydrochloride per mL. pH is adjusted to 3.5±0.5 with hydrochloric acid. The 0.02 mg/mL strength is an unpreserved, paraben-free formulation containing 9 mg/mL sodium chloride.

NARCAN (naloxone) may be diluted for intravenous infusion in normal saline or 5% dextrose solutions. Naloxone is indicated for the complete or partial reversal of opioid depression, including respiratory depression, induced by natural and synthetic opioids. NARCAN (naloxone) is also indicated for diagnosis of suspected or known acute opioid overdosage. If an opioid overdose- is known or suspected: an adult initial dose of 0.4 mg to 2 mg of NARCAN (naloxone) may be administered intravenously, IM, subcutaneously or nasally. If the desired degree of counteraction and improvement in respiratory functions are not obtained, it may be repeated at two- to three-minute intervals. If no response is observed after 10 mg of NARCAN (naloxone) have been administered, the diagnosis of opioid-induced or partial opioid-induced toxicity should be questioned. If necessary, NARCAN (naloxone) can be diluted with sterile water for injection.

NALOXONE NASAL SPRAY FORMULATION: NARCAN® (naloxone hydrochloride) Nasal Spray. NARCAN (naloxone hydrochloride) Nasal Spray is a pre-filled, single dose intranasal spray. Chemically, naloxone hydrochloride is the hydrochloride salt of 17-Allyl-4,5a-epoxy-3,14- dihydroxymorphinan-6-one hydrochloride with the following structure:

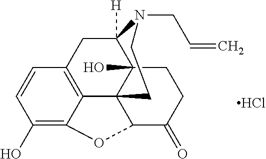

Naloxone hydrochloride, an opioid antagonist, occurs as a white to slightly off-white powder, and is soluble in water, in dilute acids, and in strong alkali; slightly soluble in alcohol; practically insoluble in ether and in chloroform. Each NARCAN Nasal Spray contains a single 4 mg dose of naloxone hydrochloride in a 0.1 MI intranasal spray. Inactive ingredients include benzalkonium chloride (preservative), disodium ethylenediaminetetraacetate (stabilizer), sodium chloride, hydrochloric acid to adjust pH, and purified water. The pH range is 3.5 to 5.5. NARCAN Nasal Spray is indicated for the emergency treatment of known or suspected opioid overdose, as manifested by respiratory and/or central nervous system depression. NARCAN Nasal Spray is intended for immediate administration as emergency therapy in settings where opioids may be present.

NALTREXONE: REVIA® (DOSE 25-50 MG PO QD) (naltrexone hydrochloride) Tablets USP 50 mg -long acting opioid antagonist. REVIA® (naltrexone hydrochloride tablets USP), an opioid antagonist, is a synthetic congener of oxymorphone with no opioid agonist properties. Naltrexone differs in structure from oxymorphone in that the methyl group on the nitrogen atom is replaced by a cyclopropylmethyl group. REVIA is also related to the potent opioid antagonist, naloxone, or n-allylnoroxymorphone.

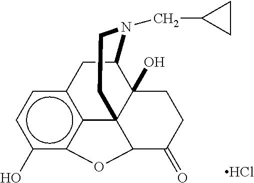

REVIA is a white, crystalline compound. The hydrochloride salt is soluble in water to the extent of about 100 mg/mL. REVIA is available in scored film-coated tablets containing 50 mg of naltrexone hydrochloride. REVIA Tablets also contain: colloidal silicon dioxide, crospovidone, hydroxypropyl methylcellulose, lactose monohydrate, magnesium stearate, microcrystalline cellulose, polyethylene glycol, polysorbate 80, synthetic red iron oxide, synthetic yellow iron oxide and titanium dioxide.

VIVITROL®-NALTREXONE INJECTABLE: Extended-release Injectable Suspension: VIVITROL® (naltrexone for extended-release injectable suspension) is supplied as a microsphere formulation of naltrexone for suspension, to be administered by intramuscular injection. Naltrexone is an opioid antagonist with little, if any, opioid agonist activity. Naltrexone is designated chemically as morphinan-6-one, 17 (cyclopropylmethyl) 4,5-epoxy 3,14-dihydroxy-(5α) (CAS Registry # 16590-41-3). The molecular formula is C₂₀H₂₃NO₄ and its molecular weight is 341.41 in the anhydrous form (i.e., <1% maximum water content). The structural formula is:

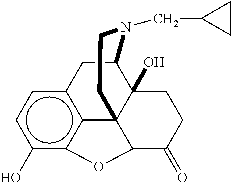

Naltrexone base anhydrous is an off-white to a light tan powder with a melting point of 168-170° C. (334-338° F.). It is insoluble in water and is soluble in ethanol. VIVITROL is commercially available as a carton containing a vial each of VIVITROL microspheres and diluent, one 5-mL syringe, one 1-inch 20-gauge preparation needle, two 1° -inch 20-gauge and two 2-inch 20-gauge administration needles with needle protection device. VIVITROL microspheres consist of a sterile, off-white to light tan powder that is available in a dosage strength of 380 mg of naltrexone per vial. Naltrexone is incorporated in 75:25 polylactide-co-glycolide (PLG) at a concentration of 337 mg of naltrexone per gram of microspheres. The diluent is a clear, colorless solution. The composition of the diluent includes carboxymethylcellulose sodium salt, polysorbate 20, sodium chloride, and water for injection. The microspheres must be suspended in the diluent prior to injection.

NALMEFENE: REVEX (nalmefene hydrochloride) Injection, Solution.

REVEX (nalmefene hydrochloride injection), an opioid antagonist, is a 6-methylene analogue of naltrexone. The chemical structure is shown below:

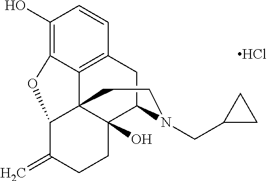 [SMILES: C=C1CC[C@@]2(O)[C@H]3Cc4ccc(O)c5c4[C@@]2(CCN3CC2CC2)[C@H]1O5 ; Cl]

Molecular Formula: C₂₁H₂₅NO₃·HCl; Molecular Weight: 375.9, CAS # 58895-64-0; Chemical Name: 17-(Cyclopropylmethyl)-4,5a-epoxy-6-methylenemorphinan-3,14-diol, hydrochloride salt.

Nalmefene hydrochloride is a white to off-white crystalline powder which is freely soluble in water up to 130 mg/mL and slightly soluble in chloroform up to 0.13 mg/mL, with a pKa of 7.6.

REVEX is available as a sterile solution for intravenous, intramuscular, and subcutaneous administration in two concentrations, containing 100 μg or 1.0 mg of nalmefene free base per mL. The 100 pg/mL concentration contains 110.8 μg of nalmefene hydrochloride and the 1.0 mg/mL concentration contains 1.108 mg of nalmefene hydrochloride per mL. Both concentrations contain 9.0 mg of sodium chloride per mL and the pH is adjusted to 3.9 with hydrochloric acid. Concentrations and dosages of REVEX are expressed as the free base equivalent of nalmefene.

REVEX is indicated for the complete or partial reversal of opioid drug effects, including respiratory depression, induced by either natural or synthetic opioids. REVEX is indicated in the management of known or suspected opioid overdose. REVEX should be titrated to reverse the undesired effects of opioids. Once adequate reversal has been established, additional administration is not required and may actually be harmful due to unwanted reversal of analgesia or precipitated withdrawal.

(c) Paralytics/Muscle Relaxants

Muscle relaxants and paralytics such as succinylcholine and rocuronium are rapid acting and are used to alleviate WCS /FIRMR particularly in the chest wall and diaphragm, and may relieve spasm of the vocal cords and larynx. Low doses (on the order 1-3 mg for Succinylcholine) can be used to decrease FIMR without compromise of AW reflexes. These drugs are generally used in immediate resuscitation scenarios by individuals who are trained in invasive AW management and are used at full intubation doses (0.5-1.1 mg/kg).

Succinylcholine: (Anectine, QUELICIN™) For reversal or inhibition of fentanyl or fentanyl analogue induced muscle rigidity- FIMR in an adult patient, dose is 0.01-0.05 mg/kg. However, to fully secure the airway with endotracheal intubation, the dose is 0.3-1.1 mg/kg. QUELICINTM (succinylcholine chloride) Injection, USP

QUELICIN (Succinylcholine Chloride Injection, USP) is a sterile, nonpyrogenic solution to be used as a short-acting, depolarizing, skeletal muscle relaxant. The solutions are for I.M. or I.V. use. Succinylcholine Chloride, USP is chemically designated C₁₄H₃₀Cl₂N₂O and its molecular weight is 361.31. It has the following structural formula:

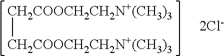

Succinylcholine is a diquaternary base consisting of the dichloride salt of the dicholine ester of succinic acid. It is a white, odorless, slightly bitter powder, very soluble in water. The drug is incompatible with alkaline solutions but relatively stable in acid solutions. Solutions of the drug lose potency unless refrigerated. Solution intended for multiple-dose administration contains 0.18% methylparaben and 0.02% propylparaben as preservatives (List No. 6629). Solution intended for single-dose administration contains no preservatives. May contain sodium hydroxide and/or hydrochloric acid for pH adjustment. pH is 3.6 (3.0 to 4.5). Succinylcholine chloride is indicated as an adjunct to general anesthesia, to facilitate tracheal intubation, and to provide skeletal muscle relaxation during surgery or mechanical ventilation and for the treatment of fentanyl induced chest wall or muscle rigidity (Janssen Pharmaceuticals package insert for “Sublimaze-Fentanyl”).

The dosage of succinylcholine should be individualized and should always be determined by the clinician after careful assessment of the patient. For Reversal or inhibition of fentanyl or fentanyl analogue induced rigidity in an adult patient, dose is 0.01 — 0.05 mg/kg. However, for full scale securing of the airway with endotracheal intubation in severe WCS, the dose is 0.3-1.1 mg/kg. Following administration of doses in this range, neuromuscular blockade develops in about 1 minute; maximum blockade may persist for about 2 minutes, after which recovery takes place within 4 to 6 minutes. However, very large doses may result in more prolonged blockade. A 5 to 10 mg test dose may be used to determine the sensitivity of the patient and the individual recovery time.

Whereas bradycardia is common in pediatric patients after an initial dose of 1.5 mg/kg, bradycardia is seen in adults only after repeated exposure. The occurrence of bradyarrhythmias may be reduced by pretreatment with atropine.

If necessary, succinylcholine may be given intramuscularly to adults when a suitable vein is inaccessible. A dose of up to 3 to 4 mg/kg may be given, but not more than 150 mg total dose should be administered by this route. The onset of effect of succinylcholine given intramuscularly is usually observed in about 2 to 3 minutes.

Succinylcholine is acidic (pH 3.5) and should not be mixed with alkaline solutions having a pH greater than 8.5 (e.g., barbiturate solutions). QUELICINTM (Succinylcholine Chloride Injection, USP) is supplied as a clear, colorless solution. Refrigeration of the undiluted agent will assure full potency until expiration date. All units carry a date of expiration. Store in refrigerator 2° to 8° C. (36° to 46° F.). The multi-dose vials are stable for up to 14 days at room temperature without significant loss of potency.

ROCURONIUM: ZEMURON® (rocuronium bromide) Injection (dose: for intubation 0.4-1.2 mg/kg and for treatment of FIMR 0.005-0.01 mg/kg). ZEMURON (rocuronium bromide) injection is a nondepolarizing neuromuscular blocking agent with a rapid to intermediate onset depending on dose and intermediate duration. Rocuronium bromide is chemically designated as 1[17β-(acetyloxy)-3α-hydroxy-2β-(4-morpholinyl)-5α-androstan-16β-yl]-1-(2-propenyl)pyrrolidinium bromide. The structural formula is:

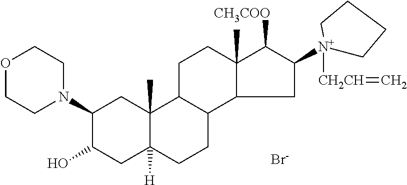

The chemical formula is C₃₂H₅₃BrN₂O₄ with a molecular weight of 609.70. The partition coefficient of rocuronium bromide in n-octanol/water is 0.5 at 20° C. ZEMURON is supplied as a sterile, nonpyrogenic, isotonic solution that is clear, colorless to yellow/orange, for intravenous injection only. Each mL contains 10 mg rocuronium bromide and 2 mg sodium acetate. The aqueous solution is adjusted to isotonicity with sodium chloride and to a pH of 4 with acetic acid and/or sodium hydroxide. ZEMURON® (rocuronium bromide) Injection is indicated for inpatients and outpatients as an adjunct to general anesthesia to facilitate both rapid sequence and routine tracheal intubation, and to provide skeletal muscle relaxation during surgery or mechanical ventilation and for the treatment of fentanyl induced muscle rigidity—FIMR or WCS.

ZEMURON is for intravenous use only. This drug should only be administered by experienced clinicians or trained individuals supervised by an experienced clinician familiar with the use, actions, characteristics, and complications of neuromuscular blocking agents. Doses of ZEMURON injection should be individualized and a peripheral nerve stimulator should be used to monitor drug effect, need for additional doses, adequacy of spontaneous recovery or antagonism, and to decrease the complications of overdosage if additional doses are administered. The dosage information which follows is derived from studies based upon units of drug per unit of body weight. It is intended to serve as an initial guide to clinicians familiar with other neuromuscular blocking agents to acquire experience with ZEMURON. The recommended initial dose of ZEMURON, regardless of anesthetic technique, is 0.6 mg/kg. Neuromuscular block sufficient for intubation (80% block or greater) is attained in a median (range) time of 1 (0.4-6) minute(s) and most patients have intubation completed within 2 minutes. Maximum blockade is achieved in most patients in less than 3 minutes. In appropriately premedicated and adequately anesthetized patients, ZEMURON 0.6 to 1.2 mg/kg will provide excellent or good intubating conditions in most patients in less than 2 minutes.

(d) α2-Adrenergic Receptor Agonists

In certain embodiments, alpha 2 agonists may be used in the inhibition or partial inhibition of fentanyl induced muscle rigidity. Optionally, these can be used with an α1 antagonist in various treatment methods. Clonidine is a representative α2-adrenergic receptor agonist.

Clonidine-CATAPRES® (clonidine hydrochloride) Oral Antihypertensive Tabs of 0.1, 0.2 and 0.3 mg, CATAPRES® (clonidine hydrochloride, USP) is a commercially available centrally acting alpha-agonist hypotensive agent available as tablets for oral administration in three dosage strengths: 0.1 mg, 0.2 mg and 0.3 mg. The 0.1 mg tablet is equivalent to 0.087 mg of the free base. The inactive ingredients are colloidal silicon dioxide, corn starch, dibasic calcium phosphate, FD&C Yellow No. 6, gelatin, glycerin, lactose, and magnesium stearate. Clonidine hydrochloride is an imidazoline derivative and exists as a mesomeric compound. The chemical name is 2-(2,6-dichlorophenylamino)-2-imidazoline hydrochloride; C₉H₉Cl₂N₃HCl, Mol. Wt. 266.56. Clonidine hydrochloride is an odorless, bitter, white, crystalline substance soluble in water and alcohol. The following is the structural formula:

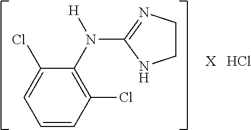

The following is a general guide to its administration. Initial dose: 0.1 mg tablet twice daily (morning and bedtime). Elderly patients may benefit from a lower initial dose. Maintenance Dose: Further increments of 0.1 mg per day may be made at weekly intervals if necessary until the desired response is achieved. Taking the larger portion of the oral daily dose at bedtime may minimize transient adjustment effects of dry mouth and drowsiness. The therapeutic doses most commonly employed have ranged from 0.2 mg to 0.6 mg per day given in divided doses. Studies have indicated that 2.4 mg is the maximum effective daily dose, but doses as high as this have rarely been employed. In the case of F/FA overdose or toxic exposure 0.05 mg-1 mg will be diluted into sterile water or NS for IV or IM injection in combination with other agents as noted in dosing charts.

(e) GABA/benzodiazepine receptor complex antagonists

Dilantin and Flumazenil are given in a ratio of 50 mg/0.2 mg as a prophylaxis against the risk or occurrence of seizures due to rapid benzodiazepine reversal in drug overdoses involving individuals with regular or habitual use of benzodiazepines. In the event of “status epilepticus” induced by rapid reversal of benzodiazepine overdose, a conversion to use of separate baseline reversal drug (e.g. MU+NS-A1ARA+S-A1ARA) with IV Dilantin (5-15 mg/kg) with infusion rate NTE 50 mg/min due to risk of cardiac arrhythmia.

Romazicon (flumazenil) Injection, USP: GCA: FLUMAZENIL 0.2 MG may repeat Q 2-3″ 0.2-1 mg total administered IV, IN. Flumazenil Injection, USP is a benzodiazepine receptor antagonist. Chemically, flumazenil is ethyl 8-fluoro-5,6-dihydro-5-methyl-6-oxo-4H-imidazo[1,5-a](1,4) benzodiazepine-3-carboxylate. Flumazenil has an imidazobenzodiazepine structure, a calculated molecular weight of 303.3, and the following structural formula:

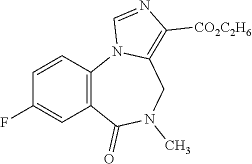

Flumazenil is a white to off-white crystalline compound with an octanol: buffer partition coefficient of 14 to 1 at pH 7.4. It is insoluble in water but slightly soluble in acidic aqueous solutions. Flumazenil injection is available as a sterile parenteral dosage form for intravenous administration. Each mL contains 0.1 mg of flumazenil compounded with 1.8 mg of methylparaben, 0.2 mg of propylparaben, 0.9% sodium chloride, 0.01% edetate disodium, and 0.01% acetic acid; the pH is adjusted to approximately 4 with hydrochloric acid and/or, if necessary, sodium hydroxide.

For the reversal of the sedative effects of benzodiazepines administered for conscious sedation, the recommended initial adult dose of flumazenil injection is 0.2 mg (2 mL) administered intravenously over 15 seconds. If the desired level of consciousness is not obtained after waiting an additional 45 seconds, a second dose of 0.2 mg (2 mL) can be injected and repeated at 60-second intervals where necessary (up to a maximum of 4 additional times) to a maximum total dose of 1 mg (10 mL). The dosage should be individualized based on the patient's response, with most patients responding to doses of 0.6 mg to 1 mg. In the event of re-sedation, repeated doses may be administered at 20-minute intervals as needed. For repeat treatment, no more than 1 mg (given as 0.2 mg/min) should be administered at any one time, and no more than 3 mg should be given in any one hour. It is recommended that flumazenil injection be administered as the series of small injections described (not as a single bolus injection) to allow the practitioner to control the reversal of sedation to the approximate endpoint desired and to minimize the possibility of adverse effects.

### (V) COMPOSITIONS FOR METHODS OF USE

The compounds disclosed herein can be formulated into compositions for direct administration to a subject for prophylaxis against or reversal of F/FA induced to inhibit of FIRE syndrome and SSOIVE effects in F/FA and stimulant overdose and the vascular effects of stimulants that may be enhanced by noradrenergic activities of either drug or the combination of stimulants with fentanyl/fentanyl analogues. Increased noradrenergic activity in the case of each drug will enhance the catastrophic effects of fentanyl/fentanyl analogues manifested as FIRE syndrome and SSOIVE effects in F/FA and stimulant overdose and the vascular effects of stimulants that may be enhanced by noradrenergic activities of either drug or the combination of stimulants with fentanyl/fentanyl analogues and severe cardiovascular and cerebrovascular effects seen with stimulant overdose. The combination of these drugs enhances the noradrenergically driven side effects of each drug. It is contemplated that the compounds may be administered to the same subject in concert, whether sequentially or simultaneously. The significant point regarding administration is that naloxone as a single agent, is ineffective and/or minimally effective in reversing the symptoms of FIRE syndrome in humans and the airway effects and vascular effects of stimulants that may be enhanced by noradrenergic activities of either drug or the combination of stimulants with fentanyl/fentanyl analogues, must be combined with other agents as noted in these compositions to be effective.

Specific combinations of compounds (Formula Equations) for use in several embodiments provided herein include the following [where MU =Mu receptor and/or opioid receptor subtype antagonists, MUXR=Extended release Mu receptor and/or opioid receptor subtype antagonists A1ARA=Alpha-1 Adrenergic receptor antagonist, A2ARA=Alpha-2 Adrenergic receptor agonist, BetaB=Beta Blocker, PMR=Paralytic/Muscle relaxant, S=selective, NS=non-selective , GCA=GABA Complex Antagonist, and ASMS=Anti-seizure/Membrane stabilizer]:

Representative IMMEDIATE REVERSAL NON-MEDICAL (IRNM) embodiments:

(IRNM1) MU+S-A1ARA

(IRNM2) MU+A2ARA

(IRNM3) MU+NS-A1ARA

(IRNM4) MU+S-A1ARA+/−NS-A1ARA

(IRNM5) MU+S-A1ARA+/−NS-A1ARA+/−A2ARA

(IRNM6) MU+S-A1ARA+/−NS-A1ARA+/−AC or C

(IRNM7) MU+S-A1ARA+/−NS-A1ARA+/−AC or C+/−A2ARA or +/−BetaB

Representative IMMEDIATE REVERSAL MEDICAL NO AW (IRMnAW) embodiments: (including all of the previous embodiments in addition can be administered as an alternative to these formulations):

(IRMnAW1) MU+S-A1ARA+/−NS-A1ARA

(IRMnAW2) MU+S-A1ARA+/−NS-A1ARA+/−AC or C

(IRMnAW3) MU+S-A1ARA+NS-A1ARA+A2ARA or +/−BetaB

Representative IMMEDIATE REVERSAL MEDICAL AW (IRMAW) embodiments (including all of the above previous embodiments in addition can be administered as an alternative to these formulations):

(IRMAW1) MU+S-A1ARA+/−NS-A1ARA

(IRMAW2) MU+S-A1ARA+/−NS-A1ARA+/−PMR or +/−BetaB

Representative POLYSUBSTANCE (Poly) embodiments:

(Polyl) MU+S-A1ARA+NS-A1ARA+GCA

(Poly2) MU+S-A1ARA+NS-A1ARA+GCA +ASMS

(Poly3) MU+S-A1ARA+NS-A1ARA+GCA +ASMS+PMR or +/−BetaB

Representative PROPHYLAXIS for ACTIVE STIMULANT and Synthetic OPIOID USER (PASOU) embodiment:

(PASOU1) MU (naltrexone or nalmefene)+S-A1ARA+/−NS-A1ARA; or

(PASOU2) MU+S-A1ARA+NS-A1ARA+A2ARA or +/−BetaB

PROPHYLAXIS for FIRST RESPONDER FOR STIMULANT and Synthetic OPIOID (PRF) embodiment

(PFR1) MU or MUXR+S-A1ARA+/−NS-A1ARA or +/−BetaB

Specific example dosage delivery systems are as follows: Intranasal (IN), sterile normal saline nasal solution (e.g., same % concentration and composition as standard 0.9% NaCI solution and pH adjusted to accommodate optimal solubility and deliverability of the molecules contained as solutes for delivery into the CNS); Intraocular (IOC), sterile normal saline or suitable ocular solution (e.g., % concentration, composition and pH adjusted to accommodate optimal solubility and deliverability of the molecules contained as solutes for delivery into the CNS); Intravenous (IV), sterile normal saline intravenous solution (e.g. same % concentration and composition as standard 0.9% NaCI solution); Intrathecal (IT), sterile isobaric, hypobaric and hyperbaric dextrose solutions for Intrathecal-CNS injection; Transdermal (TD), sterile slow release lipid matrix for transdermal absorption; intramuscular injection (IM), sterile slow release lipid matrix for intramuscular injection-IM and steady—state absorption; Intraosseous (IO), sterile normal saline intravenous solution (e.g. same % concentration and composition as standard 0.9% NaCI solution); sublingual formulation (e.g. rapid dissolving tablet or strip) Oral formulation (e.g. capsule, tablet or gel cap); transtracheal atomization-sterile normal saline intravenous solution (e.g. same % concentration and composition as standard 0.9% NaCI solution); nebulizer - sterile normal saline intravenous solution (e.g. same % concentration and composition as standard 0.9% NaCI solution); and Metered dose inhaler (MDI). Thus, in various embodiments administration is via oral, sublingual-SL, intravenous-IV, intramuscular-IM, transdermal-TD, nasal insufflation-NI, inhalation-MDI, intraosseous injection-IO, intrathecal-IT injection, transtracheal-TT injection or atomization or intraocular-IO.

In particular embodiments, the therapeutic compounds are provided as part of composition that can include at least 0.1% w/v or w/w of therapeutic compounds; at least 1% w/v or w/w of therapeutic compounds; at least 10% w/v or w/w of therapeutic compounds; at least 20% w/v or w/w of therapeutic compounds; at least 30% w/v or w/w of therapeutic compounds; at least 40% w/v or w/w of therapeutic compounds; at least 50% w/v or w/w of therapeutic compounds; at least 60% w/v or w/w of therapeutic compounds; at least 70% w/v or w/w of therapeutic compounds; at least 80% w/v or w/w of therapeutic compounds; at least 90% w/v or w/w of therapeutic compounds; at least 95% w/v or w/w of therapeutic compounds; or at least 99% w/v or w/w of therapeutic compounds.

The compositions disclosed herein can be formulated for administration by, injection, inhalation, infusion, perfusion, lavage, topical ocular delivery or ingestion. The compositions disclosed herein can further be formulated for infusion via catheter, intravenous, intramuscular, intratumoral, intradermal, intraarterial, intranodal, intralymphatic, intraperitoneal, topical, intrathecal, intravesicular, oral and/or subcutaneous administration and more particularly by intravenous, intradermal, intraarterial, intranodal, intralymphatic, intraperitoneal, topical, intrathecal, intratumoral, intramuscular, intravesicular, oral and/or subcutaneous injection.

For injection and infusion, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions, and the like. For oral solid formulations such as, for example, powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For administration by inhalation, compositions can be formulated as aerosol sprays from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the therapeutic and a suitable powder base such as lactose or starch.

Any composition formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts.

Additionally, compositions can be formulated as sustained-release systems utilizing semipermeable matrices of solid polymers containing at least one active ingredient. Various sustained-release materials have been established and are well known by those of ordinary skill in the art. Sustained-release systems may, depending on their chemical nature, release active ingredients following administration for two weeks to 1 month. In particular embodiments, a sustained-release system could be utilized, for example, if a human patient were to miss a weekly administration.

Specific expected formulations include those intended for immediate delivery, for instance where at least one (or each) component of the therapeutic system is provided in an immediate acting drug delivery system (for instance, IV, IO, CNS-Intrathecal injection, INH-metered dose inhaler, or Nasal spray administration). In other specific embodiments, the formulations include those intended for intermediate delivery, in which at least one (or each) component of the therapeutic system is provided in an intermediate acting delivery system. (for instance, oral extended release, or IM administration). In such intermediate delivery embodiments, onset generally in less than 1 hour, and duration is generally for up to 48 hours. Yet further embodiments provide extended release systems, for instance, extended release systems for prophylaxis. In such extended release systems, at least one (or each) component of the therapeutic system is provided in a long acting delivery system (for instance, slow release oral, extended release IM administration, or gel matrix patch). Onset for such extended release systems is generally within one hour or more, with resultant duration up to 60 days.

### (VI) METHODS OF USE

Methods disclosed herein include treating subjects (including humans, veterinary animals, livestock, and research animals) with compositions disclosed herein. As indicated, the compositions can treat a variety of different conditions, including intentional or accidental exposure to and/or overdose with one or more opiate or opioid compounds, or a mixture containing at least one opiate or opioid compound; or one or more symptoms associated with opiate/opioid overdose (including but not limited to FIRMR, laryngospasm and/or FIRE syndrome) or symptoms associated with stimulant overdose (cardiovascular effects such as myocardial infarction or arrhythmia, and/or cerebrovascular effects such as stroke or hypertensive crisis) and/or combined with fentanyl or a fentanyl analogue. Specific examples of methods of use, including clinical settings in which such use might occur, are provided in Table 1 and the text associated therewith, as well as the Examples.

Treating subjects includes delivering therapeutically effective amounts of one or more composition(s). Therapeutically effective amounts can provide effective amounts, prophylactic treatments, and/or therapeutic treatments.

An “effective amount” is the amount of a compound necessary to result in a desired physiological change or effect in the subject. Effective amounts disclosed herein result in partial or complete reversal or prevention of a symptom of opiate/opioid exposure or overdose following administration to a subject.

A “prophylactic treatment” includes a treatment administered to a subject who does not display signs or symptoms of a condition or displays only early signs or symptoms of the condition such that treatment is administered for the purpose of diminishing, preventing, or decreasing the risk of developing the condition further or in anticipation of exposure to the toxin or offensive chemical agent. Thus, a prophylactic treatment functions as a preventative treatment.

A “therapeutic treatment” includes a treatment administered to a subject who displays symptoms or signs of a condition and is administered to the subject for the purpose of diminishing or eliminating one or more of those signs or symptoms of the condition.

Prophylactic and therapeutic treatments need not fully prevent or cure a condition but can also provide a partial benefit.

One embodiment of the method involves use of a Mu opioid receptor and/or opioid receptor subtype (mu, kappa, delta receptor subtypes) antagonist (e.g. naloxone, naltrexone, nalmefene) in combination with an Alpha-adrenergic receptor antagonist-AARA (e.g. prazosin, terazosin, tamsulosin, doxazosin) and/or a cholinergic agent (muscarinic receptor antagonist/ anticholinergic, M3 receptor agonist or a nicotinic receptor general or selective agonist) for immediate reversal of FIRMR, laryngospasm and/or FIRE syndrome and overdose related to F/FAs or F/FAs combined with morphine or morphine derivatives or cardiovascular or cerebrovascular events and/or SSOIVE effects related to stimulant overdose or combined stimulant and fentanyl/fentanyl analogue overdose. Additionally a beta blocker (e.g. Atenolol, esmolol, propranolol), may be optionally added to antagonize the potential direct or indirect effects of fentanyl and F/FAs and/or stimulants on alpha and beta adrenergic receptors.

Another embodiment of the method involves use of a mu opioid receptor and/or opioid receptor subtype (mu, kappa, delta receptor subtypes) antagonist in combination with an a-adrenergic receptor antagonist-AARA and a rapid acting muscle paralytic (e.g. succinylcholine, rocuronium) to synergistically interact with AARA to reduce or reverse FIRMR, laryngospasm and/or FIRE syndrome and for immediate reversal with a clinical presentation of severe or persistent respiratory muscle rigidity and/ or laryngospasm. Additionally a beta blocker (e.g. Atenolol, esmolol, propranolol), may be optionally added to antagonize the potential direct or indirect effects of fentanyl and F/FAs and/or stimulants on alpha and beta adrenergic receptors.

Another embodiment of the method involves use of an extended-release mu opioid receptor and/or opioid receptor subtype (mu, kappa, delta receptor subtypes) antagonist (e.g. naltrexone, nalmefene) in combination with an a-adrenergic receptor antagonist for prophylaxis against FIRE syndrome in a population at risk for environmental exposure or overdose due to F/FAs or symptoms associated with stimulant overdose (cardiovascular effects such as myocardial infarction or arrhythmia, and/or cerebrovascular effects such as stroke or hypertensive crisis) when combined with fentanyl or a fentanyl analogue. Additionally a beta blocker (e.g. Atenolol, esmolol, propranolol), may be optionally added to antagonize the potential direct or indirect effects of fentanyl and F/FAs and/or stimulants on alpha and beta adrenergic receptors.

### (VI) KITS

Combinations of active components (including specifically synergistic combinations) can be provided as kits. Kits can include containers including one or more or more compounds as described herein, optionally along with one or more agents for use in combination therapy. For instance, some kits will include an amount of at least one a-adrenergic receptor antagonist (for instance, a centrally acting or peripherally acting a-adrenergic receptor antagonist, or a combination thereof), along with an amount of at least one Mu opioid receptor antagonist and/or another opioid receptor subtype (mu, kappa, delta receptor subtypes) antagonist (for instance, a long-acting Mu receptor antagonist), an a2-adrenergic receptor agonist, a Mu receptor agonist, vasoactive agents (e.g. Vasodilators), anticholinergic agents and/or cholinergic agents (muscarinic receptor antagonist/ anticholinergic, M3 receptor agonist or a nicotinic receptor general or selective agonist). The overall treatment goal of these combined agents is minimize of FIRE syndrome and SSOIVE effects in F/FA and stimulant overdose and the vascular effects of stimulants that may be enhanced by noradrenergic activities of either drug or the combination of stimulants with fentanyl/fentanyl analogues.

Specific contemplated kits included kits tailored to the user of the kit, for instance, an untrained provider kit, a medically trained provider kit (which for instance, may include a vital sign algorithm dosing chart), an emergency administration kit, and so forth. Table 1 provides information regarding types of compounds (and representative compounds) that would be included in certain different kit types.

Similarly, different kits may be provided for different routes of delivery, including for IV, IM, IN, IO, IT, IOC, and TT delivery.

Any active component in a kit may be provided in premeasured dosages, though this is not required; and it is anticipated that certain kits will include more than one dose, including for instance when the kit is used for a method requiring administration of more than one dose of the synergistic combination.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding preparation of component(s) of the synergistic combination, for administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made. The instructions of the kit will direct use of the active ingredients to effectuate a clinical use described herein. In effect, this document offers instruction in the formulation of compounds and the administration of these compounds for the treatment of (prophylaxis or reversal) WCS and other respiratory and muscular effects of F/FAs and morphine derived alkaloids.

### (VIII) EXEMPLARY EMBODIMENTS

The following examples are provided to illustrate certain particular features and/or embodiments. These examples should not be construed to limit the disclosure to the particular features or embodiments described.

The herein provided technology has two general modes of use:
1. Immediate opioid reversal treatment for someone who has overdosed on F/FAs, opioids, or a combination of morphine derived opiates combined with F/FAs and a stimulant. Optionally, the immediate reversal composition also includes drug(s) that may antagonize the benzodiazepine class and is categorized as “polysubstance” reversal.
2. Prophylaxis treatment for someone who is likely to have exposure to F/FAs and/or stimulants, for instance by environmental exposure, or by intentional/ unintentional use of IV opioids or over-ingestion of opioids containing fentanyl or fentanyl analogues or F/FAs combined with a stimulant and/or a morphine derived opiate.

In each general mode of use, the use of compounds (and the corresponding preparations to be used in such methods) is further subdivided according to the (known or expected) baseline skill set of the “provider” or “responder” as either being “non-medical”, “medical provider without AW training or”, “medical provider with AW training.” The assignment of possible compounds that can be used by each type of provider is made according to skill set and clinical presentation for the discernment of the medically trained providers. Examples 2-6 below provide description of how such different responders might use composition(s) provided herein in a method of reversing or preventing one or more effects of opioid/opiate dosing, largely in the format that may be used for packaging instructions or other product-associated literature.

### (IX) EXAMPLES

### Example 1

### Production of Baseline Formulation Doses

This example describes representative dosage amounts of compounds for use in combination therapies described herein. Lower doses can be employed, but improvement of clinical outcome is less likely to be affected or effective at lower doses. Similarly, higher doses can be used, but can negatively impact the overall clinical outcome and survival rates. The baseline formulation doses are designed so that the initial dose can be elevated proportionally by administering additional doses until FIRMR and/or FIRE syndrome or overdose condition is reversed or stabilized and/or cardiovascular or cerebrovascular events related to stimulant overdose (e.g., SSOIVE) or combined stimulant and fentanyl/fentanyl analogue overdose are reversed or stabilized and/or cardiovascular or cerebrovascular events related to stimulant overdose or combined stimulant and fentanyl/fentanyl analogue overdose are reversed or stabilized.

In many situations, 1-4 doses will be sufficient for treatment, but the number and size of dose can be modified to accommodate severe or persistent symptoms from overdose. The chart below for BASE DOSE COMPOUND (BDC) is a guide and is not meant to be limited to dose examples, route and ranges listed below.

TABLE 2
BASE DOSE COMPOUND (BDC), assuming 70 kg adult (±10 kg):

| | Representative | BDC-Ideal | | | Dose Range/ |
|---|---|---|---|---|---|
| Class | Compound(s) | Dose | | Timing | Route |
| BETA B | ESMOLOL | 5-10 | MG | May repeat Q 2-3 | 5-100 MG |
| MU | NALOXONE | 1 MG | | May repeat Q 2-3″ | 0.2-4 mg total |
| | | (14 mcg/kg) | | | IV, IM, IN, IO, IOC |
| | NALTREXONE | 25 | MG | QD | 25-50 mg total |
| | | | | | IV, IM, PO, IN, IO, IOC |
| | NALMEFENE | 0.4-1 MG | | May repeat Q 2-3″ | NTE 5 mg total |
| | | (20-40 mcg/kg) | | | IV, IM, IN, IO, IOC |
| A1ARA | NS - PRAZOSIN | 0.25-0.5 MG* | | May repeat Q 2-3″ | 0.1-20 mg total |
| | | (3-7 mcg/kg) | | | IV, IM, IN, IO, IOC |
| | S- TAMSULOSIN | 0.2-0.4 MG | | May repeat Q 2-3″ | 0.1-0.8 mg total |
| | | 3-6 mcg/kg) | | | IV, IM, IN, IO, IOC |
| | TERAZOSIN | 1 | MG | May repeat Q 2-3″ | 0.5-5 MG NTE 5 MG total |
| | | | | | IV, IM, IN, IO, IOC |
| PMR | SUCCINYLCHOLINE | 1-3 MG | | May repeat Q 2-3″ | 0.5-1.1 mg/kg |
| | | (14-28 mcg/kg) | | | IV, IM, IN, IO |
| GCA | FLUMAZENIL | 0.2 | MG ** | May repeat Q 2-3″ | 0.2 mg-1 mg |
| | | | | | IV, IM, IN, IO, IOC |
| ASMS | DILANTIN | 50 | MG*** | May repeat Q 2-3″ | NTE 50 mg/min rapid |
| | | | | | IV infusion |
| | | | | | IV, IN, IO, IOC |

With regard to table 2, above:
*All of these drug, with the exception of the A1ARAs prazosin and tamsulosin (see “A1ARA IV/IN/IM formulation protocol”) are currently available as IV formulations and therefore can be easily converted to nasal dosing regimens, which are similar in potency and concentration, if not the same, and will be concentratable in a nasal, IV or IM formulation. Both prazosin and tamsulosin can be solubilized and made suitable for IV injection or IN insufflation by standard compounding pharmaceutical techniques.
** Dilantin and Flumazenil will be given in a ratio of 50 mg/0.2 mg as a prophylaxis against the risk or occurrence or seizures due to rapid benzodiazepine reversal in drug overdoses involving individuals with regular or habitual use of benzodiazepines.
***In the event of “status epilepticus” induced by rapid reversal of benzodiazepine overdose, a conversion to use of separate baseline reversal drug (e.g. MU + NS-A1ARA + S-A1ARA) with IV Dilantin (5-15 mg/kg) with infusion rate NTE 50 mg/min due to risk of cardiac arrhythmia.

Epinephrine is to be used with caution in individuals with F/FAs and stimulant overdose due to the direct and potent activity of Epinephrine and Noradrenaline at the LC and FIRE syndrome and SSOIVE related circuitry or cardiovascular or cerebrovascular events related to stimulant overdose or combined stimulant and fentanyl/fentanyl analogue overdose or Stimulant and Synthetic Opioid Induced Vascular Events (SSOIVE) from opioid and stimulant overdose. However, should this be the initial presentation in “Suspected Opioid Overdose”, the medical practitioner should use their discretion to follow best practices and go directly to the most current ACLS treatment algorithms with the possible addition of the “Baseline formulation” for FIMR reversal. The ACLS dose protocol for cardiac arrest — 1 mg IV and may repeat Q2-3″ for total dose of 3 mg or Infusion 1 mg EPINEPHRINE in 250 ml of D5W (4 mcg/ml) IV infusion rate NTE (1-4 mcg/min).

FORMULATION KEY: (therapeutic classes and abbreviations used below)
1) Mu receptor antagonists (MU) (e.g. naloxone, naltrexone) Each member of this class has an accompanying designation indicating whether they are immediate acting or extended release (XR) (e.g. naltrexone and nalmefene are long acting MU antagonists, MUXR). Also note that this class can contain selective opioid receptor antagonists and agonists for kappa and delta subtypes.
2) A-1 Adrenergic receptor antagonists (A1ARA) (e.g. prazosin, tamsulosin) Each member of this class has an accompanying designation indicating whether they are selective (S) or non-selective (NS) for A1ARA subtypes 1A, 1 B, or 1D (e.g. Selective A 1 A receptor antagonist tamsulosin would be designated as S-A1ARA).
3) Anticholinergics (AC) (e.g. atropine, glycopyrrolate)
4) Paralytics/Muscle relaxants (PMR) (e.g. succinylcholine)
5) Respiratory Accelerants (RA) (e.g. Doxapram)
6) GABA Complex Antagonists (GCA) (e.g. flumazenil)
7) Anti-seizure/Membrane stabilizer (ASMS) (e.g. Dilantin XR)
8) Alpha2 agonists (A2A) (e.g., Clonidine)
9) Alpha 1 agonists (Al A) (e.g. phenylephrine —also listed as a “vasopressor” above)
10) Anticholinergic (AC) agents and/or cholinergic agents (C) (muscarinic receptor antagonist/anticholinergic, M3 receptor agonist or a nicotinic receptor general or selective agonist) (e.g. Pilocarpine)
11) Combined alpha-1 adrenergic antagonist and anticholinergic (AARA-AC or “COMBO”) (e.g.

Droperidol)
12) Beta Blockers (BETA B) (e.g. esmolol, atenolol, propranolol)

Specific combinations of compounds (Formula Equations) for use in embodiments provided herein include the following:
Representative IMMEDIATE REVERSAL NON-MEDICAL embodiments include: IRNM1, IRNM2, IRNM3, IRNM4, IRNM5.
Representative IMMEDIATE REVERSAL MEDICAL NO AW (IRMnAW) embodiments: (including all of the previous embodiments in addition can be administered as an alternative to these formulations): IRMnAW1, IRMnAW2, IRMnAW3.
Representative IMMEDIATE REVERSAL MEDICAL AW embodiments (these personnel can also employ formulations listed in MEDICAL NO AW) include: IRMAW1, IRMAW2.

Representative POLYSUBSTANCE embodiments include: Polyl, Poly2, Poly3.
Representative PROPHYLAXIS for ACTIVE Stimulant /IV USER embodiments include: PASOU1, PASOU2.
Representative PROPHYLAXIS for FIRST RESPONDERS embodiment include: PFR1.

### Example 2

### Methods for Fentanyl/Fentanyl Analog Overdose Treatment, Non-Medical Provider

The following combinations of therapeutic agents are appropriate for use by non-medically trained persons in an immediate reversal situation: IRNM1, IRNM2, IRNM3, IRNM4, IRNM5.

Representative Delivery systems for non-medical and medical providers: (e.g., intranasal and intramuscular injection). This description is intended to illustrate and be informative, but is not intended to be comprehensive regarding the scope of resuscitation from opioid and stimulant overdose, or regarding more sophisticated airway and cardiovascular treatment algorithms.

FIRMR/FIRE syndrome/Stimulant and Synthetic Opioid Induced Vascular Events (SSOIVE) REVERSAL AGENTS for resuscitation from opioid and stimulant overdose as a Nasal Spray is a prescription medicine used for the treatment of an opioid and/or stimulant overdose emergency such as an overdose or a possible suspected opioid overdose where fentanyl or fentanyl analogues and stimulants (e.g. methamphetamine, cocaine) are involved or if either of these drugs are combined with morphine derivatives and present with signs of breathing problems, sudden onset of muscle rigidity in chest wall, upper extremities and/or abdomen or “seizure-like”, rapid loss of consciousness, severe sleepiness, body found with syringe or tourniquet still in place/injection site, rapid onset of cyanosis, pinpoint pupils, or not being able to respond after an injection of illicit drugs or unintentional ingestion of fentanyl or fentanyl analogues. Similarly, signs or symptoms of major vascular events include cardiovascular (e.g. chest pain, chest tightness or “heaviness”, severe “indigestion”, pain radiating from chest to neck, jaw or L shoulder) and cerebrovascular (e.g. sudden weakness in face and/or one side or both sides of body, syncope or severe dizziness, visual field changes, severe headache).

FIRMR/FIRE syndrome/SSOIVE REVERSAL AGENTS—Nasal Spray is to be given right away, but does not take the place of emergency medical care. Get emergency medical—EMS CALL 911- help right away after giving the first dose of FIRMR/FIRE syndrome/SSOIVE REVERSAL AGENTS Nasal Spray, even if the person wakes up. The opioid effects often outlast the effect of the mu antagonist agent unless it is long acting. FIRMR/FIRE syndrome/SSOIVE REVERSAL AGENTS—Nasal Spray can be safe and effective in children for known or suspected opioid overdose however, but always refer to the package insert for dosing guidelines and call EMS-911 immediately. FIRMR/FIRE syndrome/SSOIVE REVERSAL AGENTS Nasal Spray is used to temporarily reverse the effects of opioid medicines and specifically opioid overdoses that involve fentanyl and fentanyl analogues. The medicine in FIRMR/FIRE syndrome/SSOIVE REVERSAL AGENTS Nasal Spray has little effect in people who are not taking opioid medicines, but can either raise or lower blood pressure and repeat dosing should be done with caution only in a witnessed overdose or wait till skilled emergency providers arrive. Always carry FIRMR/FIRE syndrome/SSOIVE REVERSAL AGENTS Nasal Spray with you in case of an opioid emergency. Use FIRMR/FIRE syndrome/SSOIVE REVERSAL AGENTS Nasal Spray right away if you or your caregiver think signs or symptoms of an opioid emergency are present, even if you are not sure, because an opioid emergency can cause severe injury or death.

REVERSAL AGENTS—Nasal Spray. Rescue breathing or CPR (cardiopulmonary resuscitation) and BLS (basic life support) may be given while waiting for emergency medical help.

The signs and symptoms of an opioid emergency can return after FIRMR/FIRE syndrome/SSOIVE REVERSAL AGENTS—Nasal Spray is given. If this happens, give another dose after 2 to 3 minutes using a new FIRMR/WCS REVERSAL AGENTS—Nasal Spray and closely watch the person until emergency help is received.

HOW TO USE FIRMR/WCS/SSOIVE REVERSAL AGENTS—Nasal Spray as delivery system

In opioid overdose emergencies recognize symptoms and taking prompt action is critical to potentially saving a life. If you suspect an opioid overdose, administer FIRMR/FIRE syndrome/SSOIVE REVERSAL AGENTS -Nasal Spray and get emergency medical assistance right away. Key steps to administering FIMR reversal agents—Nasal Spray:

1. Peel back the package to remove the device. Hold the device with your thumb on the bottom of the plunger and two fingers on the nozzle.

2. Place and hold the tip of the nozzle in either nostril until your fingers touch the bottom of the patient's nose.

3. Press the plunger firmly to release and inject the dose into the patient's nose.

The FIRMR/FIRE syndrome/SSOIVE REVERSAL DRUG-Auto-Injector is a disposable, pre-filled automatic injection device to be used in the event of an opioid emergency such as an overdose or a possible suspected opioid overdose where fentanyl or fentanyl analogues and stimulants (e.g. methamphetamine, cocaine) are involved or if either of these opioid drugs are combined with morphine derivatives. FIRMR/FIRE syndrome/SSOIVE REVERSAL DRUG-Auto-Injector administers NALOXONE and/or NALMEFENE and an ALPHA-1 ADRENERGIC RECEPTOR ANTAGONIST (in one specific example). Auto-injectors may be color-coded or otherwise readily labeled to acknowledge that they may contain other combinations of medications (see Table 1) to be used at the discretion of a medical provider and are to be used in the event of an opioid overdose where fentanyl or a fentanyl analogues are suspected. FIRMR/FIRE syndrome/SSOIVE REVERSAL DRUG-Auto-Injector is a prescription medicine used for the treatment of an opioid emergency such as an overdose or a possible suspected opioid overdose where fentanyl or fentanyl analogues are involved and present with signs as noted above in “Delivery systems for non-medical and medical providers”.

FIRMR/FIRE syndrome/SSOIVE REVERSAL DRUG-Auto-Injector is used to temporarily reverse the effects of opioid medicines and specifically opioid overdoses that involve fentanyl and fentanyl analogues. The medicine in FIRMR/FIRE syndrome/SSOIVE REVERSAL DRUG-Auto-Injector has little effect in people who are not taking opioid medicines, but can lower blood pressure and repeat dosing should be done with caution only in a witnessed overdose or wait till skilled emergency providers arrive. In the meantime, provide CPR and BLS support until emergency providers arrive. Get emergency medical help right away after giving the first dose of FIRMR/FIRE syndrome/SSOIVE REVERSAL DRUG-Auto-Injector Rescue breathing or CPR (cardiopulmonary resuscitation) and BLS (basic life support) may be given while waiting for emergency medical help. The signs and symptoms of an opioid emergency can return after FIRMR/FIRE syndrome/SSOIVE REVERSAL DRUG-Auto-Injector is given. If this happens, give another dose after 2 to 3 minutes using a new FIRMR/FIRE syndrome/SSOIVE REVERSAL DRUG-Auto-Injector and closely watch the person until emergency help is received.

### Example 3

### Methods for Combined Stimulant and Fentanyl/Fentanyl Analog Overdose Treatment, Medical Provider

The technology provided herein is designed to accommodate multiple types of first responders with different skill sets and training. TABLE 1 and the Formula Equations provided herein identify and assign combination compounds to each type of provider, including by clinical presentation. These FIRMR/FIRE syndrome/SSOIVE REVERSAL DRUGS should be combined in effect with standard BLS/CPR/ACLS protocols to manage the effects of opioid and/or stimulant overdose and used to temporarily reverse the effects of opioid and/or stimulant medicines and specifically opioid overdoses that involve stimulants and fentanyl and fentanyl analogues.

The following are exemplary situations in which a Provider who has medical training can administer the indicated combination therapy:
1) Suspected opioid and stimulant OD, unresponsive patient with rapid, bounding pulse indicating high BP:

Use formulation with Naloxone and/or Nalmefene, Prazosin OR A-1A selective antagonist and/or a Beta Blocker (BETA B) (e.g. esmolol, atenolol, propranolol).
2) Suspected opioid and stimulant OD with prominent rigidity: Use formulation with Naloxone and/or Nalmefene, Prazosin or A-1A selective antagonist and glycopyrrolate and/or atropine or a selective M3 agonist to block F/FA muscarinic antagonist effects and/or Succinylcholine or Rocuronium (e.g. only if medical provider has advanced AW management training and all necessary equipment available to secure the AW). Anticholinergics are to be avoided in the event of tachycardia or cardiac ventricular arrhythmias. 3) Uncertain presentation: In the event that the medical provider is uncertain of physiologic or clinical presentation in “Suspected Opioid Overdose”, use the baseline “Non-Medical” dosing kit until vital signs are apparent and directional, then follow algorithm as above in clinical scenarios 1-2.

More generally, the following is a list of preparations (that is, combinations of compounds) that are applicable for use by a medically trained Provider—with hemodynamic monitoring available and with or without airway monitoring/ equipment available (“Immediate Reversal Medical No AW”) or with the ability to monitor and equipment to manage airway function (“Immediate Reversal Medical AW”; can also employ all formulations listed in Immediate Reversal Medical No AW):
Immediate Reversal Medical No A W—composition combinations: IRMnAW1, IRMnAW2, IRMnAW3.
Immediate Reversal Medical AW—composition combinations: IRMAW1, IRMAW2
In each clinical presentation scenario, after administering a drug, the Medical provider should continue to provide CPR/ACLS and continue to reassess the patient every 1-2″ for response to the last drug given and assess clinical presentation for the next type of dose to be given. Re-dosing of drug combination can be done every 2-3 minutes (2-3″) up to four doses, or more if the patient is responding, but still needs additional reversal. Alternatively, Beta Blockers (BETA B) (e.g. esmolol, atenolol, propranolol) can be used in the event of severe tachycardia).

The clinical scenarios provided in “TABLE 1” and listed above serve as the guidelines for continued dosing strategies (e.g., if the pulse is slow HR<60, use the composition that contains ATROPINE or GLYCOPYRROLATE). Anticholinergics are to be avoided in the event of tachycardia or cardiac ventricular arrhythmias.

If patient presents with severe rigidity or stiffness and the Medical provider has no AW experience, they would use the compound with ATROPINE and Glycopyrrolate and and/or a selective M3 agonist to block F/FA muscarinic antagonist effects and upper AW effects and decrease “vagal tone” which will help improve rigidity. Anticholinergics are to be avoided in the event of tachycardia or cardiac ventricular arrhythmias.

If the patient presents with extreme rigidity and the Medical provider has AW training and AW equipment available, then use the compound that contains SUCCINYLCHOLINE to break the rigidity). Alternatively, a mu opioid receptor antagonist can be combined with an alpha 1-adrenergic antagonist and an anticholinergic agent and/or a selective M3 agonist to block F/FA muscarinic antagonist effects and upper AW effects.

In each scenario, after administering a drug combination, the Medical provider should provide CPR/ACLS and continue to reassess the patient for response to the last dose given, and assess clinical presentation for the next type of medication to be given. The Medical provider with AW training has the most options available followed by the Medical provider with only hemodynamic training.

### Example 4

### Methods for Polysubstance Overdose Treatment

In instances of suspected or known polysubstance overdose, treatment is carried out similarly to the description provided in the prior examples but using one of the following combined therapeutic compositions: Polyl, Poly2, Poly3.

### Example 5

### Methods of Prophylaxis for Habitual Stimulant Drug Users

In embodiments used to provide prophylaxis for habitual drug uses, dosing regimens, formulas, and general instructions are largely as presented in prior Examples. However, combinations of compounds for these embodiments have been modified to include long acting mu antagonists (e.g. Naltrexone, Nalmefene). This is because habitual users of illicit stimulants that are not using, seeking or intentionally trying to use opioids will be unlikely to feel the effects of mu antagonists unless the user inadvertently has been using a stimulant contaminated or mixed with fentanyl/fentanyl analogues. or This is contrasted with individuals with a severe Opiate use disorder (OUD) who are normally averse to taking any type of mu antagonist because it will readily precipitate moderate to severe withdrawal symptoms if the patient has not already undergone a formal opiate detoxification process for at least 5-7 days prior to the administration of a mu antagonist. In this embodiment, the combination therapeutic compounds are designed specifically for harm-reduction in a population that may knowingly or unknowingly expose themselves to the risk of FIRE syndrome and SSOIVE effects from F/FAs and stimulants that they are actively seeking and consuming. Appropriate compound combinations include: (PASOU1) MU (naltrexone or nalmefene)+5-A1 ARA+/−NS-A1ARA, (PASOU2) MU+S-A1ARA+NS-A1ARA+A2ARA. Beta Blockers (BETA B) (e.g. esmolol, atenolol, propranolol) can be added additionally for prevention or treatment of severe tachycardia.

### Example 6

### Assessment of the Efficacy of α-1 Adrenergic Antagonists and Mu Opioid Antagonists in Treating FIMR/FIRE Syndrome and/or Cardiovascular or Cerebrovascular Events Related to Stimulant Overdose or Combined Stimulant and Fentanyl/Fentanyl Analogue Overdose or Stimulant and Synthetic Opioid Induced Vascular Events (SSOIVE) from Opioid and Stimulant Overdose

This example describes methods for assessment of α-1 adrenergic antagonists and in their efficacy in preventing or reversing fentanyl induced muscular rigidity (FIMR), FIRMR and laryngospasm, FIRE syndrome and SSOIVE effects. Also described are methods for assessment of adjunctive reversal agents for prophylaxis and reversal of FIRMR/FIRE syndrome/Stimulant and Synthetic Opioid Induced Vascular Events (SSOIVE) in an animal model.

FIRE syndrome Animal Model: This experimental series will use an innovative animal (rat) model of FIRE syndrome and SSOIVE effects for validation of underlying physiologic mechanisms of FIRE syndrome and SSOIVE effects, specifically upper airway effects of F/FAs and FIRMR in order to test lead compounds for treatment of symptoms of toxic F/FA exposure or overdose or symptoms associated with stimulant overdose (cardiovascular effects such as myocardial infarction or arrhythmia, and/or cerebrovascular effects such as stroke or hypertensive crisis, tachycardia) and/or combined with fentanyl or a fentanyl analogue and/or the vascular effects or FIRE syndrome effects that may be enhanced by noradrenergic activities of either drug or the combination of stimulants with fentanyl/fentanyl analogues. The animal model will also allow for cardiac monitoring and invasive vascular monitoring to determine real time physiologic hemodynamic and cardiovascular effects and changes occurring from polysubstance combinations of stimulants with fentanyl/ fentanyl analogues.

Hypothesis 1: A new animal model with face validity for human VCC and FIRE syndrome can be used to identify and/or characterize lead compounds for F/FA toxicity.

Hypothesis 2: The combination of stimulants with the synthetic opioid fentanyl and its analogues leads to a more rapid and severe form of FIRE syndrome and SSOIVE and vascular effects due to the reinforcing effects of each drug on noradrenaline release. Noradrenaline release underlies catastrophic vascular effects of stimulants on the brain and heart and the catastrophic respiratory and vascular effects of fentanyl based drugs.

Rationale and Background: The key feature of F/FA-induced FIRE syndrome in humans is the rapid onset of respiratory failure with laryngospasm/ vocal cord closure (VCC) and loss of pulmonary compliance and FIRMR/FIRE syndrome (Scamman, Anesth Analg 62:332-334,1983) and appears to be the most likely cause of death from F/FA overdose(Somerville et al., MMWR 66:382-386. 2017). In fact, individuals with tracheostomies that bypass the vocal cords (VC), tolerate high dose F/FA without developing FIRE syndrome, demonstrating that VCC is the key feature of FIRE syndrome severity (Scamman, Anesth Analg 62:332-334,1983). VCC was documented in 28 of 30 human adult subjects using fiber optic visualization of the larynx with high dose F/FA (Bennet et aL, Anesthesiology 8(5):1070-1074, 1997). These studies indicate FIRE syndrome from F/FA exposure has a complex etiology, and that effective treatment development requires an innovative animal model for evaluation of potential therapeutic compounds, as previous animal models have not evaluated laryngeal and respiratory muscle function directly.

Additionally, the rising deaths caused by stimulants that have been adulterated with the synthetic opioids of the fentanyl family suggest that further investigation is needed to understand the potential additive and possible synergistic pharmacological effects that each class of drug may contribute to the rapid death seen with these drugs in combination.

The inventor describes a novel, experimental animal model to better replicate the clinical effects of human FIRE syndrome seen with fentanyl compounds and to monitor significant vascular changes and events caused by stimulants with the advantage of being able to assess the effects of each drug individually and in combination. This innovative model facilitates quantitative endoscopic video monitoring of the laryngeal aperture as a measure of VCC and upper airway changes, while using an anesthetic technique (e.g. Urethane 0.9-1.8 mg/kg and alpha-chloralose 40 mg/kg via intraperitoneal injection) and upright positioning that will optimize spontaneous respiration and minimally suppress airway reflexes. Most of the previous work with animal models of fentanyl induced muscle rigidity occurred prior to the definitive human study by demonstrating the key involvement of VCs in humans with FIRE syndrome induced by F/FA. These prior animal models bypassed VC with either endotracheal intubation or tracheostomy or left the VCs unobserved, therefore the direct effects of previous therapies on VC function and upper airway mechanical failure were unknown prior to the study design described here. There has been no definitive work on alpha 1 adrenoceptor or subtype antagonists in a FIRE syndrome animal model that includes the airway effects of fentanyl compounds and our preliminary data are the first effort to demonstrate the potential role of alpha 1 adrenoceptor subtypes in symptoms of F/FA toxic exposure or overdose. Similarly, no previous animal studies have looked at the degree of lethality or underlying pharmacologic mechanisms in rapid death resulting from combining stimulants and fentanyl/fentanyl analogues, specifically hemodynamic effects.

Experimental Design: Development of a rat airway monitoring model for lead compound identification for F/FA exposure is partially adapted from Yang et al., Anesthesiology, 77(1): 153-61, 1992; and Rackham, Neuropharmacology, 19(9):855-9, 1980. On the day of the procedure rats (male and female Sprague Dawley, 250-300 gm) will be administered ketamine (e.g. 80 mg/kg and xylazine 8 mg/kg, i.p.). Alternatively a dose of urethane 0.9-1.8 mg/kg and alpha-chloralose 40 mg/kg via intraperitoneal injection may be administered as an alternate anesthetic agent, as it is significantly longer in duration for circumstances when longer experimental observation is required, has no alpha 1 adrenergic receptor activity and minimal effects on airway secretions and upper airway visibility. Supplemental glycopyrrolate 0.5 mg/kg is administered 30 minutes prior to airway instrumentation and is used as an antisialagogue to minimize airway secretions and maximize airway and vocal visibility. After onset of surgical anesthesia verified by lack of response to 2 second paw pinch, animals will be immobilized on a rodent intubating stand or supine on a heated surgical table. Eyes are lubricated (for instance, with Lacri-Lube® eye gel) and temperature monitored using a rectal temperature probe placed prior to surgical vascular access procedures. PhysioSuite® monitors are placed on a paw for pulse oximetry oxygen saturation measurement, perfusion rate and heart rate. The temp probe is also monitored by the physio-suite device.

The skin of the lower abdomen is prepared by removing hair with an electric razor, and skin prepared in sterile fashion with alcohol swabs and povidone iodine swabs. A lower abdominal wall incision is made at the level of the inguinal ligament to expose the femoral artery and femoral vein. Each vessel is cannulated with sterile surgical tubing for arterial pressure monitoring from the femoral artery and vascular intravenous injection access for the femoral vein. An oral retractor is placed to displace the tongue from the airway and a 1 ml syringe barrel is placed midline in the oropharynx as an introducer guide for the 2.7 mm rigid endoscope to visualize epiglottis and vocal cords prior to injection of fentanyl. Once vocal cords are visualized, the video camera attached to the endoscope is activated to begin recording video images in real time prior to fentanyl injection and after injection for up to 10 minutes if the animal continues to demonstrate open vocal cords, persistent heart rate, oxygen saturation and respiratory rate.

Oxygenation is measured for instance using pulse oximetry, and respiratory rate is measured for instance by precordial chest auscultation of breath sounds with output to an audio recorder with a visual display. Cardiac function is measured using heart rate and hemodynamics will be measured continuously with invasive arterial catheter monitoring. The femoral artery and vein will be cannulated and can be used for blood samples, arterial pressure monitoring, and drug administration. Rectal temp will be kept at 37+/-0.5²C using a heat lamp and temperature controller. Adequate general anesthesia and analgesia is maintained to allow for invasive procedures, but to maintain spontaneous respiration to facilitate vocal cord visualization. The video endoscope will be positioned for continuous visualization of the larynx.

Electromyographic (EMG) signal will be acquired as described and adapted from previous work (Weinger et al., Brain Res, 669(1):10-8, 1995; Rackham, Neuropharmacology, 19(9): p. 855-9, 1980; Benthuysen et al., Anesthesiology, 64(4):440-6, 1986; Yadav et al., Int J Toxicol, 37(1):28-37, 2018) . Briefly, monopolar recording electrodes will be percutaneously inserted into the left gastrocnemius muscle and lateral abdominal wall and a ground electrode will be placed in the right hindlimb. As previously described, high dose F/FAs have a stereotypical EMG presentation of sustained isometric contraction from ongoing muscle fiber activity (Weinger et al., Brain Res, 669(1):10-8, 1995). The raw EMG signal will be amplified, filtered and recorded for 5 minutes before, and at least 30 minutes after administration of the test substance. Total EMG activity from each site will be averaged every 5 minutes for calculating the ED₁₀₀ and 95% confidence limits of each F/FA tested. Regression analysis will be used to calculate ED₅₀ and 95% confidence limits for reduction of rigidity from lead compounds tested.

Calculation of Dose response curve/ED₁₀₀ for F/FA VCC and WCS and ED₁₀₀ for methamphetamine and cocaine induction of myocardial ischemia and/or significant arterial pressure elevation (e.g., FIRE syndrome and SSOIVE). Rats will be randomized into experimental groups and we will estimate ED₁₀₀ for VCC and WCS for each F/FA. Rats will be randomized into experimental groups and we will estimate ED 100 for methamphetamine and cocaine induction of myocardial ischemia and/or significant arterial pressure elevation for each stimulant. Note LD₉₀ for methamphetamine 100 mg/kg and LD85 for cocaine as 70 mg/kg in the rat model as adapted from previous work (Derlet et al., Pharmacol Biochem Behav, 36(4): 745-9, 1990) F/FAs will be administered by infusion pump 10 mcg/kg/min or a comparable dose rate based on the potency of the analogue compared to fentanyl, from MOR binding studies. Carfentanil is 100X the relative potency so will be administered at 0.1 mcg/kg/min) until the animal demonstrates VCC (significant closure of glottis structures or appears to have airway obstruction) and/or FIRE syndrome. Similarly, stimulants will be administered by infusion pump in a dose range known to increase arterial pressure by 50% or more and/or an increase in arterial pressure until myocardial ischemia is demonstrated in 2 leads or more of EKG. Each analogue will be administered until 4 animals have consecutively demonstrated VCC and FIRE syndrome and similarly for vascular effects with stimulants. In the event that an analogue does not produce VCC in a test subject at a proportional dose to fentanyl, we will increase the baseline dose by 25% until a consistent effect of VCC is seen in 3 test subjects. Time to effect and dose will be recorded for VCC/FIRE syndrome and used to plot a dose response curve for each. Vital signs will be noted at the time of VCC and each analogue group will be monitored for 30 min for return of spontaneous respiration. If no return at the end of this time, the animal will receive a final bolus of both ketamine 200 mg/kg and fentanyl 20 mg/kg for euthanasia as adapted from previous work (Yadav et al., Int J Toxicol, 37(1):28-37, 2018).

Use of selective alpha 1 adrenergic receptor agonists/antagonists to demonstrate FIRE syndrome in vivo: Alpha 1 adrenergic subtype antagonists will be used to isolate each receptor subtype as previously described by Sohn et al., Anesthesiology, 103(2): 327-34, 2005. Alpha 1 subtypes (2 of 3 alpha 1 subtypes) will be antagonized and the third subtype will be agonized with NE, EPI , cocaine and methamphetamine until all combinations have been tested (Sohn et al., 2005). 29. Use of specific alpha 1 subtype antagonists in vivo to systematically and selectively isolate and block each subtype (1A: 5-Methylurapidil, 1B: chloroethylclonidine, 1 D: BMY 7378)29 and each combination of subtype (1A+1B , 1A+1D, 1 B+1 D). A range of physiologic NE doses will be administered to each group with isolated receptor subtypes)) EMG will be used, and direct view microscopy of the VCs will gauge the occurrence of acute airway closure and/or FIRE syndrome of respiratory muscles (>50% closure of laryngeal aperture with O₂sat <94% and end tidal CO₂>50 mmHg, EMG value sustained contraction >50% of baseline for 5 minutes).

Preclinical drug characterizations and lead molecule identification in animal model of FIRE syndrome and SSOIVE. A series of alpha 1 adrenoceptor antagonists, alpha 2 adrenoceptor agonists , opioid receptor antagonists and/or cholinergic agents as described in formulations noted above, will be administered in a dose range and at different time points after F/FA and stimulant IV administration to establish which agents may be effective in the reversal of FIRE syndrome and SSOIVE or components of FIRE syndrome (chest wall/diaphragm rigidity (FIRMR) and VCC, or SSOIVE cardiovascular or cerebrovascular events related to stimulant overdose or combined stimulant and fentanyl/fentanyl analogue overdose. cardiovascular compromise) and may have clinical utility for F/FA and stimulant toxic exposure and/or overdose. Each reversal agent will be administered at several time points (e.g. given at Time 0, T+1. . . T+10 etc.) following each individual F/FA and stimulant administration and combinations of F/FA and stimulant doses to identify lead compounds that can reverse or antagonize WCS or cardiovascular or cerebrovascular events related to stimulant overdose or combined stimulant and fentanyl/fentanyl analogue overdose.

Proposed Drugs and doses tested: 1) Non-selective antagonist: prazosin, 1-500 mcg/kg or 50,100, 250 mcg/kg; 2) terazosin 10-200 mcg /kg or 70, 200 mcg/kg; 3) selective antagonist: tamsulosin 1-10 mcg/kg or 5, 10 mcg/kg; 3) Alpha 2 agonist: clonidine, 1-200 mcg/kg or 35, 175 mcg/kg; MOR antagonists: 1) naloxone 0.01-1 mg/kg or 0.1, 0.5, 1 mg/kg; 2) nalmefene 1-100 mcg/kg or 25, 50, 100 mcg/kg; 3) naltrexone 0.1-1.0 mg/kg or 0.35, 0.7, 1.0 mg/kg; Cholinergic agents: 1) Atropine 0.05-1 mcg/kg 2) Glycopyrrolate 1-4 mcg/kg ;3) pilocarpine 0.015-0.05 mcg/kg and other muscarinic agonists; 4) Nicotine; and/or other nicotinic agonists (0.1-2 mg/kg). Combinations will be determined based on efficacies in the rat model; 5) Beta Blockers (BETA B) (e.g. esmolol, atenolol, propranolol) esmolol 0.01-0.5 mg/kg.

Timing: Drugs will be administered at intervals between 1-10 minutes after F/FA and stimulant administration. These time points may be expanded, for instance to include T minus 60 minutes (T-60), T-45, T-30, T-15 T-10, and so forth. Simultaneous administration of F/FAs in various combinations with the agents listed herein will be used to assess their potential for the development of opioid analgesic agents (e.g. F/FAs) with modified side effect profiles (e.g. respiratory depression, laryngospasm, FIRMR, FIRE syndrome and SSOIVE, addiction etc.) and thereby enhance or increase the safety margin and potential for extended ranges of analgesia.

Lead compounds will be defined as: Reversal of VCC/laryngeal aperture by 50% or more, O₂saturation is greater than or equal to 94% and end tidal CO₂ is less than 50 mmHg, and reversal of rigidity as measured by EMG is 50% or more from F/FA effects, and modified from Bennett et al., Anesthesiology, 87(5): 1070-4, 1987; and Weinger et al., Brain Res, 669(1): 10-8, 1995. Reversal of arterial pressure elevation to within 25% of baseline and reversal of ischemia as noted by EKG.

Data Analysis: We will plot dose response curves and timing of response for each analogue. Data from the experiments will be analyzed individually. For each drug, a two-way ANOVA will be performed to evaluate the effect of drug dose (between-subject factor) on EMG, VCC, FIRE syndrome and SSOIVE, and blood pressure over time (within-subject factor). This will be followed by Newman-Keuls a posteriori tests to assess dose effects at individual time points as well as differences in EMG activity over time within each dose group (Willette et al., J Pharmacol Methods, 17(1):15-25, 1987; Willette et al., Eur J Pharmacol, 91(2-3):181-8, 1983). Data will be expressed as mean +S.E.M., a p <0.05 will be considered to be statistically significant as adapted from Weinger et al. (Brain Res, 669(1):10-8, 1995).

Expected Results: The objective of this study is to identify drugs that can be used in combination to either reverse or prophylax against FIRE syndrome and SSOIVE caused by fentanyl/fentanyl analogues, the vascular and CNS effects caused by stimulants and the combined effect of these two classes of drugs when administered together in situations of overdose and/or toxic exposure and for the development of F/FAs with limited FIRE syndrome and SSOIVE side effects risk. It is believed that VCC with high dose F/FAs will be a prominent feature of the clinical presentation in the animal model, as seen in humans. Rats and humans have similar anatomic innervation of VCs by the vagus nerve from the medulla and the receptor distributions of alpha-1 adrenergic receptors, cholinergic and opioid receptors in the CNS indicating that this model will predict effective therapeutic agents that can be successfully trialed in humans for the treatment of F/FA induced FIRE syndrome and SSOIVE and respiratory depression. Additionally, severe arterial hypertension, myocardial ischemia and possibly CNS seizure activity will be prominent in this model. Data obtained from the herein-described experiments will provide dose response curves with the drugs tested that will predict effective/ therapeutic drug dosing ranges and drug combinations to prevent FIRMR/ laryngospasm FIRE syndrome and Stimulant and Synthetic Opioid Induced Vascular Events (SSOIVE) in these animals and similarly in humans. This will provide a model for future analogue testing and targeted drug development.

Some drug combinations are expected to be more or less effective in a particular dosing vehicle. Thus, different delivery modes, escalating dose regimens, and multiple/concurrent modes of delivery will be explored in this model to increase efficacy (e.g. inhaler, nebulizer, ophthalmic (10C), PO, sublingual or nasal delivery, IM, IO, IV etc.). These studies will provide lead molecules for treating and/or preventing FIRE syndrome (FIRMR and laryngospasm), Stimulant and Synthetic Opioid Induced Vascular Events (SSOIVE) and respiratory depression resulting from F/FA and/or Stimulant and Synthetic Opioid overdose or toxic exposure and/or F/FAs combined with morphine derived alkaloids (heroin) and to identify drugs that can be used in combination to either reverse or prophylax against FIRE syndrome and SSOIVE caused by fentanyl/fentanyl analogues, the vascular and CNS effects caused by stimulants and the combined effect of these two classes of drugs when administered together in situations of overdose and/or toxic exposure.

### Example 7

### Experiments & Clinical Trials

This Example provides brief descriptions of studies that will provide additional data related to the herein described technology.

Once the “affinity binding” and “animal studies” have established lead compounds, the compounds will be tested for safety in animals and an FDA IND application will be filed for testing in human subjects. Two sets of human clinical trials are described.

TRIAL A—HUMAN EMERGENCY ROOM and/or EMS Paramedic REVERSAL DRUG TRIAL for drug overdose with F/FA and stimulants to treat FIRE syndrome and SSOIVE in combined F/FA/Stimulant Overdose:

Trial A (EMS model) Rational: Acute opioid overdose presents as profound respiratory depression (RD) with anoxia that can lead to death. Administration of the mu opioid receptor antagonist naloxone to reverse RD has become the standard of care as part of out-of-hospital management of opioid overdose (Wanger et al., Acad Emerg Med. 5(4):293-9, 1998). However, in addition to RD, high doses of synthetic opioids, specifically fentanyl and fentanyl analogues (F/FA), also cause Wooden Chest Syndrome (WCS), a clinical presentation consisting of rapid vocal cord closure (laryngospasm; VCC) and severe diaphragm and chest wall rigidity that is often fatal without invasive airway management (Grell et aL, Anesth Analg 49(4):523-532, 1970; Bennett et al., Anesthesiology 87(5):1070-1074, 1997). Heroin, an opioid that is metabolized to morphine, is far less potent than F/FAs and is not known to cause WCS or FIRE syndrome.

A similar model can be applied to a SAFE INJECTION-type site (e.g. Insite-Vancouver BC) where individuals can go to a clearly disclosed location at which to safely inject illicit drugs under medical supervision as a harm reduction measure. These sites provide clean medical supplies for injection and medical staff to monitor the individual for overdose or other adverse reaction. In the event of an overdose, the medical professional can administer naloxone, oxygen, airway support and call 911 for medical transport to hospital if/as needed. In this case, individuals can be consented for participation in the study on their arrival interview/check and randomized to receive naloxone or “naloxone+”. (e.g. alpha 1 adrenergic antagonist combined with a mu opioid receptor antagonist, and/or additional beta-blocker). In this fashion, a trained medical professional can administer the control drug or test drug as part of the study design to participants that overdose. As part of the study, a serum sample can be drawn, and a sample of the drug could be provided for chemical analysis for participants requiring administration of a reversal drug and resuscitation support.

Additionally and similar to the significant rise in synthetic opioids reviewed above, synthetic stimulants (e.g. amphetamines, methamphetamine) and plant alkaloids (e.g. cocaine) have shown a significant increase in overdose deaths from 2010 to the present with deaths from methamphetamine overdose increasing from 1,400 in 2010 to well over 10,000 in 2017 and similar increases for cocaine at 14,000 in 2017 compared with 3000 in 2010. Of particular significance is the fact that these numbers mirror the rise in overdoses from fentanyl at 29,000 in 2017 versus 1000 in 2010 in the same time frame and that recent toxicology reports from these stimulant overdose deaths confirm that -70% of the stimulant overdoses show positive for fentanyl, carfentanil or other potent fentanyl analogues (Vestal, As the Opioid Crisis Peaks, Meth and Cocaine Deaths Explode, Stateline, Pew Charitable Trust, May 13, 2019; available online at pewtrusts.org/en/research-and-analysis/blogs/stateline).

The problem is that the synthetic opioid fentanyl and its analogues appear to significantly augment the lethality of stimulants and are under-recognized contaminants for which there are currently no molecules or compounds that exist or have been designed for reduction of death associated with their combination. There are no reversal or prophylaxis drugs or compounds for Stimulant and Synthetic Opioid Induced Vascular Events (SSOIVE) for resuscitation from or prevention of opioid and stimulant overdose. Considering that death rates from stimulants contaminated with fentanyl/fentanyl analogues represent ˜70% of lethal overdoses from stimulants, there is an urgent need to develop drugs or compounds that can increase survival rates and decrease the risk of death associated with the combinations of these drugs. Prior to the current opioid crisis, the combination or combining of fentanyl/ fentanyl analogues with stimulants was previously unknown and prior to disclosure in this document, no treatments with prophylaxis or reversal agents have previously been described for this combination of drugs. The purpose and intention of this trial design is to better identify the clinical features of overdoses from stimulants combined with synthetic opioids of the fentanyl class in the acute overdose setting and identify compounds that may increase survival rates.

FIRE syndrome and SSOIVE appears to be the key cause of rapid death and escalating numbers of death in the current F/FA driven opioid crisis, however, individuals who suffer from stimulant and polysubstance abuse that combines the synthetic opioid fentanyl or a comparable analogue intentionally or unintentionally with stimulants (e.g. methamphetamine, cocaine), they appear to have increased mortality compared with either agent alone. The lethal effects of either drug appears to be augmented by modulation of norepinephrine levels by each drug and directly relate to the underlying pharmacologic mechanisms whereby each drug has lethal effects on vascular and respiratory systems.

The overall objective of the study design is to determine whether naloxone+administered IV or IM and/or IN to out-of-hospital patients with suspected F/FA opioid overdose, is more effective at returning functional respiratory mechanics (resolution of respiratory depression and FIRE syndrome) to increase survival rates in F/FA overdose patients over the control treatment-naloxone.

Once the “animal studies” have established lead compounds, the compounds will be tested for safety in animals and an FDA IND application will be filed for testing in human subjects. Two sets of human clinical trials are described.

After full review and IRB approval of the study protocol and FDA IND approval of all test compounds, the institution(s) sponsoring the trial, HUMAN “Trial A” will begin recruitment of patients presenting with suspected acute stimulant and synthetic opioid overdose in an EMS field setting where participants will be randomized to receive an opioid reversal dose protocol that may include: 1) a Mu receptor antagonist and routine pharmacological support for hypertensive crisis and/or a cardiovascular event and/or a CNS event such as seizure or stroke OR 2) a Mu receptor antagonist and an α1 Adrenergic Receptor Antagonist (A1ARA) and/or a combination of “selective” and “non-selective” A1ARAs for treatment of a suspected , acute stimulant and synthetic opioid (fentanyl) overdose in patients that are suspected of or have a clinical presentation indicative of fentanyl or fentanyl analogues related overdose (e.g. rapid loss of consciousness after injection, rapid onset of cyanosis, chest and upper body rigidity, multiple doses of naloxone used and little or no response, needles and tourniquet still found in/on arm, sudden onset of rigidity or “seizure-like” activity after injection etc.) and a stimulant overdose (e.g. severe hypertension, seizure, evidence of a neurologic event such as stroke or a myocardial event with ischemia or an arrhythmia). These individuals will be randomized to receive either naloxone (e.g. the current standard of care) or will receive Naloxone+ given as a multi-component reversal agent as described herein, including a Mu antagonist, an al Adrenergic Receptor Antagonist (A1ARA) or a combination of “selective” and “non-selective” A1ARAs. Beta Blockers (BETA B) (e.g. esmolol, atenolol, propranolol).

The success of resuscitation will be measured by indicators of reversal such as the return of spontaneous respiration with adequate tidal volumes to sustain O₂Saturations >94% with room air or 1-4 L supplemental O₂, ease of assisted ventilation, resolution of muscular rigidity, the return of consciousness and responsiveness as gauged by the Glasgow Coma Scale. Blood samples will be drawn and analyzed for the presence of fentanyl or fentanyl analogues and metabolites such as norfentanyl.

Successful resuscitation will also be gauged by a return to physiologically normal hemodynamics and neurologic function. This data will be blinded and analyzed and compared with medical records of resuscitation to evaluate for statistical evidence of more rapid resuscitation and degree of re-normalization of hemodynamics, respiratory and neurologic function in individuals suspected of acute stimulant and synthetic (fentanyl) opioid overdose arriving in ER or being medically treated in the field by EMS or paramedic staff for medical treatment and receiving either current standard of care or a Naloxone+. One of the expected outcomes will be that individuals who are serologically confirmed to have significant serum levels of F/FAs and stimulants will show a response to treatment with the agent designated as Naloxone+ after no response or little response to multiple doses of the single agent naloxone.

Methods: As adapted from prior clinical studies of naloxone in the opioid overdose from Wanger et al., Acad Emerg Med. 5(4):293-9, 1998 and Sabzghabaee et al., Arch Med Sci 10(2):309-14, 2014, a multi-center, double blind, randomized control/non-placebo, additive trial of approximately 200 out-of-hospital patients, individuals suspected of acute stimulant and synthetic (fentanyl) opioid overdose. The trial will be conducted in several urban-out of hospital settings of high endemic areas for stimulant and synthetic opioid (fentanyl/F/FA) overdose. Time intervals will be compared, from arrival of EMS/paramedics at patient's side to time to response of adequate oxygenation and ventilation (defined as respiratory rate >/=10 breaths per minute with pulse oximetry values >/=94%), presence or absence of muscle rigidity, resolution or improvement of muscle rigidity, return of normal pulmonary compliance as measured by bag-valve-mask ventilation and duration of assistance, vital signs (blood pressure, heart rate and respiratory rate) return of physiologically normal ranges of hemodynamics (e.g. HR 50-100, BP 100-150/60-100) and change in level of consciousness (Glasgow Coma Scale and descriptive scale “comatose, obtunded, lethargic or conscious”). EMS providers called to the scene of an opioid overdose, while en route, will randomize the patient to receive a color coded vial for IV injection or a color coded IM injector containing either Naloxone or Naloxone+. Once the color coded IV med vial or IM injector is assigned, the same color code will be administered for the duration of the rescue study protocol. EMS/paramedic staff will follow the most current standards of care regarding resuscitation of opioid reversal and BLS and ACLS protocols and will administer the assigned medications within these parameters. Importantly, although it is not expected that the study protocol will deviate from these care standards, adherence to BLS and ACLS standards will always take precedence over the study protocol. Overdose victims will receive up to 3 doses IV/IM of the assigned drug(s) at 3 minute intervals and will be assessed for adequacy of respiration and oxygenation and presence/absence of muscle rigidity, while airway and hemodynamic management is provided. If the patient remains unresponsive and/or hypoxemic or has persistent muscle rigidity after 3 doses, the study protocol indicates immediate rapid sequence induction and securing airway via endotracheal intubation on transport to the hospital ER. In the event of potential aspiration or other airway complications requiring immediate intubation, airway management will take precedence over the study protocol. After patient has been stabilized, serum samples will be drawn for F/FA and drug analysis. Data will be reviewed and analyzed for statistical significance and efficacy of Naloxone+ in reversal of WCS and RD during opioid overdose with F/FAs and/or MDAs, compared to naloxone.

Halfway through the study period at 12 months (˜24 months total duration and ˜200 participants), color codes for the trial drugs will be crossed over. A preliminary data analysis will be performed at that time and if necessary, the protocol will be modified to either lower or increase the dose of Naloxone+ as long as side effects are minimal and the therapeutic efficacy has the potential of improvement with a dose adjustment.

Trial A: Participant recruitment: After IRB approval of the study protocol and FDA IND approval of the test compound/s, patients will be selected/recruited to the study based on the need for life-threatening and emergent treatment for individuals suspected of acute stimulant and synthetic (fentanyl) opioid overdose and all IRB criteria. All patients will be treated with the current standard of care for opioid overdose reversal, the mu opioid receptor antagonist, naloxone. Patients may be randomized to receive the additive experimental treatment for individuals suspected of acute stimulant and synthetic (fentanyl) opioid overdose. The dose of the additive drug will be in a range and/or combination that has been demonstrated to have a minimal side effect profile in adult humans as per existing and IND human safety study data.

Trial A: Population and setting: The study trial will involve adult patients 18 or older requiring EMS services for a suspected or reported opioid drug overdose and will be based in large urban areas where F/FAs represent a significant proportion (>60%) of all opioid and stimulant drug overdoses (e.g. Boston, Miami, Cincinnati, Buffalo). Alternatively, subjects may be recruited from SAFE INJECTION site(s) (such as Insite-Vancouver, BC), where individuals can go to a clearly disclosed location where they can safely inject illicit drugs under medical supervision as a harm reduction measure.

Trial A: AIM 3 Data collection/Data Sources: In addition to the standardized forms used by paramedic staff for documentation of emergency medical management, data collection for the study to track AIM 3 primary and secondary will be performed by paramedic/EMS staff via a standardized series of data management forms designed for visual clarity and binary “yes” or “no” answer format to record data specific to stimulant and synthetic (fentanyl) opioid overdose. Administration times for drugs will be preceded and followed by specific and systematic assessments of vital signs and quantitative and qualitative clinical measures defined below in Study Measures. The data chart will be organized in groups, color coded for each dose administered with an assessment section for each dose, in a flow chart that follows the physiologic course of opioid overdose reversal and/or emergency resuscitation. A side column will be present on the right side of the data sheet to note if ACLS or BLS is being performed at that time or for that assessment. Chart information on demographics and any known or preexisting health history will be noted by paramedic staff after the resuscitation is complete and/or patient care has been transferred to other medical providers or hospital/ER staff. We will obtain client name; date of birth; hospital record number, Medicaid number (if applicable); relevant medical history; primary, secondary and tertiary substance use problem (e.g. heroin, other opiates, fentanyl and other synthetic opioids, alcohol, cocaine, methamphetamine, cannabis etc.); age of first use, frequency of use, route of administration, and awareness of F/FA if present in serum drug screen. All records and data will be stored in a HIPAA compliant fashion. An extensive data encryption plan will be reviewed and approved by IRB and IT committees of participating hospitals or EMS service units prior to implementation of the study or the collection of patient data.

Trial A: Study Measures: The physical signs and symptoms associated with acute stimulant and synthetic (fentanyl) opioid overdose and morphine derived alkaloids (respiratory depression—RD) will be measured. Specifically, time intervals will be compared from arrival of EMS/paramedics at patient's side to development of/time to response of adequate oxygenation and ventilation (respiratory rate >/=10 breaths per minute with pulse oximetry values>/=94%), presence or absence of muscle rigidity, resolution or improvement of muscle rigidity, if present, normal pulmonary compliance as measured by bag-valve-mask ventilation and duration of assistance, vital signs (blood pressure, heart rate and respiratory rate) return of physiologically normal ranges of hemodynamics (e.g. HR 50-100, BP 100-150/60-100) and change in level of consciousness (Glasgow Coma Scale and descriptive scale). Time to return of spontaneous respiration, time to return of adequate respiration will be noted as described above. If muscle rigidity is present, the number of muscle groups involved (0- no rigidity, 1-jaw, neck, 2-shoulders, upper extremities, 3-chest wall, abdomen, 4-lower extremities) will be noted as per the grading system. If the EMS team is providing assisted ventilation, they will note and grade the ventilation effort required to maintain adequate oxygenation (0-easy or with 1-some effort or 2-difficult or 3-impossible to mask ventilate). Level of consciousness will be evaluated as per the Glasgow Coma Scale where the rating scale is defined as: Insert GCS scale rating system or can say refer to since it is well known.

The patient will receive up to 3 doses of naloxone/naloxone+. Resolution, inhibition or no change in symptoms will be noted 1 minute after each drug administration and prior to the next administration until patient either stabilizes with adequate respiration and oxygenation or requires intubation. The time to return for spontaneous respiration/ventilation and adequate oxygenation and the resolution of muscle rigidity will be analyzed for each group/drug. After 3 doses as per the study protocol, if persistent inadequate respiration and oxygenation is noted or if patient is unstable and requires immediate airway management, the patient will be intubated, and the time of induction and intubation will be recorded. Serum samples will be drawn for drug analysis and time of draw noted by EMS/Paramedic team.

Primary Outcome: Time to return to spontaneous respiration/ventilation with adequate oxygenation and resolution of muscle rigidity/FIRMR/laryngospasm/WCS and return of physiologically normal ranges of hemodynamics (e.g. HR 50-100, BP 100-150/60-100). Secondary Outcome: Level of consciousness. An overall goal of the study is identification of optimal lead compound efficacy in humans for treating the physical signs and symptoms associated with acute stimulant and synthetic (fentanyl) opioid overdose and reversal of F/FA overdose compared to naloxone and standard therapies and routine pharmacological support for hypertensive crisis and/or a cardiovascular event and/or a CNS event such as seizure or stroke and a clinical presentation indicative of fentanyl or fentanyl analogues related overdose (e.g. rapid loss of consciousness after injection, rapid onset of cyanosis, chest and upper body rigidity, multiple doses of naloxone used and little or no response, needles and tourniquet still found in/on arm, sudden onset of rigidity or “seizure-like” activity after injection etc.) and a stimulant overdose (e.g. severe hypertension, seizure, evidence of a neurologic event such as stroke or a myocardial event with ischemia or an arrhythmia).

Trial A: Intervention Power Analysis: Data will be analyzed by making comparisons of mean time intervals using an unpaired t-test and verified with nonparametric testing. Power calculations using the results from the control arm of the study will be performed using an a=0.05, power=0.90, A=2.0 minutes (to return of adequate spontaneous ventilation/oxygenation as previously described above) and SD=4.18. Alternatively the “A” variable could be defined as the number of doses required for adequate spontaneous ventilation/oxygenation as a marker of superiority of treatment (e.g. 1-2 doses of N+ with no ETT placed vs. 2-3 doses of N and ETT placed) and/or the return of physiologically normal ranges of hemodynamics (e.g. HR 50-100, BP 100-150/60-100) and neurologic functioning. Based on these preliminary calculations and comparable studies assessing EMS use of naloxone in emergency treatment of opioid overdose, a sample size of 184 (92 per arm) will be required (Wanger et al., Acad Emerg Med. 5(4):293-9, 1998 and Sabzghabaee et al., Arch Med Sci. 10(2):309-14, 2014).

Trial A: Analytical Methods and Sample Size Determinations: Field data forms will be reviewed on a weekly basis to assure appropriate application of suspected overdose protocol, data collection and review of vital signs and clinical charting. Specifically we will review drug combinations used by the patient , assure that vital signs are initially recorded and then every 2-3 minutes after, time of medication doses recorded, dose and route of administration of intervention drug, duration of basic airway intervention and tools used (bag valve mask-BVM/ oropharyngeal AW- OPA), total time from drug administration to return of adequate spontaneous ventilation/oxygenation (>10 BPM and O₂sat >93%) and a return to normal hemodynamics (e.g. normal blood pressure and sinus rhythm).

Primary Outcome: Time to return and number of doses required to return to spontaneous respiration and ventilation with adequate oxygenation and resolution of muscle rigidity/FIRMR/laryngospasm/FIRE syndrome and SSOIVE will be measured. Secondary Outcome: Level of consciousness with return of GCS score to (Decide on GCS score). Overall goal of the study is identification of optimal lead compound efficacy in humans for treating WCS and RD in reversal of F/FA overdose compared to naloxone. As adapted from Wanger et al., Acad Emerg Med. 5(4):293-9, 1998 and Sabzghabaee et al., Arch Med Sci. 10(2):309-14, 2014.

Trial A: Expected Results: “Naloxone (+)” is expected to perform as well if not better than naloxone in antagonizing morphine derived alkaloid induced respiratory depression and will be superior for antagonizing F/FA-induced FIRE syndrome and SSOIVE. The purpose and intention of this trial design is to better identify the clinical features of overdoses from stimulants combined with synthetic opioids of the fentanyl class in the acute overdose setting and identify compounds that may increase survival rates.

FIRE syndrome and SSOIVE appears to be the key cause of rapid death and escalating numbers of death in the current F/FA driven opioid crisis, however, individuals who suffer from stimulant and polysubstance abuse that combines the synthetic opioid fentanyl or a comparable analogue intentionally or unintentionally with stimulants (e.g. methamphetamine, cocaine), they appear to have increased mortality compared with either agent alone. The lethal effects of either drug appear to be augmented by modulation of norepinephrine levels by each drug and directly relate to the underlying pharmacologic mechanisms whereby each drug has lethal effects on vascular and respiratory systems. I predict that patients receiving naloxone+ will be less likely to require intubation/ invasive airway management and multiple doses of medication before primary and secondary outcomes are achieved. I also predict that overall mortality and morbidity will decrease and the survival rate will be significantly improved for naloxone+ patients who overdosed or were exposed to F/FAs or a combination of F/FAs with morphine derived alkaloids, and or any of these drug combinations that include methamphetamine, cocaine or stimulants.

HUMAN “TRIAL B” (CLINIC PROPHYLAXIS MODEL DRUG TRIAL):

Trial B (CLINIC PROPHYLAXIS TRIAL model) Rational:

Currently there are no medically assisted treatment drugs for stimulant use disorder. Although naltrexone has been tried for methamphetamine and cocaine users to control craving. However, the significant rise in synthetic opioids (fentanyls), synthetic stimulants (e.g. amphetamines, methamphetamine) and plant alkaloids (e.g. cocaine) as reviewed above, have shown a significant increase in overdose deaths from 2010 to the present with deaths from methamphetamine overdose increasing from 1,400 in 2010 to well over 10,000 in 2017 and similar increases for cocaine at 14,000 in 2017 compared with 3000 in 2010. These numbers mirror the rise in overdoses from fentanyl at ˜29,000 in 2017 versus 1000 in 2010 in the same time frame and that recent toxicology reports from these stimulant overdose deaths confirm that ˜70% of the stimulant overdoses show positive for fentanyl, carfentanil or other potent fentanyl analogues (Vestal, As the Opioid Crisis Peaks, Meth and Cocaine Deaths Explode, Stateline, Pew Charitable Trust, May 13, 2019; available online at pewtrusts.org/en/research-and-analysis/blogs/stateline). The problem, simply stated, is that the synthetic opioid fentanyl and its analogues appear to significantly augment the lethality of stimulants and are under-recognized contaminants for which there are currently no molecules or compounds that exist or have been designed for reduction of death associated with their combination. There are no reversal or prophylaxis drugs or compounds for Stimulant and Synthetic Opioid Induced Vascular Events (SSOIVE) for resuscitation from or prevention of opioid and stimulant overdose. Considering that death rates from stimulants contaminated with fentanyl/fentanyl analogues represent ˜70% of lethal overdoses from stimulants, there is an urgent need to develop drugs or compounds that can increase survival rates and decrease the risk of death associated with the combinations of these drugs. Prior to the current opioid crisis, the combination or combining of fentanyl/fentanyl analogues with stimulants was previously unknown and prior to disclosure in this document, no treatments with prophylaxis or reversal agents have previously been described for this combination of drugs. The purpose and intention of this trial design is to provide prophylaxis compounds to active users of stimulants who may be coming into consistent contact with synthetic fentanyl opioids for the purpose of decreasing risk and severity of overdoses from stimulants combined with synthetic opioids of the fentanyl class and identify compounds that may increase long term survival rates.

WCS appears to be the key cause of rapid death and escalating numbers of death in the current F/FA driven opioid crisis, however, individuals who suffer from stimulant and polysubstance abuse that combines the synthetic opioid fentanyl or a comparable analogue intentionally or unintentionally with stimulants (e.g. methamphetamine, cocaine), they appear to have increased mortality compared with either agent alone. The lethal effects of either drug appears to be augmented by modulation of norepinephrine levels by each drug and directly relate to the underlying pharmacologic mechanisms whereby each drug has lethal effects on vascular and respiratory systems.

The overall objective of the study design is to determine whether a long acting mu opioid receptor antagonist such as naltrexone or nalmefene administered orally or sublingually in combination with an al Adrenergic Receptor Antagonist (A1ARA) and/or a combination of “selective” and “non-selective” A1ARAs for overdose prophylaxis in active users of stimulant and synthetic opioids (fentanyls) will impact long term survivability and survival rates compared to groups that are not treated.

Once the “animal studies” have established lead compounds, the compounds will be tested for safety in animals and an FDA IND application will be filed for testing in human subjects. Two sets of human clinical trials are described.

After full review and IRB approval of the study protocol and FDA IND approval of all test compounds, the institution(s) sponsoring the trial, HUMAN “Trial B” will begin recruitment of ˜200 adult patients 18-50 with stimulant use disorder. The participants will be consented and screened for the past 30 day use of methamphetamine and/or cocaine. Screeners who have had at least 10 days of use in the last 30 days are eligible. Patients who are actively taking opioids for pain medication, actively seeking out illicit fentanyl or have planned upcoming medical procedures requiring extended use of pain medications will be disqualified from participation. Individuals who screen positive will have their UDS further screened for the presence of fentanyl and fentanyl analogues. If positive on screening they will qualify for trial. Patients will be randomized to either receive active treatment or placebo for a 6 month trial. Trial subjects and researchers will be blinded to all treatments administered. Subjects will be interviewed and screened 2x /week and will be compensated for visits and participation. At the end of the 6 month trial, patients will be given the option of continuing on open label medication for another 6 month cycle with visits reduced to 2x /month and will continue to be compensated and tracked as in the first blinded trial period. Subjects will be asked to report any episodes of overdose or suspicion of overdose during trial periods. Subjects will be educated on the clinical signs and symptoms of a suspected stimulant and synthetic opioid (fentanyl) overdose, The clinical presentation education will include detailed training sessions how to recognize fentanyl or fentanyl analogues related overdose (e.g. rapid loss of consciousness after injection, rapid onset of cyanosis, chest and upper body rigidity, multiple doses of naloxone used and little or no response, needles and tourniquet still found in/on arm, sudden onset of rigidity or “seizure-like” activity after injection etc.) and a stimulant overdose (e.g. severe hypertension, seizure, evidence of a neurologic event such as stroke or a myocardial event with ischemia or an arrhythmia).

Methods: As adapted from prior clinical studies of naloxone and naltrexone in the opioid overdose (e.g., Parmar et al., Addiction. 112(3):502-515. 2016; Murphy et al., Addiction. 112(8):1440-1450. 2017; Larochelle et al., Ann Intern Med. 169(3):137-145, 2018), a multi-center, double blind, randomized control/placebo, trial of approximately 200 out-of-hospital patients, of individuals with stimulant use disorder suspected of and with objective evidence of stimulant and synthetic (fentanyl) opioid exposure and use. The trial will be conducted in several urban-out of hospital settings of high endemic areas for stimulant and synthetic opioid (fentanyl/F/FA) overdose.

Halfway through the study period at 12 months (˜24 months total duration and ˜200 participants), color codes for the trial drugs will be crossed over. A preliminary data analysis will be performed at that time and if necessary, the protocol will be modified to either lower or increase the dose of Naloxone+ as long as side effects are minimal and the therapeutic efficacy has the potential of improvement with a dose adjustment.

Trial B: Participant recruitment: After IRB approval of the study protocol and FDA IND approval of the test compound/s, patients will be selected/ recruited to the study based on history of stimulant use disorder or suspected and with objective evidence of stimulant and synthetic (fentanyl) opioid exposure and use. In the case of any subjects having stimulant and synthetic opioid overdose, they will be treated with the current standard of care for opioid overdose reversal, the mu opioid receptor antagonist, naloxone. The dose of drug used for the trial will be in a range and/or combination that has been demonstrated to have a minimal side effect profile in adult humans as per existing and IND human safety study data.

Trial B: Population and setting: The study trial will involve adult patients 18-50 of individuals with stimulant use disorder suspected of and with objective evidence of stimulant and synthetic (fentanyl) opioid exposure and use. The trial will be conducted in several urban- out of hospital settings of high endemic areas for stimulant and synthetic opioid (fentanyl/F/FA) overdose. Studies will be based in large urban areas where F/FAs represent a significant proportion (>60%) of all opioid and stimulant drug overdoses (e.g. Boston, Miami, Cincinnati, Buffalo).

Trial B: AIM 3 Data collection/Data Sources: Forms will be designed to confidentially keep track of all records and exam results with all records and data stored in a HIPAA compliant fashion. An extensive data encryption plan will be reviewed and approved by IRB and IT committees of participating hospitals or prior to implementation of the study or the collection of patient data.

Trial B: Study Measures: The physical signs and symptoms associated with acute stimulant and synthetic (fentanyl) opioid overdose and morphine derived alkaloids (respiratory depression-RD) will be taught and explained to all participants and testing of concepts will be performed with grading criteria to assess objective material retention. Continued participation will be partly contingent on scoring of material retention.

Primary Outcome: Is survival rate and number of episodes of overdose compared over 6 month trial period with individuals receiving placebo and will be statistically analyzed for number of contaminated specimens. Optimal statistical analysis will be determined based on the data quality and consultation with a biostatistician. Secondary Outcome: Number of episodes of overdose compared over 6 month trial period with individuals receiving placebo and will be statistically analyzed for number of contaminated specimens. An overall goal of the study is identification of optimal lead compound efficacy in humans for treating prophylactically the physical signs and symptoms associated with acute stimulant and synthetic (fentanyl) opioid overdose and reversal of F/FA overdose compared to naloxone and standard therapies and routine pharmacological support for hypertensive crisis and/or a cardiovascular event and/or a CNS event such as seizure or stroke and a clinical presentation indicative of fentanyl or fentanyl analogues related overdose (e.g. rapid loss of consciousness after injection, rapid onset of cyanosis, chest and upper body rigidity, multiple doses of naloxone used and little or no response, needles and tourniquet still found in/on arm, sudden onset of rigidity or “seizure-like” activity after injection etc.) and a stimulant overdose (e.g. severe hypertension, seizure, evidence of a neurologic event such as stroke or a myocardial event with ischemia or an arrhythmia).

Trial B: Intervention Power Analysis: Data will be analysis and Power calculations will be performed on consultation with statistical analyst.

Based on these preliminary calculations and analysis adapted from comparable studies assessing prophylaxis agents from prior clinical studies of naloxone and naltrexone in the opioid overdose (as adapted from Parmar et al., Addiction. 112(3):502-515, 2016; Murphy et al., Addiction. 112(8):1440-1450, 2017; and Larochelle et al., Ann Intern Med. 169(3):137-145, 2018), we will assess these compounds for their effectiveness as prophylaxis agents.

Trial B: Expected Results: “Naltrexone (+)” or Nalmefene (+) are expected to increase survival rates, decrease severity and lethality of effects and decrease incidence rates of overdose from stimulants combined with synthetic opioids of the fentanyl class and identify compounds that may increase survival rates. WCS appears to be the key cause of rapid death and escalating numbers of death in the current F/FA driven opioid crisis, however, individuals who suffer from stimulant and polysubstance abuse that combines the synthetic opioid fentanyl or a comparable analogue intentionally or unintentionally with stimulants (e.g. methamphetamine, cocaine), they appear to have increased mortality compared with either agent alone. The lethal effects of either drug appear to be augmented by modulation of norepinephrine levels by each drug and directly relate to the underlying pharmacologic mechanisms whereby each drug has lethal effects on vascular and respiratory systems. We anticipate that patients receiving “Naltrexone (+)” or Nalmefene (+) will be less likely to require intubation/ invasive airway management and multiple doses of medication before primary and secondary outcomes are achieved. We also anticipate that overall mortality and morbidity will decrease and the survival rate will be significantly improved for “Naltrexone (+)” or Nalmefene (+) patients who have overdosed or were exposed to stimulants combined with synthetic opioids of the fentanyl class compared with controls and placebo groups.

### Example 8

### Compositions for Opiate and Opioid Prevention and Reversal and Methods of Their Use

Fentanyl Off-Target Sites of Action /Alpha 1 Adrenergic Subtype Binding and Implications for New Therapeutic Agents for combined stimulants and fentanyl and fentanyl analogues/Strategies in the Opioid Crisis

The series of experiments described in this Example were designed to systematically examine and compare the in-vitro binding of fentanyl and morphine to human adrenergic receptors and monoamine transporters and compare the binding affinity of several FDA approved drugs/agents at the same binding sites to identify lead molecules or therapeutic agents that could potentially reverse or antagonize these fentanyl effects in-vivo and potentially block the enhancing effects of stimulants on NE release and sustained NE activity when combined with F/FAs.

The series of experiments were designed in a direct effort to gain preclinical data supporting the development of drugs that can reverse the effects of F/FAs in in FIMR and FIRE syndrome, with particular emphasis on sympathetic effects on the larynx, vocal cords and the cardiovascular system. Similarly, the data can be used for the development of drugs which can antagonize the combined deadly effects of stimulants and F/FAs. In addition, this data were designed to assist in the development of more effective and accurate animal models for FIMR and FIRE syndrome and to antagonize or inhibit the accelerated death and severity of clinical symptoms when F/FAs are combined with stimulants. It is the hope that these models can ultimately be translated to human studies and trial designs. The data in this disclosure demonstrates that F/FAs may act in several ways to increase CNS noradrenergic activity and overlap mechanistically with stimulants, including binding adrenergic receptors and monoamine transporters and compares the binding affinity of several FDA approved drugs at the same binding sites to identify lead molecules/therapeutic agents that could reverse or antagonize these effects. However, until this disclosure there is little or no information available that directly compares F/FAs and MSO₄ binding at these receptors involved in FIMR/FIRE syndrome. These data support the fundamental difference between F/FAs and MSO4 in NE modulation. Fentanyl's pharmacological profile at noradrenergic receptors and transporters seems to resemble the effects of some known pro-noradrenergic agents such as norepinephrine re-uptake inhibitors (NERUI) and may overlap with the similar and known underlying mechanisms of stimulant drugs. When this is combined with direct agonism of alpha 1 adrenergic receptors (e.g. by stimulants) and selective alpha 1 adrenergic receptor isolation to “facilitate” norepinephrine (NE) binding at alpha 1 postsynaptic excitatory receptors would be particularly lethal.

This series of assays may help to identify the different receptor binding characteristics that may cause FIMR /FIRE syndrome. In addition, I speculated that these results might make fentanyl a useful tool to more fully characterize the pharmacological profile required for FIMR /FIRE syndrome and identify alpha adrenergic agents capable of displacing F/FA activity at these receptors and transporters. The results indicate that fentanyl and carfentanil not morphine has affinity for specific alpha 1 adrenergic receptors affinity. Thus, fentanyl and carfentanil, but not morphine displays pharmacologic effects and binding affinity along with other noradrenergic receptor binding agents such as alpha 1 adrenergic antagonists and agonists and the endogenous catecholamines norepinephrine. Additionally, stimulants (methamphetamine) and fentanyl bind to recombinant human VMAT2 transporters which can increase synaptic NE availability and stimulate specific aspects of noradrenergic action although activity of these agents were not directly reported here in this series.

FIRE Objectives: Using radioligand binding and assays of function, the interaction of fentanyl (F), carfentanil (CF), morphine (MSO₄), naloxone (NX), norepinephrine (NE), prazosin and tamsulosin were examined with recombinant human neurotransmitter receptors (e.g. adrenergic receptors) and transporters (e.g. DAT, NET, VMAT2).

Materials: Racemic fentanyl, carfentanil, naloxone, morphine, norepinephrine, prazosin and tamsulosin were obtained from Perkin Elmer Life and Analytical Sciences (Boston, MA), Sigma and Fisher Scientific chemical distributors. Structures and purity were independently verified in the laboratories used for service [³H]norepinephrine, fentanyl, carfentanil, morphine, naloxone, prazosin, tamsulosin and [³H]7-chloro-3-methyl-1-phenyl-1,2,4,5-tetrahydro-3-benzazepin-8-ol (SCH23390), [^{3H}]N-(1-benzyl-2-methylpyrrolidin-3-yl)-5-chloro-2-methoxy-4-(methylamino) benzamide (YM-09151-2, nemonapride), [¹²⁵l]methyl (1 R,2S,3S)-3-(4-iodophenyl)-8-methyl-8- azabicyclo[3.2.1]octane-2-carboxylate) (RTI-55), were purchased from Perkin Elmer Life and Analytical Sciences (Boston, Mass.). Commonly used reagents were obtained from commercial sources except where specified below.

Objectives: Using radioligand binding and assays of function, we examined the interaction of fentanyl (F), carfentanil (CF), morphine (M504), naloxone (NX), norepinephrine (NE), prazosin and tamsulosin with recombinant human neurotransmitter receptors (e.g. adrenergic receptors) and transporters (e.g. DAT, NET, VMAT2).

Tissue Culture. Human embryonic kidney cells (HEK-293) were cultured and transfected with the respective recombinant human receptor or transporter using modifications of previously described methods (Eshleman et al., 1999, Eshleman et al., 2013).

Receptor binding assays. Radioligand binding experiments were conducted by modifications of previously described methods (Eshleman et al., 1999; Gatch et al., 2011, Eshleman et al., 2013) using validated receptor and transporter characterization panels.

Recombinant Human Transporter Binding and neurotransmitter uptake assays. HEK cells expressing the recombinant human dopamine transporter (HEK-hDAT), SERT (HEK- hSERT) or norepinephrine transporter (HEK-hNET) were used as described previously (Eshleman et al., 1999). Assays were conducted as described previously (Gatch et al., 2011, Eshleman et al., 2013).

Data analysis. For radioligand binding, data were normalized to the binding in the absence of a competitive (naloxone, fentanyl, etc.) drug. Three or more independent competition experiments were conducted with duplicate determinations. GraphPAD Prism was used to analyze the subsequent data, with IC₅₀ values converted to Kᵢ values using the equation (Kᵢ=IC₅₀(1+([drug*]/Kd drug*))), where [drug*] is the concentration of the labeled ligand used in the binding assays (Cheng & Prusoff, 1973). The Kd values used in the equations are listed in Eshleman et al. (2013). Differences in affinities were assessed by one-way ANOVA using the logarithms of the Ki values for test compounds. Tukey's multiple comparison test was used to compare the potencies and efficacies of test compounds. For functional assays, GraphPAD Prism is used to calculate either EC₅₀ (agonists) or IC₅₀ (antagonists) values using data expressed as % 5HT-stimulation for IP-1 formation and % quinpirole-stimulation for mitogenesis assays. For functional assays, one-way ANOVA was used to assess differences in efficacies using normalized maximal stimulation, and differences in potencies using the logarithms of the EC₅₀ values for test compounds. Tukey's multiple comparison test was used to compare test compounds with significance set at p<0.05.

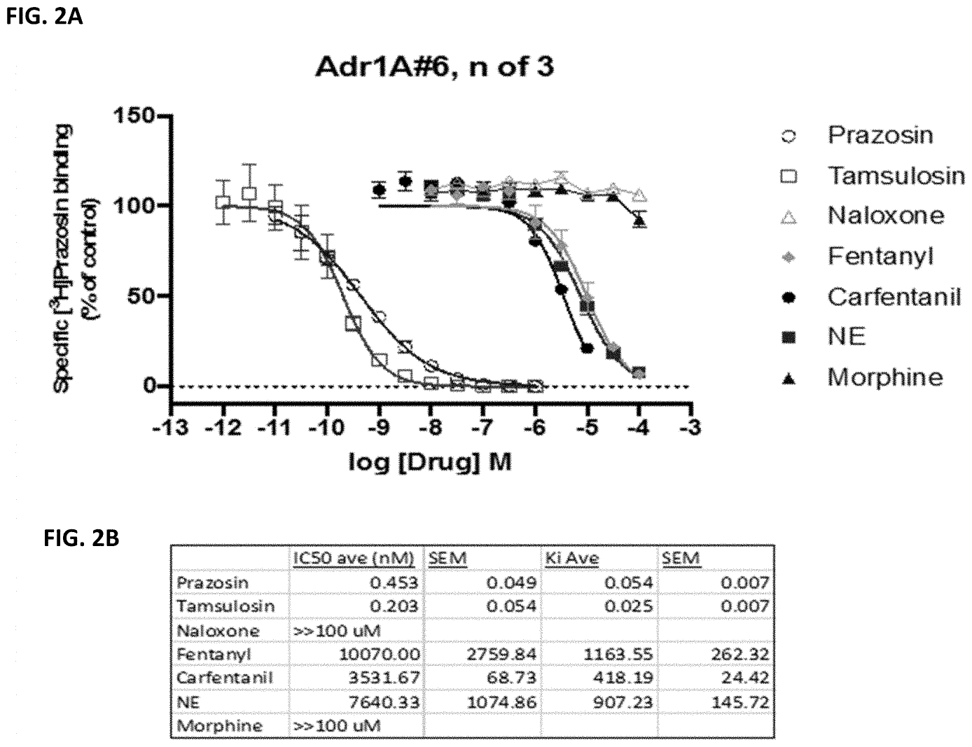

RESULTS: Summary of fentanyl, carfentanil, naloxone, norepinephrine, prazosin, tamsulosin and morphine interactions with adrenergic receptors and VMAT2. ADRENERGIC RECEPTORS: Alpha 1 adrenergic receptors (ADR1A, ADR1B, ADR1D) were combined with: [³H]Prazosin, [³H] tamsulosin, [³H]NE, [³H]fentanyl, [³H] carfentanil, [³H]morphine and [³H]naloxone to examine binding interactions to Adrenergic 1A, 1B and 1D receptors. ALPHA 1A, 1B & 1D adrenoceptors binding affinity comparison to Prazosin, Tamsulosin, Naloxone, Fentanyl, Carfentanil, NE and Morphine are shown in FIGS. 2A-2B, 3A-3B, and 4A-4B.

FIG. 2A-2B, Adr1A: fentanyl and carfentanil, but not morphine or naloxone bind all alpha 1 subtypes (e.g. 1A, 1B, 1D). At the alpha 1A receptor as shown in FIG. 2A and FIG. 2B, fentanyl has comparable affinity, as seen by Ki values, as NE. Carfentanil, in contrast has a 2 fold greater affinity at the 1A compared to fentanyl and NE. By comparison, prazosin and tamsulosin each have BA in the subnanomolar (<1 nM) range at all subtypes and BA that is 4-5 orders of magnitude greater than fentanyl, carfentanil or NE. Additionally, prazosin and tamsulosin have a 4-6 orders of magnitude greater BA at each subtype over either fentanyl or NE.

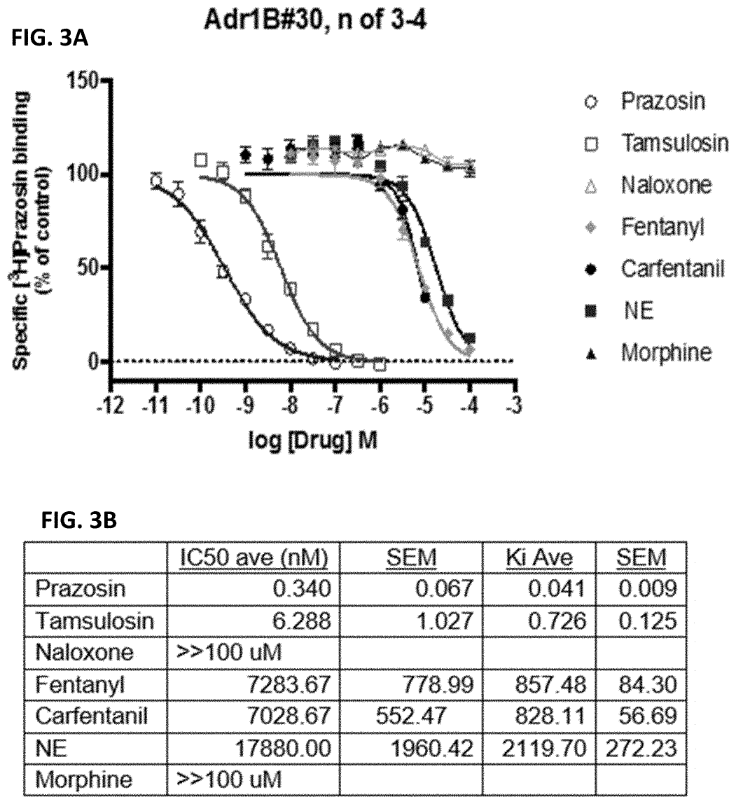

FIG. 3A-3B, Adr1B: Fentanyl and carfentanil, but not morphine or naloxone bind all alpha 1 subtypes (e.g. 1A, 1B, 1D). At the alpha 1B receptor as shown in FIG. 3A and FIG. 3B, fentanyl has comparable affinity as carfentanil, as seen by Ki values, and in contrast has a 2 fold greater affinity at the 1B compared to NE. By comparison prazosin and tamsulosin each have BA in the subnanomolar (<1 nM) range at all subtypes and BA that is 4-5 orders of magnitude greater than either fentanyl, carfentanil or NE. Additionally, prazosin and tamsulosin have a 4-5 orders of magnitude greater BA at each subtype over either fentanyl, carfentanil or NE.

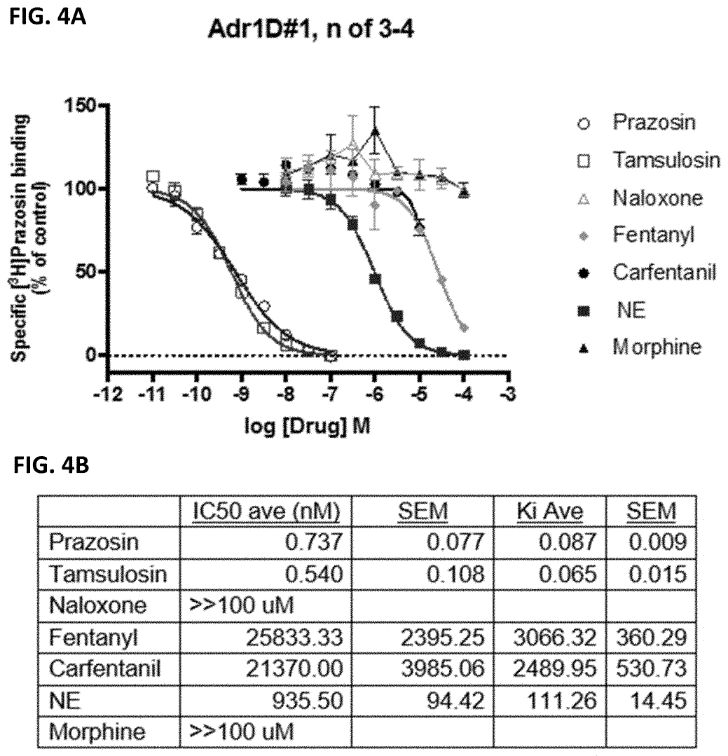

FIG. 4A-4D, Adr1D: Fentanyl and carfentanil, but not morphine or naloxone bind all alpha 1 subtypes (e.g. 1A, 1B, 1D). At the alpha 1D receptor as shown in FIG. 4A and FIG. 4B, fentanyl and carfentanil have comparable affinity, as seen by Ki values. NE, in contrast has a ˜25-30 fold greater affinity at the 1D compared to carfentanil and fentanyl, respectively. Notably, the 1D subtype is where NE demonstrates its greatest binding affinity. By comparison prazosin and tamsulosin each have BA in the subnanomolar (<1 nM) range at all subtypes and BA that is 4-6 orders of magnitude greater than either fentanyl, carfentanil or NE. Additionally, prazosin and tamsulosin have a 4-6 orders of magnitude greater BA at each subtype over either fentanyl, carfentanil or NE.

Discussion:1. Fentanyl (F), carfentanil (CF), norepinephrine (NE), epinephrine (EPI), prazosin and tamsulosin all bound to alpha 1 adrenergic receptor subtypes with varying affinity (Kᵢ=0.025 nM-3066 nM). Of notable exception, neither morphine (MSO₄) or naloxone had any notable or relevant binding activity at any of the receptors or transporter in the series. However, fentanyl (F) and carfentanil (CF) but not morphine or naloxone, demonstrate binding at all alpha 1 subtypes. F and CF demonstrate greater binding affinity (BA) than NE in the case of the 1A and 1B subtypes, but showed ˜25-30 fold less binding affinity (BA) at the 1D subtype, where NE demonstrates its greatest binding affinity. Although NE is a well-known alpha 1 adrenergic agonist, its subtype specificities and binding affinity (BA) values at human alpha 1 adrenoceptors have not been previously demonstrated in published literature, but we demonstrated these quantitative values for NE and additionally demonstrated that NE has variable binding affinity at alpha 1 subtypes with binding at the 1D subtype that is ˜9-20 fold over 1 A and 1B receptor subtypes, respectively. This creates a plausible mechanism whereby either fentanyl or carfentanil can competitively occupy (e.g. antagonize) alpha 1A and 1B receptor subtypes with greater affinity than NE and make more NE available to agonize the 1D subtype where NE has its greatest BA by 9-20 fold over 1A and 1B subtypes and where it has superior affinity over fentanyl and carfentanil by ˜25-30 fold. The ultimate effect is for these F/FAs to isolate and increase the availability of NE to agonize the 1D subtype with presumably an enhanced and specific physiologic response. Considering the fact that each alpha 1 subtype has a specific and dominant distribution pattern in various key components of the vascular system (e.g. myocardium, Right ventricle, Left ventricle, venous system, arterial system, pulmonary arterial system and coronary arteries), the ability of F/FAs to selectively isolate subtypes and concentrate NE activity at a particular subtype, has significant implications for the isolation and derangement of the normal alpha 1 subtype binding in these various system components with the potential for asymmetric and/or extreme or aberrant physiologic reactions. These NE driven mechanisms have particular relevance in the context of their recent use in combination with stimulants such as fully synthetic methamphetamines and natural alkaloids such as cocaine, which actively enhance NE release and interfere with NE degradation. The presence of increased NE in the presence of F/FAs has the potential to accelerate the underlying noradrenergically driven mechanisms of FIRE syndrome.

In addition, recent data indicates that fentanyl and carfentanil, but not morphine, binds the VMAT 2 transporter in a pattern consistent with that of molecules that may act as reuptake inhibitors (e.g. methamphetamine) and offers further support for the enhanced function of noradrenergic activity by F/FAs and the increased lethality of either F/FAs and stimulants when the two drug classes are combined. However, this is particularly relevant in the context of the effects of stimulants (e.g. methamphetamine and cocaine) which can augment release of NE in the CNS by these same transporters, prevent breakdown or reuptake of NE and increase the amount of NE available to bind and agonize the post-synaptic and excitatory alpha 1 adrenoreceptors as the key component of the underlying noradrenergically driven mechanisms of FIRE syndrome.

Conclusions: Fentanyl and carfentanil, but not morphine, act as antagonists at all alpha 1 subtypes (e.g. 1 A, 1 B, 1 D) and have greater BA than NE (e.g. the endogenous ligand) except at 1 D, where NE has 30X greater BA than fentanyl and ˜20X greater than carfentanil. By comparison prazosin and tamsulosin each have BA in the subnanomolar (<1 nM) range at all subtypes and BA that is 4-5 orders of magnitude greater than fentanyl, particularly at the 1D subtype. Both prazosin and tamsulosin act as antagonists with approximately 25-35,000 X greater potency by IP1 assay than fentanyl or carfentanil at the 1 D.

In addition, fentanyl and carfentanil, but not morphine, binds VMAT2 transporter in a pattern consistent with that of molecules that may act as reuptake inhibitors (e.g. methamphetamine) and offers further support for the enhanced function of noradrenergic activity by F/FAs and the increased lethality of either F/FAs and stimulants when the two drug classes are combined. When looked at together, the possibility of NE reuptake inhibition by fentanyl and carfentanil by various mechanisms, combined with fentanyl's isolation of the 1D subtype for concentrated NE activation provides a plausible underlying mechanism for increased noradrenergic signaling as an underlying mechanism in FIRE syndrome and offers a significant interventional target of alpha 1 adrenoceptors at postsynaptic terminals. Although the VMAT2 is mentioned as an interesting and supportive mechanism it does not provide a clear target for interventional therapy and is not mentioned for such purposes as an invention or part of this current invention. These findings are particularly significant, given the current high incidence of overdose deaths from F/FAs and particularly when combined with stimulants. The current data also suggest the underlying mechanisms for FIRE syndrome and strategic interventional targets that may more effectively improve survival from combined F/FAs and stimulant overdose. In conclusion, these effects may be relevant in the development of therapeutics that target the underlying mechanism of the adverse effects (e.g. vocal cord and upper airway effects) and deaths associated with FIRE syndrome. Additionally, these targets are of even greater relevance when F/FAs are combined with stimulants which enhance the release of NE and can clinically accelerate and/ or increase the consistency of life threatening respiratory and cardiovascular effects of F/FAs and may explain the accelerated and/or consistent lethality that has been reported when these two drug classes are combined.

Additionally, characterizing interactions with this panel of receptors and transporters may be useful for characterizing effects of other drugs or molecules that may target the underlying mechanism of FIRE syndrome and to screen new opioids for similar binding patterns to avoid similar issues with muscle rigidity and airway compromise as seen with F/FAs. The assays used for proof of concept here offer a consistent set of analytical tools and assay sets to assess the underlying mechanisms of other more potent and illicit synthetic opioids/fentanyl analogues as they emerge in the ongoing opioid F/FA driven crisis.

### REFERENCES:

Aghajanian, J Clin Psychiatry. 43(6 Pt 2):20-24, 1982
Baumann et al., Trends in Pharmacological Sciences 39:995-998, 2018
Bennett et al., Anesthesiology 87:1070-1074, 1997
Burns et al., Clinical toxicology (Philadelphia, Pa.) 54:420-423, 2016
Cheng & Prusoff, Biochem. Pharmacol., 22, 3099-3108, 1973
Clarke et al., Emergency medicine journal: EMJ 22:612-616, 2005a
Eshleman et al., J Pharmacol Exp Ther. 289(2):877-85, 1999
Eshleman et al., Biochem. Pharmacol. 85(12):1803-15, 2013
Fu et al., Neuroscience Letters 165:199-202, 1994
Gatch et al., J Pharmacol Exp Ther. 338(1):280-9, 2011
Gazi et al., Br J Pharmacol 128(3):613-20, 1999
Gillespie et al., Bioorg Med Chem Lett. 18(9):2916-9, 2008
Glimcher, Proc Natl Acad Sci U S A. 108 Suppl 3:15647-54, 2011
Grell et al., Anesthesia and Analgesia 49:523-532, 1970
Janssen Pharmaceutica, Sublimaze Injection (fentanyl tcitrate) [package insert]. Janssen Pharmaceuticals, Inc., Beerse, Belgium, 2017
Kanagarajadurai et al., Mol Biosyst. 5(12):1877-88, 2009
Kelly et al., Nature. 459(7244):270-3, 2009
Knight et al., Naunyn-Schmeideberg's Arch Pharmacol 370:114-123, 2004
Korf et al., Eur J Pharmacol. 25(2):165-169, 1974
Lalley, Am J Physiol Regul Integr Comp Physiol. 285(6):R1287-304. Epub 2003
Lui et al., Neuroscience Letters 96:114-119, 1989
Lui et al., Neuroscience Letters 108:183-188, 1990
Lui et al., Neuroscience Letters 157:145-148, 1993
Lui et al., Neuroscience Letters 201:167-170, 1995
McClain & Hug, Clinical pharmacology and therapeutics 28:106-114, 1980
Minuzzi & Cumming, Neurochem Int 56(6-7):747-752, 2010
Neve et al., Molecular biology of dopamine receptors. In: The Dopamine Receptors. Neve KA and Neve RL, Eds. Humana Press, Totawa NJ, 1997
O'Donnell et al., MMWR Morbidity and mortality weekly report 66:1197-1202, 2017
Rudd et al., MMWR Morbidity and mortality weekly report 65:1445-1452, 2016
Sarihi et al., J Neurosci. 32(38):13189-99, 2012
Scamman, Anesthesia and Analgesia 62:332-334, 1983
Slavova et al., Pharmaceutical Medicine 31:155-165, 2017a
Slavova et al., The International journal on drug policy 46:120-129, 2017b
Sleight et al., Biochem Pharmacol 51(1):71-6, 1996
Somerville et al., MMWR Morbidity and mortality weekly report 66:382-386, 2017
Stanley, The History of Opioid Use in Anesthetic Delivery, in The Wondrous Story of Anesthesia (Eger li El, Saidman LJ and Westhorpe RN eds) pp 641-659, Springer New York, New York, N.Y., 2014
Stoeckel et al., British Journal of Anaesthesia 51:741-745, 1979
Stone & Difazio Anesthesia and Analgesia 67:663-666, 1988
Streisand et al., Anesthesiology 78:629-634, 1993
Torralva & Janowksy, J Pharmacol Exp Ther. 371(2):453-475, 2019

### Example 9

### VMAT/Fentanyl binding and altered NE release:

Drugs can differ significantly between their affinities for radioligand binding sites on neurotransmitter transporters and their potencies at inhibiting transporter function. In the case of norepinephrine transporters (e.g. NET and VMAT), inhibition of these transporters in presynaptic terminals makes norepinephrine more available for release from these terminals in the case of presynaptic activation. Methamphetamine and cocaine are two common stimulants whose pharmacologic actions are specifically related to their ability to inhibit or modify NET and VMAT function, thus increasing the availability of catecholamines for neurotransmission which relates directly to the euphoria and stimulant effects associated with these drugs.

Preliminary data on fentanyl's interaction with recombinant human transporters and the vesicular monoamine transporter—VMAT demonstrates that fentanyl (and not morphine) binds to the VMAT and blocks uptake of neurotransmitter with significantly greater potency than methamphetamine (e.g. half maximal inhibitory concentration-IC₅₀ value of 911 nM vs ˜4000 nM for methamphetamine) and along with methamphetamine, binds to a site on the VMAT that appears to be more closely related to transporter function than the well-defined VMAT radioligand [³H]dihydrotetrabenazine, [³H]DHTB (Eshleman et al., Biochem Pharmacol 85:1803-1815, 2013; Provencher et al., J Med Chem 61:9121-9131, 2018).

Taken in the context that the availability and release of norepinephrine are the neurophysiological pre-requisites for activation of locus coeruleus-cerulopsinal motor fibers that cause rigidity in respiratory muscles of the chest wall and for the activation of cerulomedullary fibers which cause disruptions of vagally mediated vocal cord patency, an increase in availability of norepinephrine by high dose fentanyl (F/FAs) would explain the physiologic manifestations seen clinically with FIRE syndrome. Furthermore, it emphasizes the significant danger and lethality risk when F/FAs are combined with stimulants which alter norepinephrine and catecholamine levels as a primary mechanism of action by their pharmacological class.

In either case, when considered with the selective alpha 1 adrenoceptor subtype binding at post synaptic terminals seen with F/FAs, the increase in norepinephrine availability for binding the 1D subtype is fundamental to the underlying mechanism of FIRE syndrome and offers a clear interventional target for which therapies are described in detail in this patent disclosure. These data, along with other previously published results, suggest that there are significant differences between morphine and fentanyl pharmacology indicating that the effects of F/FAs on animal models of FIMR and FIRE can be blocked with non-MOR antagonists and point to new directions for the development of treatments for the effects of synthetic opioids (F/FAs).

### Example 10

### Development and Use of a Rat Airway Monitoring Model

Using methods largely as described in Example 6, this Example details development of a rat airway monitoring model for lead compound identification for F/FA exposure, and provides an illustrative use.

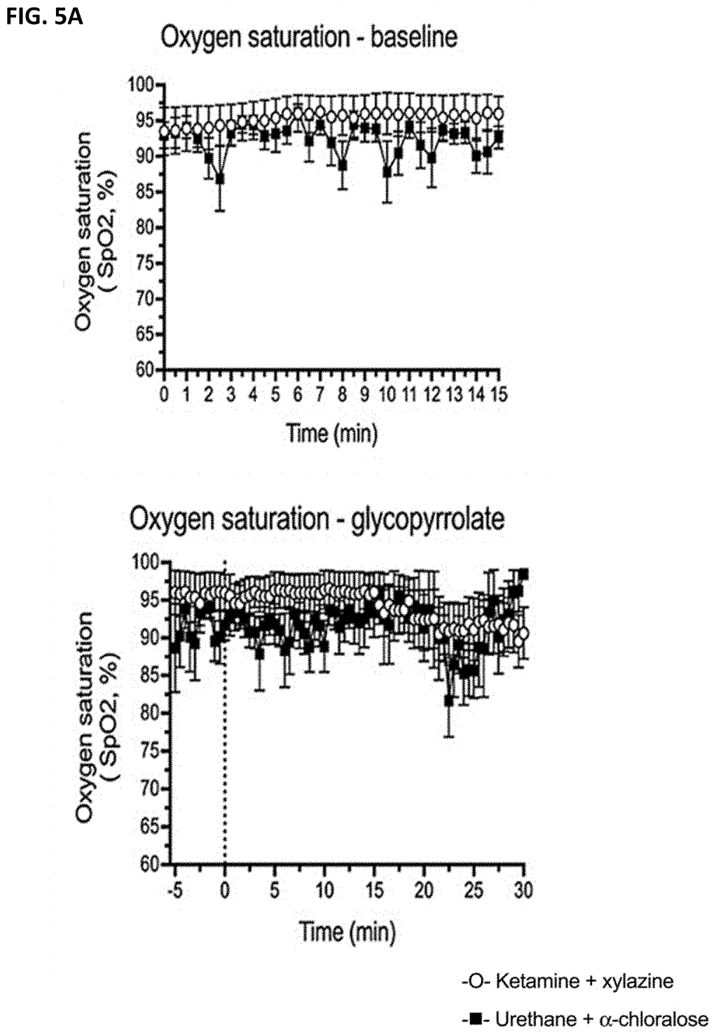

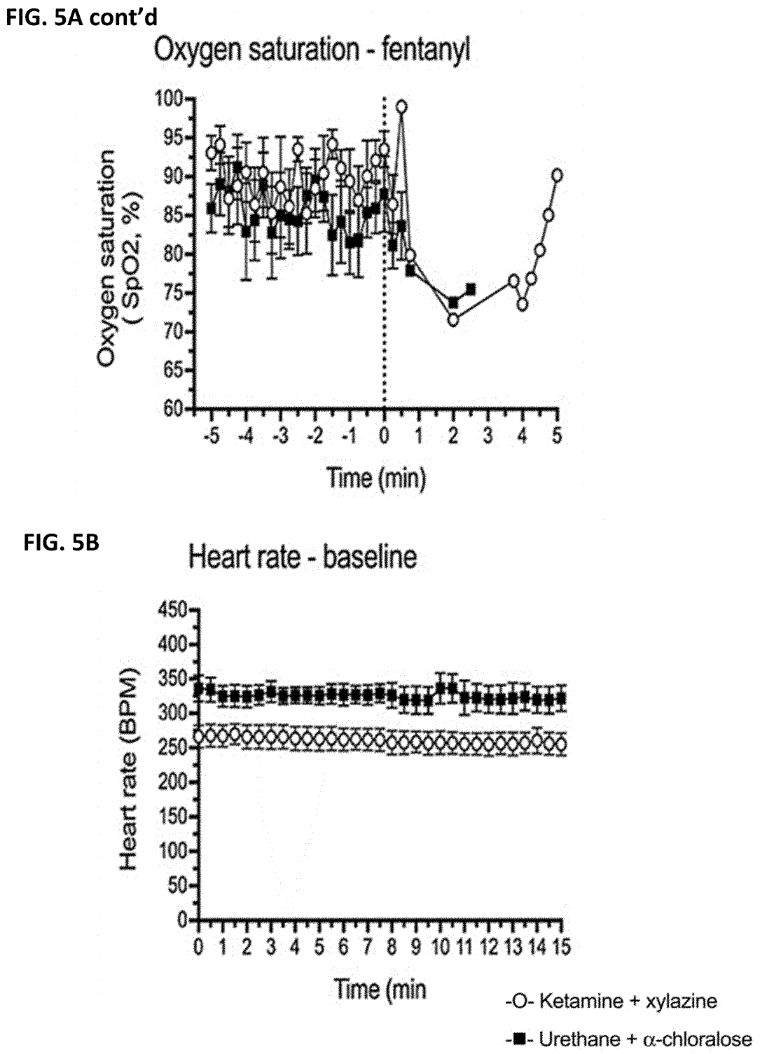

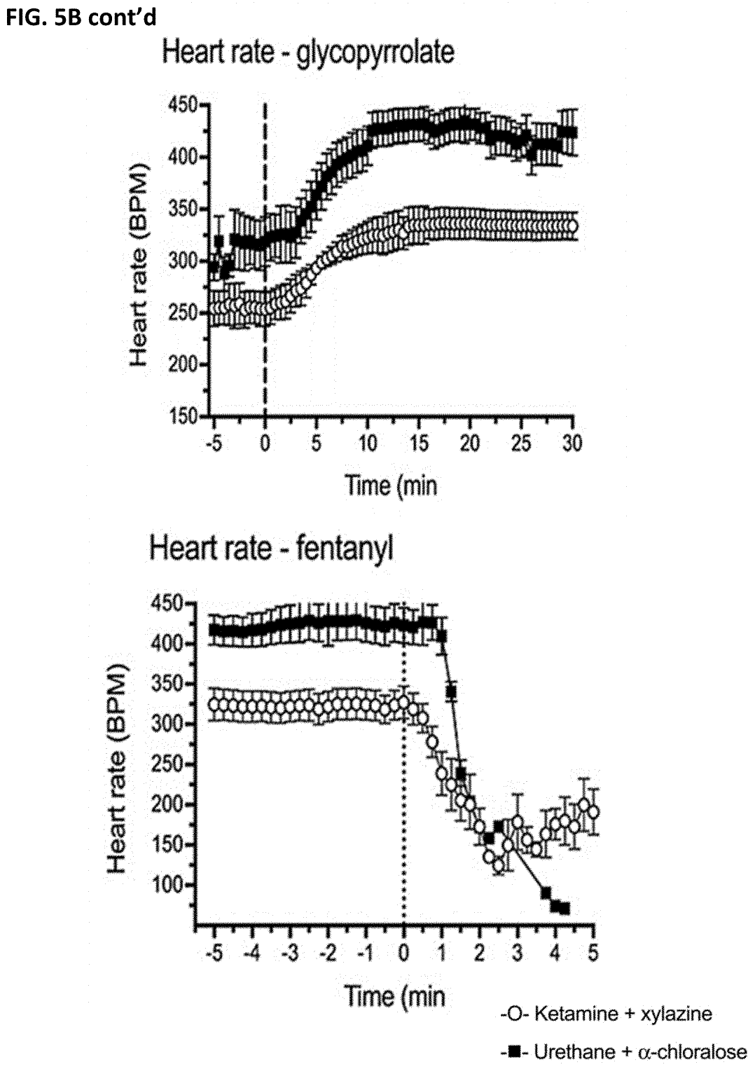

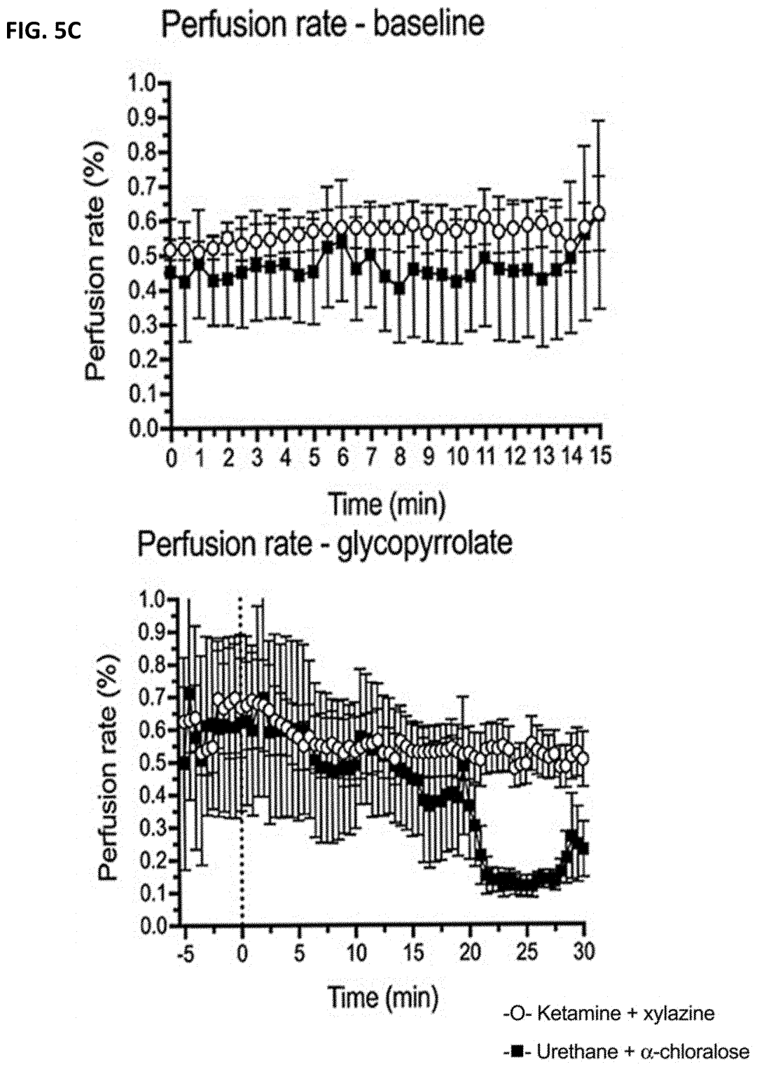

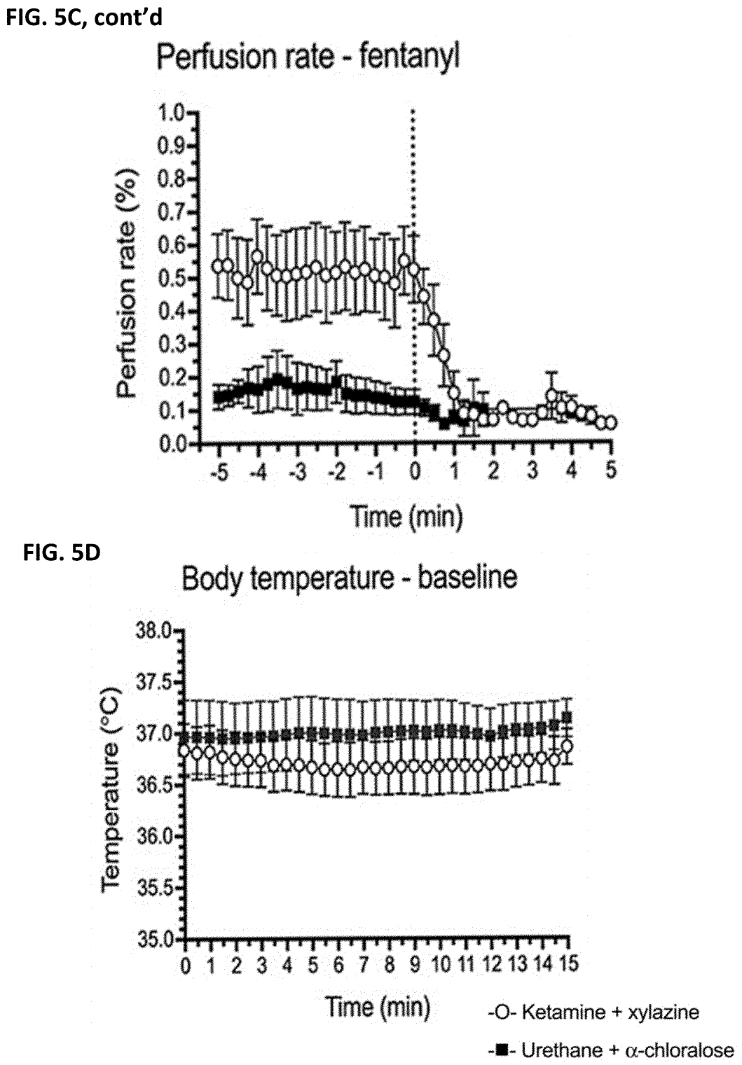

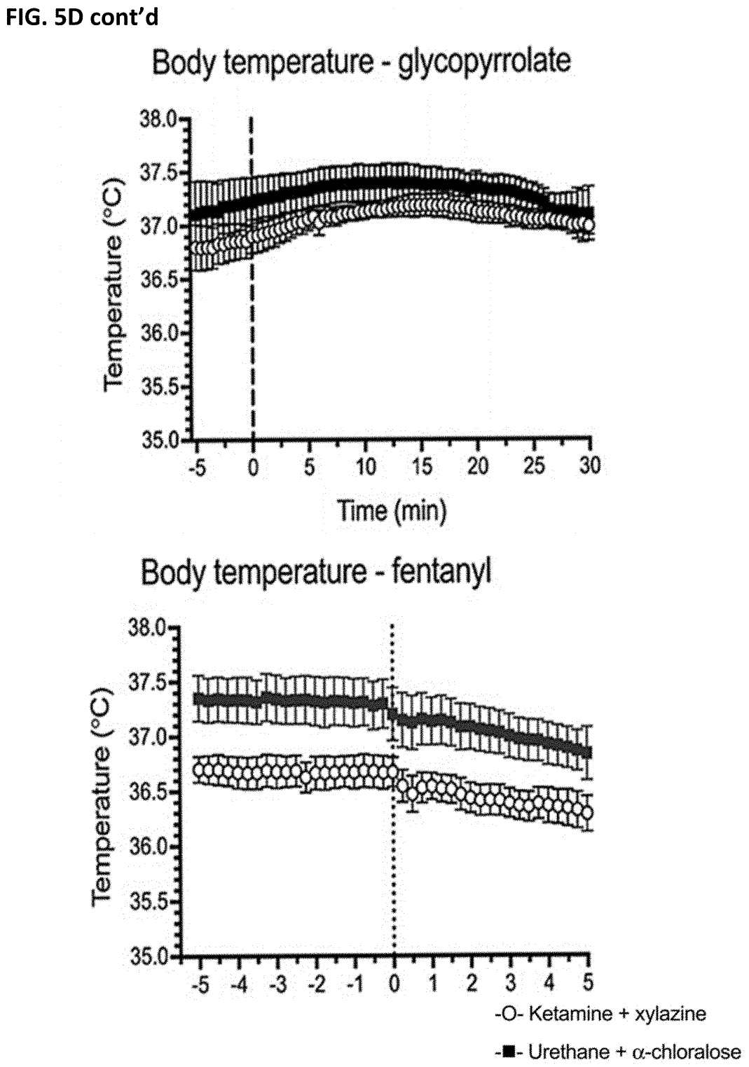

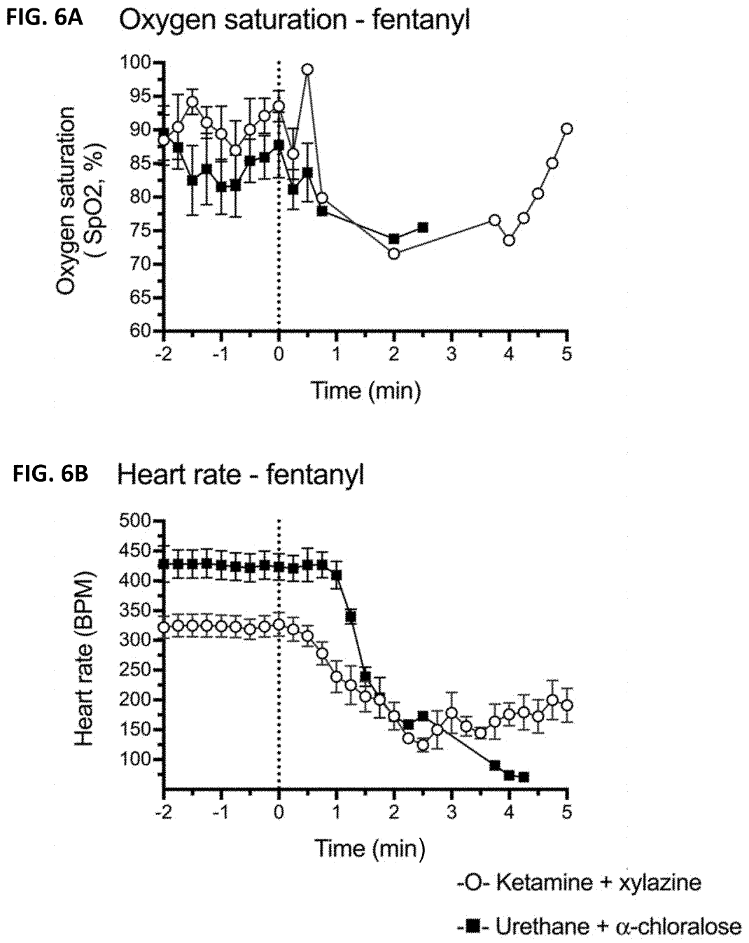

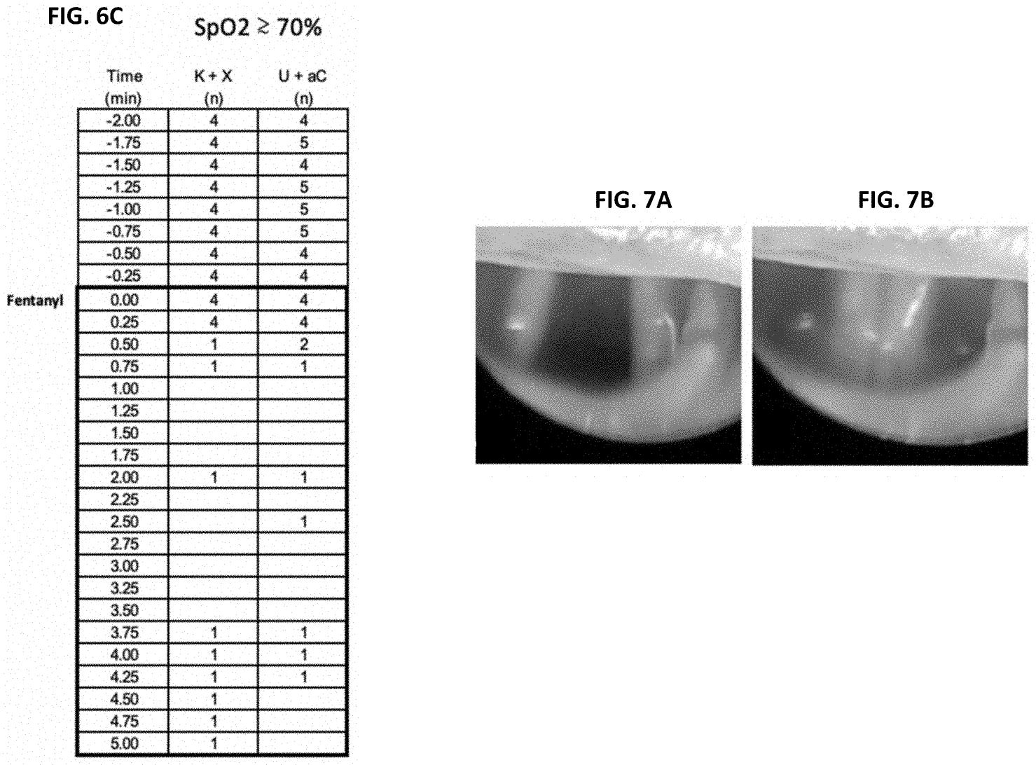

On the day of the procedure, rats (male and female Sprague Dawley, 250-300 gm) were administered ketamine (e.g. 80 mg/kg and xylazine 8 mg/kg, i.p.). Alternatively a dose of urethane 0.9-1.8 mg/kg and alpha-chloralose 40 mg/kg via intraperitoneal injection were administered as an alternate anesthetic agent as it is significantly longer in duration for circumstances when longer experimental observation is required, has no alpha 1 adrenergic receptor activity and minimal effects on airway secretions and upper airway visibility. Supplemental glycopyrrolate 0.5 mg/kg is administered 30 minutes prior to airway instrumentation and is used as an antisialagogue to minimize airway secretions and maximize airway and vocal visibility. After onset of surgical anesthesia verified by lack of response to 2 second paw pinch, animals were immobilized on a rodent intubating stand or supine on a heated surgical table. Eyes were lubed with Lacri-Lube® eye gel and a rectal temperature probe was placed prior to surgical vascular access procedures. PhysioSuite monitors were placed on a paw for pulse oximetry oxygen saturation measurement, perfusion rate and heart rate. The temperature probe was also monitored by the physio-suite device. See FIGS. 5A-5D for representative results over time during this experiment. Additional measurements are shown in FIGS. 6A-6C.

The skin of the lower abdomen was then prepared by removing hair with an electric razor, and skin was then prepared in sterile fashion with alcohol swabs and povidone iodine swabs. A lower abdominal wall incision was made at the level of the inguinal ligament to expose the femoral artery and femoral vein. Each vessel was cannulated with sterile surgical tubing for arterial pressure monitoring from the femoral artery and vascular intravenous injection access for the femoral vein. An oral retractor was placed to displace the tongue from the airway and a 1 ml syringe barrel was placed midline in the oropharynx as an introducer guide for the 2.7 mm rigid endoscope to visualize epiglottis and vocal cords prior to injection of fentanyl. Once vocal cords were visualized, the video camera attached to the endoscope was activated to begin recording video images in real time prior to fentanyl injection and after injection for up to 10 minutes if the animal continues to demonstrate open vocal cords, persistent heart rate, oxygen saturation and respiratory rate.

Oxygenation was measured using pulse oximetry, and respiratory rate as measured by precordial chest auscultation of breath sounds with output to an audio recorder with a visual display. Cardiac function is measured using heart rate and hemodynamics are measured continuously with invasive arterial catheter monitoring. The femoral artery and vein were cannulated and can be used for blood samples, arterial pressure monitoring, and drug administration. Rectal temp will be kept at 37+/-0.5²C using a heat lamp and temperature controller. Adequate general anesthesia and analgesia were maintained to allow for invasive procedures, but to maintain spontaneous respiration to facilitate vocal cord visualization. The video endoscope was positioned for continuous visualization of the larynx. (See FIGS. 7A and 7B for rodent vocal cord closure pre (FIG. 7A) and post (FIG. 7B) intravenous fentanyl bolus).

It has been demonstrated using the described experimental model that VCC along with chest wall and limb rigidity is a prominent feature in the animal model when high dose F/FAs were administered. In a series of 8 animals administered fentanyl 100 μg/kg IV bolus over 10 seconds, 8/8 animals developed vocal cord closure and muscle rigidity within 15-30 seconds of IV bolus. The VCC was sustained in all cases for ˜90 seconds and followed almost immediately by cardiac asystole with arterial pressure no longer detectable in each case. This pilot experiment did not include administration of a stimulant only to establish the consistency of effects from F/FA prior to potentially accelerating the reaction with the addition of stimulants. All therapeutic agents as noted will be trialed under conditions that combine both F/FA and stimulants at various levels of toxicity.

The inventor has demonstrated in an animal model that vocal cord closure and chest wall rigidity occur simultaneously after high dose fentanyl (50-100 mcg/kg) within 15-30 seconds after intravenous bolus, persist for ˜90 seconds, whereupon the heart becomes asystolic and arterial pressure falls to 0 (zero) mm Hg and the animal cannot be resuscitated without the administration of therapeutic agents. All respiratory effort ceases at the time onset of vocal cord closure (e.g. 15-30 seconds after IV bolus). This effect is specific to F/FA and is not demonstrated with morphine, heroin or stimulants (e.g., cocaine, methamphetamine).

### (X) CLOSING PARAGRAPHS

As will be understood by one of ordinary skill in the art, each embodiment disclosed herein can comprise, consist essentially of, or consist of its particular stated element, step, ingredient, or component. As used herein, the transition term “comprise” or “comprises” means having, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. As used herein, a material effect would cause a measurable reduction in one or more symptoms of stimulant usage or overdose combined with opioid/opiate usage or overdose (for instance, reduction in high blood pressure and/or rapid heart rate or cardiac arrhythmia, chest wall rigidity, increased level of consciousness, return of spontaneous respiration and adequate tidal volumes to maintain O₂Saturations >94% by pulse oximetry) within one minute to ten minutes following administration of a disclosed combination therapy to a subject (in the case of immediate care/reversal embodiments); or a material effect would prevent or reduce the development of one or more such symptoms upon exposure to an opioid/opiate, in the case of a prophylactic embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

I claim:

1. A method of preventing or reversing one or more effects of combined opioid/opiate and stimulant exposure in a subject, comprising administering to the subject in need of such treatment:
a therapeutically effective amount of at least one α1 adrenergic receptor antagonist, and
a therapeutically effective amount of a mu receptor antagonist;
or
a therapeutically effective amount of at least one α2 adrenergic receptor agonist, and
a therapeutically effective amount of a mu receptor antagonist.

2. The method of claim 1, wherein at least one α1 adrenergic receptor antagonist targets α1-adrenergic receptor subtype 1 D.

3. The method of claim 1, wherein at least one α1 adrenergic receptor antagonist preferentially targets al-adrenergic receptor subtype 1 D.

4. The method of claim 1, wherein the a2 adrenergic receptor agonist is clonidine.

5. The method of claim 1, wherein the stimulant comprises cocaine, amphetamine, methamphetamine, or a combination of two or more thereof.

6. The method of claim 1, wherein the opioid/opiate comprises fentanyl, alfentanil, sufentanil, remifentanil, carfentanil, oxycodone, hydrocodone, hydromorphone, oxymorphone, meperidine, tapentadol, morphine, heroin, opium, codeine, or a combination of two or more thereof.

7. The method of claim 1, further comprising administering to the subject:
a therapeutically effective amount of one or more of a cholinergic agent (muscarinic antagonist/M3 agonist and/or nicotinic agonist), a centrally-acting or peripherally acting respiratory stimulant, a GABA/benzodiazepine receptor complex antagonist, a Mu receptor or opioid receptor subtype agonist, a long-acting Mu or opioid receptor subtype antagonist, a vasoactive agents, an anticholinergic agent, a centrally-acting a adrenergic receptor antagonist combined with a peripherally acting a adrenergic receptor antagonist, a muscle paralytic, a anticonvulsant, a membrane-stabilizing agent, or a Beta Blocker.

8. The method of claim 7, wherein a pharmaceutical composition is administered to the subject, which pharmaceutical composition comprises:
(IRNM1) MU+S-A1ARA; or
(IRNM2) MU+A2ARA; or
(IRNM3) MU+NS-A1ARA; or
(IRNM4) MU+S-A1ARA+/−NS-A1ARA; or
(IRNM5) MU+S-A1ARA+/−NS-A1ARA+/−A2ARA; or
(IRNM6) MU+S-A1ARA+/−NS-A1ARA+/−AC or C; or
(IRNM7) MU+S-A1ARA+/−NS-A1ARA+/−AC or C+/−A2ARA or +/−BetaB; or
(IRMnAW1) MU+S-A1ARA+/−NS-A1ARA; or
(IRMnAW2) MU+S-A1ARA+/−NS-A1ARA+/−AC or C; or
(IRMnAW3) MU+S-A1ARA+NS-A1ARA+/−AC or C+A2ARA or +/−BetaB; or
(IRMAW1) MU+S-A1ARA+/−NS-A1ARA; or
(IRMAW2) MU+S-A1ARA+/−NS-A1ARA+/−PMR or +/−BetaB; or
(Poly1) MU+S-A1ARA+NS-A1ARA+GCA; or
(Poly2) MU+S-A1ARA+NS-A1ARA+GCA+ASMS; or
(Poly3) MU+S-A1ARA+NS-A1ARA+GCA+ASMS+PMR or +/−BetaB; or
(PASOU1) MU+S-A1ARA+NS-A1ARA; or
(PASOU2) MU+S-A1ARA+NS-A1ARA+A2ARA or +/−BetaB; or
(PFR1) MU or MUXR +S-A1ARA+/−NS-A1ARA or +/−BetaB;
wherein MU=Mu receptor antagonist, A1ARA=Alpha-1 Adrenergic receptor antagonist, A2ARA=α2-adrenergic receptor agonist, AC=Anticholinergic, Beta Blockers=BetaB, C=Cholinergic, PMR=Paralytic/Muscle relaxant, GCA=GABA Complex Antagonist, and ASMS=Anti-seizure/Membrane stabilizer, and
wherein each is provided in an amount sufficient to be therapeutically effective.

9. A method of preventing or reversing one or more opioid or opiate effects and one or more stimulant effects or interactive effects of these classes of drugs in a subject, comprising administering to the subject in need of such treatment a formulated pharmaceutical composition comprising:
(IRNM1) MU+S-A1ARA; or
(IRNM2) MU+A2ARA; or
(IRNM3) MU+NS-A1ARA; or
(IRNM4) MU+S-A1ARA+/−NS-A1ARA; or
(IRNM5) MU+S-A1ARA+/−NS-A1ARA+/−A2ARA; or
(IRNM6) MU+S-A1ARA+/−NS-A1ARA+/−AC or C; or
(IRNM7) MU+S-A1ARA+/−NS-A1ARA+/−AC or C+/−A2ARA or +/−BetaB; or
(IRMnAW1) MU+S-A1ARA+/−NS-A1ARA; or
(IRMnAW2) MU+S-A1ARA+/−NS-A1ARA+/−AC or C; or
(IRMnAW3) MU+S-A1ARA+NS-A1ARA+/−AC or C+A2ARA or +/−BetaB; or
(IRMAW1) MU+S-A1ARA+/−NS-A1ARA; or
(IRMAW2) MU+S-A1ARA+/−NS-A1ARA+/−PMR or +/−BetaB; or
(Polyl) MU+S-A1ARA+NS-A1ARA+GCA; or
(Poly2) MU+S-A1ARA+NS-A1ARA+GCA +ASMS; or
(Poly3) MU+S-A1ARA+NS-A1ARA+GCA +ASMS +PMR or +/−BetaB; or
(PASOU1) MU+S-A1ARA+NS-A1ARA; or
(PASOU2) MU+S-A1ARA+NS-A1ARA+A2ARA or +/−BetaB; or
(PFR1) MU or MUXR +S-A1ARA+/−NS-A1ARA or +/−BetaB;
wherein MU=Mu receptor antagonist, A1ARA=Alpha-1 Adrenergic receptor antagonist, A2ARA=α2-adrenergic receptor agonist, AC=Anticholinergic, BetaB=Beta Blockers, C=Cholinergic, PMR=Paralytic/Muscle relaxant, GCA=GABA Complex Antagonist, and ASMS=Anti-seizure/Membrane stabilizer, and
wherein each is provided in an amount sufficient to be therapeutically effective.

10. A method of preventing or reversing one or more effect of combined opioid/opiate and stimulant exposure or overdose in a subject, comprising administering to the subject in need of such treatment a formulated pharmaceutical composition comprising:
(IRNM1) MU+S-A1ARA; or
(IRNM2) MU+A2ARA; or
(IRNM3) MU+NS-A1ARA; or
(IRNM4) MU+S-A1ARA+/−NS-A1ARA; or
(IRNM5) MU+S-A1ARA+/−NS-A1ARA+/−A2ARA; or
(IRNM6) MU+S-A1ARA+/−NS-A1ARA+/−AC or C; or
(IRNM7) MU+S-A1ARA+/−NS-A1ARA+/−AC or C+/−A2ARA or +/−BetaB; or
(IRMnAW1) MU+S-A1ARA+/−NS-A1ARA; or
(IRMnAW2) MU+S-A1ARA+/−NS-A1ARA+/−AC or C; or
(IRMnAW3) MU+S-A1ARA+NS-A1ARA+/−AC or C+A2ARA or +/−BetaB; or
(IRMAW1) MU+S-A1ARA+/−NS-A1ARA; or
(IRMAW2) MU+S-A1ARA+/−NS-A1ARA+/−PMR or +/−BetaB; or
(Polyl) MU+S-A1ARA+NS-A1ARA+GCA; or
(Poly2) MU+S-A1ARA+NS-A1ARA+GCA +ASMS; or
(Poly3) MU+S-A1ARA+NS-A1ARA+GCA +ASMS +PMR or +/−BetaB; or
(PASOU1) MU+S-A1ARA+NS-A1ARA; or
(PASOU2) MU+S-A1ARA+NS-A1ARA+A2ARA or +/−BetaB; or
(PFR1) MU or MUXR +S-A1ARA+/−NS-A1ARA or +/−BetaB;
wherein MU=Mu receptor antagonist, A1ARA=Alpha-1 Adrenergic receptor antagonist, A2ARA=α2-adrenergic receptor agonist, AC=Anticholinergic, BetaB=Beta Blockers, C=Cholinergic, PMR=Paralytic/Muscle relaxant, GCA=GABA Complex Antagonist, and ASMS=Anti-seizure / Membrane stabilizer, and
wherein each is provided in an amount sufficient to be therapeutically effective.

11. The method of claim 10, wherein the one or more effect comprises an interactive effect of the opioid/opiate and stimulant drugs.

12. The method of claim 1, wherein the one or more effects of combined opioid/opiate and stimulant exposure in the subject comprises one or more of:
fentanyl-induced muscle rigidity (FIMR),
wooden chest syndrome (WCS),
unconsciousness,
a stimulant effect selected from cardiovascular, hemodynamic, cerebrovascular, or neurologic effects), or
an interactive effect of the opioid/opiate and stimulant drugs.

13. The method of claim 12, wherein the interactive effect of the opioid/opiate and stimulant drugs comprises Stimulant and Synthetic Opioid Induced Vascular Events (SSOIVE).

14. The method of claim 1, further comprising identifying the subject as being in need of combined opiate/opioid with stimulant overdose reversal before administering the treatment.

15. The method of claim 10, wherein the subject is a human.

16. The method of claim 1, wherein administration is by intravenous (IV), intramuscular (IM), intranasal (IN), transdermal (TD), intraosseous (10), intrathecal (IT), intraocular (IOC), oral, sublingual (SL), or transtracheal (TT) delivery to the subject.

17. The method of claim 1, wherein the administration is by injection.

18. The method of claim 1, wherein the administration is administration of a pharmaceutical composition formulated to be delivered to the subject as a premeasured single dose.

19. The method of claim 1, wherein the Mu opioid receptor antagonist is naloxone, naltrexone, nalmefene, or a combination of two or more thereof.
